(19) United States
(12) Patent Application Publication
(10) Pub. No.: US 2026/0098826 A1
(43) Pub. Date: Apr. 9, 2026

(54) AFFINITY CAPTURING AND DIRECTLY DETERMINING STRUCTURES OF PROTEINS AND OTHER MATERIALS ON SUPERPARAMAGNETIC BEADS BY CRYO-ELECTRON MICROSCOPY SINGLE-PARTICLE ANALYSIS

(71) Applicant: THE ROCKEFELLER UNIVERSITY, New York, NY (US)

(72) Inventors: Yasuhiro ARIMURA, New York, NY (US); Hide A. KONISHI, New York, NY (US); Hironori FUNABIKI, New York, NY (US)

(21) Appl. No.: 19/112,721

(22) PCT Filed: Sep. 19, 2023
(86) PCT No.: PCT/US2023/033105

§ 371 Date: Mar. 18, 2025

Related U.S. Application Data
(60) Provisional application No. 63/407,761, filed on Sep. 19, 2022.

(51) Int. Cl. G01N 23/2251; B82Y 15/00; G01N 33/53; G01N 33/543

(52) U.S. Cl. CPC G01N 23/2251; G01N 33/5308; G01N 33/5434; B82Y 15/00; G01N 2223/612

(57) ABSTRACT

Magnetic particles capture a biological target molecule for cryo-electron microscope imaging. The magnetic particles include paramagnetic beads. At least two spacer modules extend from a periphery of the paramagnetic beads comprising a first spacer module and a second spacer module. The first spacer module binds the nanoparamagnetic beads and the second spacer module is located outwardly of the first spacer module and binds the first spacer module. The first spacer module includes first spacer proteins and the second spacer module includes second spacer proteins. A capture module is linked to an outer location of the second spacer module. The capture module includes capture proteins that are adapted to capture target molecules. At least two spacer modules are arranged so that a combined length of the at least two spacer modules locates the capture module a distance that is spaced from the nanoparamagnetic beads. A method is also provided of using cryo-electron microcopy and the magnetic particles to image a biological target molecule.

### FIELD OF THE INVENTION

The present invention and disclosure relates to protein structural analysis, particularly cryo-electron microscopy (cryo-EM) single particle analysis for determining high-resolution three-dimensional (3D) structures of proteins and protein-bound complexes.

### SEQUENCE LISTING

A Sequence Listing conforming to the rules of WIPO Standard ST.26 is hereby incorporated by reference. The Sequence Listing has been filed as an electronic document via EFS-Web in ASCII format encoded as XML. The electronic document, created on Sep. 18, 2023, is entitled “1119-79-PCT_ST26.xml”, and is 79.853 bytes in size.

### BACKGROUND OF THE INVENTION

Protein structural analysis serves as a basis for understanding structures and interactions of at a highly accurate and focused level. Breakthroughs and new approaches have enabled structural analysis requiring vastly smaller amounts of material, while maintaining and even improving resolution. X-ray crystallography provides high resolution but requires a protein crystal and over 1 mg of protein. Nuclear magnetic resonance (NMR) utilizes can capture flexible structures and utilize protein solutions and requires over 10 mg of protein. Cryo-electron microscopy (Cryo-EM) provides high resolution utilizing protein solutions and requires smaller amounts of protein.

Cryo-EM is widely used to determine high-resolution three-dimensional (3D) structures of proteins and other protein-bound complexes such as protein-DNA complexes and protein-RNA complexes. Typically, highly concentrated aqueous samples (about 0.2-1 mg/ml proteins) are required for acquiring enough particle images on cryo-EM micrographs to reconstitute a 3D structure. The necessity of a high concentration sample makes it difficult to determine the structures of low-abundant proteins and protein complexes existing in a natural cellular context. One common approach to overcome this issue is to overexpress the recombinant proteins in cultured cells. However, this approach may miss the protein structural variation in a natural cellular context. In cells, proteins dynamically alter their structures and functions commonly (but not exclusively) via changing chemical modifications and/or ligands, as contexts, such as cell cycle, developmental, aging and pathological stages. If the recombinant proteins (e.g., those from human) are overexpressed in different species (e.g., in bacteria), the natural protein structural variations may not be reproduced. Even if the recombinant proteins are expressed in cells of the matching species, overexpressed proteins often fail to form functional complexes, or may form altered complexes due to the overexpression. Therefore, there is a need for methods and approaches to analyze the structures of low-abundant proteins and protein complexes in a natural cellular context.

Current cryo-EM methods and approaches are unable to readily determine structures of low abundant proteins (ug of proteins are required) and complexes comprising unknown components, particularly if ectopically expressed. Endogenous known proteins need to be part of the complex for structure determination with current methods. Further, current methods cannot address transient states wherein the protein changes structure with activation, inactivation, modification etc. The conventional cryo-EM sample preparation method has two significant issues that make the structural determination of low-abundance proteins/particles challenging. The first is the sample loss during conventional protein purification methods, such as isolation by chromatography columns, dialysis, and concentration by centrifugal filter units. Loss of the target particles by absorption to plastic and membrane surfaces is a common problem during purification of low-abundant proteins. The other is the loss during the cryo-EM grid freezing process. In conventional cryo-EM grid preparations, 2-4 μL of aqueous samples are applied onto a grid, and then most of the liquid that contains target particles is removed by a filter paper to form a thin ice layer on the grid, as the thick ice layer interferes the cryo-EM analysis. Since the target particles are dispersed in the liquid, this procedure removes a majority of the target particles in the liquid (Carragher et al., 2019). Although the single particle analysis to reconstitute the 3D cryo-EM structure only requires about 10⁵-10⁶ particles (around 10⁻¹⁸ mol [amol], about 0.1-1 pg of proteins), high concentration samples (2-4 μL of about 0.2-2 mg/ml, around 10⁻¹² mol [pmol], about 0.6-3 μg of proteins) are required due to this sample loss during the grid freezing process.

### SUMMARY OF THE INVENTION

This disclosure prevents sample loss during sample purification and the cryo-EM grid freezing process to determine the structures of low-abundance proteins and protein complexes existing in a natural cellular context, which is referred to herein as: Magnetic Isolation and Concentration (MagIC)-cryo-EM (FIG. 1A). By coating paramagnetic beads with a target-capturing module (e.g., nanobody, scFv, Fab) linked to optimized spacer modules, the method enables direct cryo-EM single particle analysis of the target particles that are enriched on the paramagnetic beads (FIG. 1B). By replacing the target-capturing module on the beads, the beads can capture a variety of target materials, such as proteins, DNA, and RNA. In addition, by concentrating the paramagnetic beads on cryo-EM grids using a magnetic force, the sample loss during the grid freezing process is significantly reduced (FIG. 1B). This disclosure enables cryo-EM single particle analysis with low-abundance proteins and protein complexes. This disclosure enables cryo-EM-mediated single particle analysis of proteins and protein complexes in a natural cellular context.

An aspect of the disclosure features magnetic particles that capture biological target molecules for structural determination and analysis. An aspect of the disclosure features magnetic particles that capture biological target molecules for cryo-electron microscope imaging. The magnetic particles include paramagnetic beads. At least two spacer modules extend from a periphery of the paramagnetic beads including a first spacer module and a second spacer module. The first spacer module binds the paramagnetic beads and the second spacer module is located outwardly of the first spacer module and binds the first spacer module. The first spacer module includes first spacer proteins and the second spacer module includes second spacer proteins. A capture module (target-capturing module) or one or more capture module (target-capturing module) is linked to an outer location of the at least two spacer modules. The capture module includes capture proteins that are adapted to capture biological target molecules by having affinity to the biological target molecules. The capture module includes or comprises one or more capture proteins that are adapted to capture biological target molecules by having affinity to the biological target molecules. The at least two spacer modules are arranged so that a combined length of the at least two spacer modules locates the capture module a distance that is spaced from the paramagnetic beads.

In an embodiment the magnetic particles are paramagnetic beads. In an embodiment the paramagnetic beads are not more than 100 nm in size, not more than 80 nm in size, not more than 60 nm in size, or not more than 50 nm in size.

In an embodiment the first spacer proteins and the second spacer proteins are distinct protein sequences. In an embodiment the first spacer proteins are type I spacer proteins and the second spacer proteins are type II spacer proteins. The first spacer proteins and the second spacer proteins can be linked end to end.

In an embodiment the first spacer proteins, together with the second spacer proteins, form rays extending outwardly from the paramagnetic beads.

In an embodiment the type I spacer proteins comprise one or more monomeric triple helical bundle (3HB) proteins. In an embodiment the type II spacer proteins comprise one or more single alpha helix (SAH) proteins.

In an embodiment streptavidin binds the paramagnetic beads. In one embodiment, the first spacer proteins comprise biotinylated monomeric triple helical bundle (3HB) proteins. The streptavidin on the beads binds biotin of the biotinylated monomeric triple helical bundle (3HB) proteins. In one embodiment, the second spacer proteins include biotinylated single alpha helix (SAH) proteins. A mono-SPY-tagged avidin tetramer is linked to an outer end portion of the biotinylated monomeric triple helical bundle (3HB) proteins and binds biotin of multiple of the biotinylated single alpha helix (SAH) proteins.

In an embodiment, the mono-SPY-tagged avidin tetramer is linked to an outer end portion of the biotinylated monomeric triple helical bundle (3HB) proteins by bonding between SPYcatcher003 and SPYtag003 moieties (Keeble et al . . . 2019; Zakeri et al., 2012). A SPYcatcher003 moiety is located on an end portion of the biotinylated monomeric triple helical bundle (3HB) proteins and is bonded to the SPYtag003 moiety located on the mono-SPY-tagged avidin tetramer.

In an embodiment, the at least two spacer modules include a first spacer module including type I spacer proteins and a second spacer module including the type I spacer proteins. The first spacer module and the second spacer module can be linked end to end.

In an embodiment the first spacer module together with the second spacer module can form rays extending outwardly from the paramagnetic beads.

In an embodiment the type I spacer proteins include one or more single alpha helix (SAH) proteins.

In an embodiment streptavidin binds the paramagnetic beads. The first spacer proteins and the second spacer proteins include onenor more single alpha helix proteins. The streptavidin on the beads binds biotin of the biotinylated single alpha helix proteins of the first spacer module. A mono-SPY-tagged avidin tetramer is linked to an outer end portion of the biotinylated single alpha helix proteins of the first spacer module and binds biotin of multiple of the biotinylated single alpha helix proteins of the second spacer module.

In an embodiment the mono-SPY-tagged avidin tetramer is linked to an outer end portion of the single alpha helix proteins of the first spacer module by bonding between SPYcatcher003 and SPYtag003 moieties. A SPYcatcher003 moiety is located on an end portion of the biotinylated single alpha helix proteins of the first spacer module and is bonded to the SPYtag003 moiety located on the mono-SPY-tagged avidin tetramer.

In an embodiment the capture module is adapted to capture the at least one biological target molecule selected from at least one of a protein. RNA or DNA, and portions or fragments thereof, and combinations thereof. The capture module can use any or all suitable protein affinity purification systems (Brizzard, 2008; Pina et al., 2014) and DNA/RNA sequence specific affinity purification systems (Fujita and Fujii, 2019; Karkare and Bhatnagar, 2006; Wages et al., 1997), all of these references being incorporated herein by reference in their entiretics.

In an embodiment the paramagnetic beads are superparamagnetic.

In an embodiment the capture module includes an antibody, an antibody fragment, or an antigen binding portion of an antibody or antibody fragment. In an embodiment the capture module includes an antibody, antibody portion or antibody fragment.

In an embodiment the capture module is a protein affinity purification system including at least one of a Protein A. Strep-Tactin, calmodulin binding protein, single stranded DNA, double stranded DNA, single stranded RNA, double stranded RNA. SPYcatcher, streptavidin, streptavidin that binds NTA-biotin, streptavidin that binds biotinylated glutathione, streptavidin that binds Tris-NTA-biotin, streptavidin that binds Halotag biotin ligand, streptavidin that binds chitosan-biotin, or streptavidin that binds SNAP-biotin.

In an embodiment the capture module is a DNA/RNA sequence-based affinity system including at least one of a nuclease-dead CRISPR-associated protein 9 (dCas9) attached with single guide RNA (sgRNA), zinc-finger proteins (ZFPs), transcription activator-like (TAL) proteins, PNA, LNA, single stranded DNA, single stranded RNA, or morpholino.

In an embodiment the capture module includes at least one of a nanobody, scFv or Fab.

In an embodiment the capture module includes a nanobody with affinity for a protein or a fluorescent protein tag of the protein.

In an embodiment the at least one nanobody, scFv or Fab has affinity for a histone or a tag of the histone.

In an embodiment the at least one nanobody, scFv or Fab has affinity for a protein or a tag of the protein, and the protein binds to DNA.

In an embodiment the at least one nanobody, scFv or Fab has affinity for a protein or a tag of the protein, and the protein is associated with at least one other protein that binds to DNA.

In an embodiment the distance that is spaced from the paramagnetic beads is at least about 30 nm, at least about 40 nm, at least about 50 nm, at least about 60 nm, at least about 70 nm or at least about 80 nm.

A second aspect of the disclosure is a method of using cryo-electron microcopy to image a biological target molecule including the following steps. Any of the magnetic particles described in the present disclosure are provided. The magnetic particles are mixed in a liquid with target molecules to capture the biological target molecules with the capture protcins. A liquid including the magnetic particles with captured biological target molecules is applied onto an electron microscope grid. The biological target molecules are concentrated in the liquid on the electron microscope grid by applying a magnetic field to the magnetic particles with captured target molecules. A portion of the liquid is removed from the electron microscope grid. Cryogenic conditions are applied so as to vitrify the magnetic particles and captured biological target molecules on the electron microscope grid. Electron microscope imaging of the biological target molecules is conducted.

In an embodiment the method includes applying a sheet of material onto the electron microscope grid, and applying the liquid including the magnetic particles with captured biological target molecules onto the sheet of material on the electron microscope grid. The rest of the method steps of the third aspect are then carried out (concentrating the liquid on the electron microscope grid; removing a portion of the liquid; applying cryogenic conditions; and conducting electron microscope imaging of the biological target molecules).

In an embodiment of the method the sheet of material includes graphene.

In an embodiment of the method the electron microscope imaging enables single particle analysis of the biological target molecules.

In an embodiment of the method the capture module is a protein affinity purification system including at least one of Protein A. Strep-Tactin, calmodulin binding protein, single stranded DNA, double stranded DNA, single stranded RNA, double stranded RNA. SPYcatcher, streptavidin, streptavidin that binds NTA-biotin, streptavidin that binds biotinylated glutathione, streptavidin that binds Tris-NTA-biotin, streptavidin that binds Halotag biotin ligand, streptavidin that binds chitosan-biotin, or streptavidin that binds SNAP-biotin.

In an embodiment of the method the capture module is a DNA/RNA sequence-based affinity system including at least one of nuclease-dead CRISPR-associated protein 9 (dCas9) attached with single guide RNA (sgRNA), zinc-finger proteins (ZFPs), transcription activator-like (TAL) proteins, PNA, LNA, single stranded DNA, single stranded RNA, or morpholino.

In an embodiment of the method the capture module includes an antibody, an antibody fragment, or an antigen binding portion of an antibody or antibody fragment. In an embodiment of the method the capture module includes an antibody, antibody portion or antibody fragment.

In an embodiment of the method the capture module includes at least one of a nanobody, scFv or Fab.

In an embodiment of the method the capture module includes a nanobody with affinity for a protein or a fluorescent protein tag of the protein that forms a part of the biological target molecules.

In an embodiment of the method the at least one of a nanobody, scFv or Fab has affinity for a tag of a linker histone, the linker histone being bound to a nucleosome. The linker histone is captured and electron microscope imaging is conducted on at least one of the nucleosome and the captured linker histone as the biological target molecules.

In an embodiment the method includes capturing in vitro reconstituted recombinant histone-bound nucleosomes. Electron microscope imaging is conducted on the recombinant histone-bound, captured nucleosomes.

In an embodiment the method includes capturing recombinant histone-bound nucleosomes, which are isolated from chromosomes in a cellular environment. Electron microscope imaging is conducted on the recombinant histone-bound captured nucleosomes.

In an embodiment of the method the tag is at least one of a GFP tag, Myc tag, HA tag, V5-tag. CD tag. or FLAG tag, and combinations thereof.

In an embodiment of the method the tag is a peptide and protein affinity tag including at least one of SPYtag, CBP-tag, GST-tag, poly His-tag or SNAP-tag, CDB tag, Halo tag, Avitag, S-tag, or Strep-tag, and combinations thereof.

In an embodiment of the method the nanobody has affinity for GFP as the tag.

In an embodiment of the method the at least one nanobody, scFv or Fab has affinity for a protein or a tag of a protein, the protein binding DNA. The protein is captured and electron microscope imaging of the protein is conducted.

In an embodiment of the method the at least one nanobody, scFv or Fab has affinity for a protein or a tag of the protein, the protein being associated with at least one other protein that binds to DNA. The protein is captured and electron microscope imaging of the protein is conducted.

An embodiment of the method includes capturing a biological target molecule selected from at least one of a protein. RNA or DNA, and portions or fragments thereof, and combinations thereof, and conducting electron microscope imaging thereof.

In an embodiment of the method the target molecules are isolated from a low purity sample. In an embodiment of the method the target molecules are isolated at low concentrations. In an embodiment of the method the target molecules are isolated from a low purity sample and at low concentrations. In an embodiment target molecules are captured and structure can be determined wherein the target complex is at nanomolar (nM) concentrations. In embodiments target complex is captured at concentrations of less than 20 nM, less than 10 nM, less than 5 nM, less than 2 nM, at 1-20 nM, at 1-10 nM, at 1-5 nM at 1-2 nM. In embodiments target complex is captured and structure can be determined at concentrations of less than 20 nM, less than 10 nM, less than 5 nM, less than 2 nM, at 1-20 nM, at 1-10 nM, at 1-5 nM at 1-2 nM.

In embodiments target complex is captured at concentrations of less than 20 ng/ml, less than 10 ng/ml, less than 5 ng/ml, less than 3 ng/ml, less than 2 ng/ml, less than 1 ng/ml, about 1-1.5 ng/ml, about 1 ng/ml, less than 1 ng/ml, about 0.5 ng/ml. In embodiments target complex is captured and structure can be determined at concentrations of less than 20 ng/ml, less than 10 ng/ml, less than 5 ng/ml, less than 3 ng/ml, less than 2 ng/ml, less than 1 ng/ml, about 1-1.5 ng/ml, about 1 ng/ml, less than 1 ng/ml, about 0.5 ng/ml.

In an embodiment of the method the biological target molecules are captured while present in a solution in a concentration of not more than 1 nM, not more than 1.6 nM, not more than 5 nM, not more than 10 nM, not more than 20 nM, not more than 30 nM, not more than 34 nM, not more than 40 nM, not more than 60 nM, not more than 80 nM, or not more than 100 nM. For example, protein and DNA as the biological target molecules can be recombinant H1.8-GFP bound nucleosome in a solution in a concentration of not more than 5 nM or not more than 10 nM, which are captured by the magnetic particles of this disclosure.

A third aspect of the disclosure is a method of making magnetic particles for capturing biological target molecules for cryo-electron microscope imaging, including the following steps:
a) providing paramagnetic beads conjugated with streptavidin;
b) adding biotin-3HB-SPYcatcher003 proteins or biotin-SAH-SPYcatcher003 proteins to the paramagnetic beads conjugated with streptavidin, wherein the biotin of the biotin-3HB-SPYcatcher003 proteins binds the streptavidin, or the biotin of the biotin-SAH-SPYcatcher003 proteins binds the streptavidin, attaching a first spacer module;
c) adding mono-SPY-tagged avidin tetramer proteins to the first module, wherein the SPYtag003 moiety of the mono-SPY-tagged avidin tetramer proteins bonds the SPYcatcher003 moiety of the biotin-3HB-SPYcatcher003 proteins, or wherein the SPYtag003 moiety of the mono-SPY-tagged avidin tetramer proteins bonds the SPYcatcher003 moiety of the biotin-SAH-SPYcatcher003 proteins; and
d) adding second spacer module biotinylated proteins including SAH and SPYcatcher003 moieties to the first spacer module and the bonded mono-SPY-tagged avidin tetramer proteins, to attach the second spacer module, wherein the biotin of the second spacer module biotinylated proteins binds to the avidin of the mono-SPY-tagged avidin tetramer proteins.

In an embodiment of the method at least one buffer is present in any one or more of steps a)-d).

An embodiment of the method includes e) providing a capture module including a SPYtag003 moiety and at least one capturing polypeptide that is adapted to have affinity to biological target molecules, adding the capture module to the second spacer module, wherein the SPYtag003 moiety of the capture module bonds to the SPYcatcher003 moiety of the second spacer module biotinylated proteins.

In an embodiment of the method the capture module includes an antibody, antibody portion or antibody fragment.

In an embodiment of the method the capture module is a protein affinity purification system including at least one of Protein A. Strep-Tactin, calmodulin binding protein, single stranded DNA, double stranded DNA, single stranded RNA, double stranded RNA. SPYcatcher, streptavidin, streptavidin that binds NTA-biotin, streptavidin that binds biotinylated glutathione, streptavidin that binds Tris-NTA-biotin, streptavidin that binds Halotag biotin ligand, streptavidin that binds chitosan-biotin, or streptavidin that binds SNAP-biotin.

In an embodiment of the method the capture module is a DNA/RNA sequence-based affinity system including at least one of a nuclease-dead CRISPR-associated protein 9 (dCas9) attached with single guide RNA (sgRNA), zinc-finger proteins (ZFPs), transcription activator-like (TAL) proteins, PNA. LNA, single stranded DNA, single stranded RNA, or morpholino.

In an embodiment of the method the capture module includes at least one of a nanobody, scFv or Fab.

In an embodiment of the method the capture module has affinity for a protein or a tag of the protein that forms a part of the biological target molecules.

In an embodiment of the method the at least one of a nanobody, scFv or Fab has affinity for a linker histone including a tag, and the linker histone binds a nucleosome.

In an embodiment of the method the capture module is adapted to capture in vitro reconstituted recombinant histone-bound nucleosomes as the biological target molecules.

In an embodiment of the method the capture module is adapted to capture recombinant histone-bound nucleosomes as the biological target molecules, which have been isolated from chromosomes in a cellular environment.

In an embodiment of the method the tag is at least one of a GFP tag, Myc tag, HA tag, V5-tag, CD tag, or FLAG tag, and combinations thereof.

In an embodiment of the method the tag is a peptide and protein affinity tag including at least one of SPYtag, CBP-tag, GST-tag, poly His-tag or SNAP-tag, CDB tag, Halo tag, Avitag, S-tag, or Strep-tag and combinations thereof.

In an embodiment of the method the at least one nanobody, scFv or Fab has affinity for a protein or a tag thereof, and the protein binds DNA.

In an embodiment of the method the at least one nanobody, scFv or Fab has affinity for a protein or a tag thereof.

In an embodiment of the method the at least one nanobody, scFv or Fab has affinity for a protein or a tag of the protein, the protein being associated with at least one other protein that bind to DNA.

In an embodiment of the method the capture module is adapted to capture the biological target molecules selected from at least one of a protein, RNA or DNA, and portions or fragments thereof, and combinations thereof.

A fourth aspect of the disclosure is a combination product including magnetic particles that capture a biological target molecule for cryo-electron microscope imaging including:
a) paramagnetic beads conjugated with streptavidin;
b) optional biotin-3HB-SPYcatcher003 proteins;
c) biotinylated proteins including SAH and SPYcatcher003 moieties;
d) mono-SPY-tagged avidin tetramer proteins; and
e) a capture module including a SPYtag003 moiety and at least one capturing polypeptide that is adapted to have affinity to a biological target molecule.

In an embodiment of the combination product at least two of components a), b), c), d) and c) can be provided in separate packaging.

In an embodiment, the combination product includes at least one buffer containing one or more of components a)-e).

In an embodiment of the combination product the capture module is a protein affinity purification system including at least one of Protein A. Strep-Tactin, calmodulin binding protein, single stranded DNA, double stranded DNA, single stranded RNA, double stranded RNA, SPYcatcher, streptavidin, streptavidin that binds NTA-biotin, streptavidin that binds biotinylated glutathione, streptavidin that binds Tris-NTA-biotin, streptavidin that binds Halotag biotin ligand, streptavidin that binds chitosan-biotin, or streptavidin that binds SNAP-biotin.

In an embodiment of the combination product the capture module is a DNA/RNA sequence-based affinity system including at least one of a nuclease-dead CRISPR-associated protein 9 (dCas9) attached with single guide RNA (sgRNA), zinc-finger proteins (ZFPs), transcription activator-like (TAL) proteins. PNA, LNA, single stranded DNA, single stranded RNA, or morpholino.

In an embodiment of the combination product the capture module includes an antibody, antibody portion or antibody fragment.

In an embodiment of the combination product the capture module includes at least one of a nanobody, scFv or Fab.

In an embodiment of the combination product the capture module has affinity to a protein or a tag of the protein that forms a part of the biological target molecules.

In an embodiment of the combination product the at least one of a nanobody, scFv or Fab has affinity for a linker histone including a tag, and the linker histone binds a nucleosome.

In an embodiment of the combination product the capture module is adapted to capture in vitro reconstituted recombinant histone-bound nucleosomes as the biological target molecules.

In an embodiment of the combination product the capture module is adapted to capture recombinant histone-bound nucleosomes as the biological target molecules, which have been isolated from chromosomes in a cellular environment.

In an embodiment of the combination product the tag includes at least one of a GFP tag, Myc tag. HA tag, V5-tag. CD tag, or FLAG tag, and combinations thereof.

In an embodiment of the combination product the tag is a peptide and protein affinity tag including at least one of SPYtag, CBP-tag, GST-tag, poly His-tag or SNAP-tag, CDB tag, Halo tag, Avitag. S-tag, or Strep-tag and combinations thereof.

In an embodiment of the combination product the at least one nanobody, scFv or Fab has affinity for a protein or a tag thereof, and the protein binds DNA.

In an embodiment of the combination product the at least one nanobody, scFv or Fab has affinity for a protein or a tag of the protein, the protein being associated with at least one other protein that bind to DNA.

In an embodiment of the combination product the at least one nanobody, scFv or Fab has affinity for a protein or a tag thereof.

In an embodiment of the combination product the capture module has affinity for the biological target molecules selected from at least one of a protein, RNA or DNA, and portions or fragments thereof, and combinations thereof.

Other objects and advantages will become apparent to those skilled in the art from a review of the ensuing detailed description, which proceeds with reference to the following illustrative drawings, and the attendant claims.

### BRIEF DESCRIPTION OF THE DRAWINGS

FIG. 1A provides a diagram showing the principal scheme of this disclosure. Target proteins (or protein complexes) are captured to paramagnetic beads through target-capturing modules (e.g., nanobody, scFv, and Fab) with spacer modules conjugated on the beads. The beads are then concentrated on the cryo-EM grid using a magnetic force. FIG. 1B shows diagrams depicting the two distinct features, “magnetic target isolation” and “magnetic concentration on EM grid”, of MagIC-cryo-EM compared to the conventional cryo-EM method. By isolating target proteins with paramagnetic beads, sample loss during purification steps is reduced. In addition, by concentrating the paramagnetic beads on cryo-EM grids using a magnetic force, the sample loss during the grid freezing process is reduced.

FIG. 2 is a graphical representation showing the sample preparation schemes used in Examples 2-7. The left panel shows the sample used in Examples 2-4, biotin-labeled poly-nucleosome (biotin attached to an end of the linear 3.8 kb DNA on which up to 19 nucleosome complexes are assembled) reconstituted in vitro. The middle panel shows the sample used in Examples 5-6, H1.8-GFP bound mono-nucleosome reconstituted in vitro. The right panel shows the sample used in Example 7, nucleosomes isolated from the metaphase chromosomes formed in the Xenopus egg extract containing H1.8-GFP.

FIG. 3 is a graphical representation showing the nano-magnetic beads used in Examples 2-7. The left panel shows the nano-magnetic beads used in Examples 2-4, 50 nm Absolute Mag streptavidin magnetic beads purchased from CD Bioparticles (WHM-X047). The middle panel shows the nano-magnetic beads used in Example 5, 3HB-30 nm-SAH GFP nanobody beads. To make the captured protein complexes away from magnetic beads, two types of spacer modules, biotin-monomeric triple helical bundle-SPYcatcher003 (biotin-3HB-SPYcatcher003) and biotin-30 nm single alpha helix-SPYcatcher003 (biotin-30 nm-SAH-SPYcatcher003), are attached to the Absolute Mag streptavidin magnetic beads (WHM-X047). Branching the biotin-30 nm-SAH-SPYcatcher003 using mono-SPYtag-avidin-tetramer quadruples the number of available target-capturing modules. At the distal end of the spacer proteins, SPYtag-tandem GFP nanobodies are conjugated to capture GFP-tagged protein complexes. The right panel shows the nano-magnetic beads used in Examples 6-7, 30 nm-SAH×2 GFP nanobody beads. To increase the spacer length and flexibility, two of the biotin-30 nm-SAH-SPYcatcher003 are attached in tandem to the Absolute Mag streptavidin magnetic beads (WHM-X047).

FIG. 4 shows the cryo-EM micrographs of the nano-magnetic beads coated with poly-nucleosome (biotin attached to an end of the linear 3.8 kb DNA on which up to 19 nucleosome complexes are assembled), as explained in example 2. The left panel is the low magnification (×2.600) cryo-EM micrograph of the nano-magnetic beads capturing the biotin-poly-nucleosome. Although nucleosomes (target particles) cannot be seen at this magnification, magnetic beads can be easily found as black dots. Therefore, by using magnetic beads, the grid well where target particles are deposited can be easily identified by taking the low magnification micrograph. The middle panel is the high magnification (x28,000) micrograph used for 3D structure reconstruction. The right panel is the annotation of the micrograph shown in the middle. The dark granular clusters marked with asterisks are the magnetic beads with about 50 nm diameter. White halos with 20-30 nm width, which interferes with sample particles at the vicinity of the beads, are observed around each bead. The circles indicate the nucleosome particles selected manually. Due to the noise, nucleosomes more than 30 nm away from beads could be used for the structural analysis.

FIG. 5 shows the cryo-EM structure of the nucleosome reconstituted from the 657 micrographs of nano-magnetic beads capturing biotin-poly-nucleosome shown in FIG. 4.

FIG. 6 shows the effect of the magnetic concentration of the beads. The upper diagrams depict the principle of magnetic concentration. To prevent sample loss during the grid freezing process, the magnetic beads are concentrated on monolayer graphene-coated cryo-EM grids using a magnet placed in a humidity chamber. The middle and right panel is the low magnification (x2600) cryo-EM micrograph without and with magnetic concentration, respectively. The magnetic beads are efficiently concentrated on cryo-EM grids by the magnetic concentration.

FIG. 7 shows the effect of the graphene during magnetic concentration. Left panels are the low magnification (x2.600) cryo-EM micrograph and representation of the magnetic concentrated nano-magnetic beads capturing biotin-poly-nucleosome on the quantifoil grid without monolayer graphene. Without monolayer graphene, most of the nucleosomes are broken, likely due to the exposure to the air-water interface. Right panels are the low magnification (x2,600) cryo-EM micrograph and representation of the magnetic concentrated nano-magnetic beads capturing biotin-poly-nucleosome quantifoil grid with monolayer graphene. By using the monolayer graphene-coated grid, nucleosomes are still intact after freezing.

FIG. 8 provides a diagram (left) and sodium dodecyl sulfate-polyacrylamide gel electrophoresis (SDS-PAGE) analysis (right) showing that 3HB-30 nm-SAH GFP nanobody beads specifically isolate GFP-bound complex by chromatin immunoprecipitation (ChIP). Nucleosomes and H1.8-GFP bound nucleosomes are reconstituted in vitro and mixed with the 3HB-30 nm-SAH beads with or without His₆-SPYtag-tandem GFP nanobody. The nano-magnetic beads are collected and washed with centrifugation and analyzed with SDS-PAGE and gel code blue staining. The H1.8-GFP bound nucleosomes are specifically isolated by nano-magnetic beads only when the His₆-SPYtag-tandem GFP nanobody exist. Here. 100 ul of a 10 mg/ul sample or 10 pmol of H1.8-GFP bound nucleosome is applied. About 50% of the beads were used for SDS-PAGE and 10% of the beads were used for cryo-EM. In this study, a single magnetic bead binds around 100-2500 H1.8GFP.

FIG. 9 shows the cryo-EM micrographs of the 3HB-30 nm-SAH GFP nanobody beads capturing H1.8-GFP bound nucleosome (shown in FIG. 8). The left panel is the low magnification (x2.600) cryo-EM micrograph. Dense about 50 nm granules on the micrograph are magnetic beads. Each magnetic bead is well separated due to the spacer protein. The middle panel is the high magnification (x28,000) micrograph that is used for 3D structure reconstruction. The right panel is the annotation of the micrograph shown in the middle. The magnetic beads are seen as strong blob-shaped signals. The spacer proteins form a 40-50 nm radius layer around each magnetic bead, covering the white halo-shaped noise. The circles indicate the H1.8-GFP bound nucleosome particles captured by 3HB-30 nm-SAH GFP nanobody beads. The particles on the micrograph are picked by Topaz software.

FIG. 10 shows the cryo-EM structure of the H1.8-GFP bound nucleosome reconstituted from the 1,131 micrographs of 3HB-30 nm-SAH GFP nanobody beads capturing H1.8-GFP bound nucleosome shown in FIG. 9. The left panel is the cryo-EM map, and the right panel is the superimposition of the cryo-EM map and the atomic-model of H1.8 bound nucleosome (PDB ID: 7KBF). The black ribbon model indicates H1.8.

FIG. 11 shows the cryo-EM micrographs of the 30 nm-SAH×2 GFP nanobody beads capturing H1.8-GFP bound nucleosome. The left panel is the low magnification (x2,600) cryo-EM micrograph. Black dots on the micrograph are magnetic beads. The middle panel is the high magnification (x28,000) micrograph used for 3D structure reconstruction. The right panel is the annotation of the micrograph shown in the middle. Strong blob-shaped signals represent a magnetic bead. The spacer proteins form a 50-70 nm-layer around the magnetic bead, covering the white halo-shaped noise. The circles indicate the H1.8-GFP bound nucleosome particles captured by 30 nm-SAH×2 GFP nanobody beads. The particles on the micrograph are picked by Topaz software.

FIG. 12 shows the cryo-EM structure of the H1.8-GFP bound nucleosome reconstituted from 510 micrographs of 3HB-30 nm-SAH GFP nanobody beads capturing the H1.8-GFP bound nucleosome shown in FIG. 11. The left panel is the cryo-EM map, and the right panel is the superimposition of the cryo-EM map and the atomic-model of H1.8 bound nucleosome (PDB ID: 7KBF). The black ribbon model indicates H1.8.

FIG. 13A shows the situation isolating low-abundant proteins, 100 μL of 0.28, 1.4, 2.8 ng DNA and protein/μL (1, 5, and 10 nM) of H1.8-GFP bound nucleosome (looking at the figures left to right) used in MagIC-cryo-EM. 3HB-60 nm-SAH GFP nanobody beads capturing H1.8-GFP bound nucleosomes are loaded onto a cryo-EM grid coated with a monolayer graphene, concentrated by a magnetic force, and plunge frozen. The circles indicate the H1.8-GFP bound nucleosome particles captured by 3HB-60 nm-SAH GFP nanobody beads. The particles on the micrograph are manually picked. When using 100 u L, of 0.28 ng/u I. H1.8-GFP bound nucleosomes, more than 20 nucleosomes arc observed around a single bead on a cryo-EM micrograph (FIG. 13A bottom left). The numbers of the nucleosomes that can be used for structural analysis are significantly increased when using 100 μL of 1.4 ng/μL H1.8-GFP bound nucleosomes (FIG. 13A bottom middle). The further increase of the concentration to 100 μL of 2.8 ng/μL does not improve the numbers of the usable nucleosome on micrograph (FIG. 13A bottom right).

FIG. 13B shows the cryo-EM micrographs of the 3HB-60 nm-SAH GFP nanobody beads capturing H1.8-GFP bound nucleosome. The left panel is the low magnification (x2,600) cryo-EM micrograph. Black dots on the micrograph are magnetic beads. The middle panel is the high magnification (x28,000) micrograph used for 3D structure reconstruction. The right panel is the annotation of the micrograph shown in the middle. Strong blob-shaped signals represent a magnetic bead. The spacer proteins form a 50-70 nm-layer around the magnetic bead, covering the white halo-shaped noise. The circles indicate the H1.8-GFP bound nucleosome particles captured by 3HB-60 nm-SAH GFP nanobody beads. The particles on the micrograph are picked by Topaz software.

FIG. 14 shows the cryo-EM structure of the H1.8-GFP bound nucleosome reconstituted from 510 micrographs of 3HB-60 nm-SAH GFP nanobody beads capturing the H1.8-GFP bound nucleosome shown in FIG. 13. The left panel is the cryo-EM map, and the right panel is the superimposition of the cryo-EM map and the atomic-model of H1.8 bound nucleosome (PDB ID: 7KBF). The black ribbon model indicates H1.8.

FIG. 15 shows the SDS-PAGE gel of the 3HB-30 nm-SAH GFP nanobody beads capturing H1.8-GFP bound nucleosome isolated from metaphase chromosomes formed in Xenopus egg extract stained with silver staining. The H1.8-GFP and histones bands are observed on the 3HB-30 nm-SAH GFP nanobody beads, suggesting that H1.8-GFP bound nucleosomes are specifically captured by 3HB-30 nm-SAH GFP nanobody beads as expected.

FIG. 16 shows the cryo-EM micrographs of the 3HB-30 nm-SAH GFP nanobody beads capturing H1.8-GFP bound nucleosome isolated from metaphase chromosomes formed in Xenopus egg extract. The top panel is the low magnification (x2,600) cryo-EM micrograph. Black dots on the micrograph are magnetic beads. The middle panels are the high magnification (x28,000) micrograph of the 3HB-30 nm-SAH GFP nanobody beads capturing H1.8-GFP bound nucleosome isolated from metaphase chromosomes formed in Xenopus egg extract. The bottom panels are the high magnification (x28,000) micrograph of sucrose gradient fractions of the mctaphase chromosomes isolated in Xenopus egg extract (Arimura et al., 2021). The circles indicate the H1.8-GFP bound nucleosome particles captured by 3HB-30 nm-SAH GFP nanobody beads. The particles on the micrograph are picked by Topaz software. Although many non-nucleosome proteins are seen on the sucrose gradient fraction of metaphase chromosomes without MagIC-cryo-EM, nucleosome-like particles are specifically enriched around the beads on MagIC-cryo-EM micrograph and no contaminated protein are seen outside the beads.

FIG. 17 shows the cryo-EM structure of the H1.8-GFP bound nucleosome isolated from metaphase chromosomes formed in Xenopus egg extract. The structure is reconstituted from 905 micrographs shown in FIG. 16. The left panel is the cryo-EM map, and the right panel is the superimposition of the cryo-EM map and the atomic-model of nucleosome (PDB ID: 7KBE).

FIG. 18 provides a diagram (left) and electrophoresis analysis (right) showing that nanobody beads specifically isolate H1.8 GFP-bound nucleosomes from a low purity sample in the presence of an excess amount of nucleosomes. Nucleosomes and H1.8-GFP bound nucleosomes are reconstituted in vitro and mixed. The nano-magnetic beads are collected and washed with centrifugation and analyzed. The H1.8-GFP bound nucleosomes are isolated by nano-magnetic beads.

FIG. 19 provides cryo-EM micrographs of the 3HB-60 nm-SAH GFP nanobody beads capturing H1.8-GFP bound nucleosome isolated from low purity samples.

FIG. 20 provides the H1.8-GFP bound nucleosome structure at 3.8 Å.

FIG. 21 provides a diagram (left) and native PAGE electrophoresis analysis (right) showing that nanobody beads specifically isolate H1.8 GFP-bound nucleosomes from a crude cell fraction, wherein they are isolated from interphase and metaphase chromosomes in Xenopus egg extract. The nano-magnetic beads are collected and washed with centrifugation and analyzed. The H1.8-GFP bound nucleosomes are isolated by nano-magnetic beads from interphase and metaphase chromosomes in the egg extracts.

FIG. 22 provides cryo-EM micrographs of the 3HB-60 nm-SAH GFP nanobody beads capturing H1.8-GFP bound nucleosome isolated from Xenopus egg extract chromatin.

FIG. 23 provides the H1.8-GFP bound nucleosome structure isolated from interphase Xenopus egg extract at 4.0 Å and isolated from metaphase Xenopus egg extract at 3.9 Å.

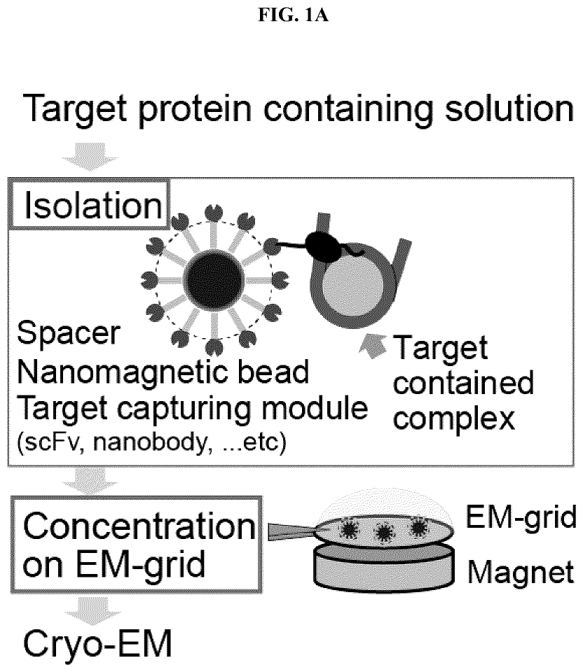

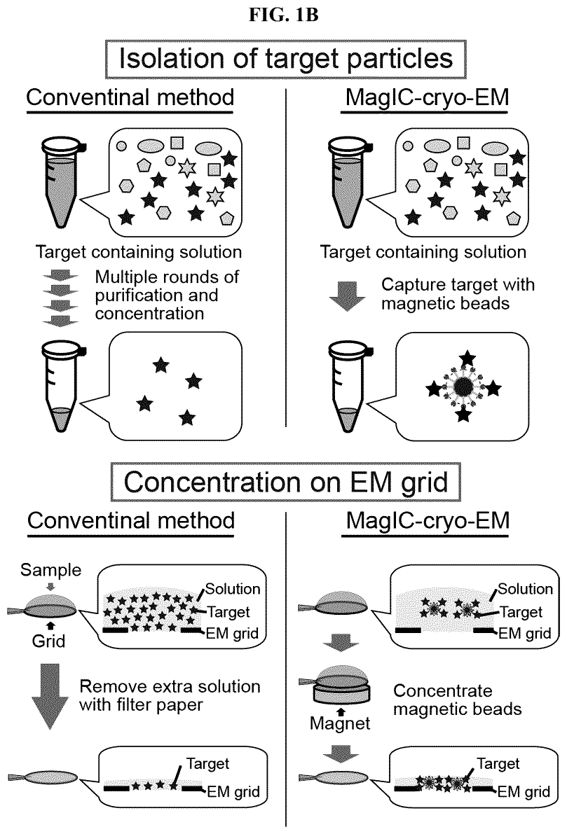

### DETAILED DESCRIPTION

In accordance with the present disclosure there may be employed conventional molecular biology, microbiology, and recombinant DNA techniques within the skill of the art. Such techniques are explained fully in the literature. See, e.g., Sambrook et al, “Molecular Cloning: A Laboratory Manual” (1989); “Current Protocols in Molecular Biology” Volumes I-III [Ausubel, R. M., ed. (1994)]; “Cell Biology: A Laboratory Handbook” Volumes I-III [J. E. Celis, ed. (1994))]; “Current Protocols in Immunology” Volumes I-III [Coligan. J. E., ed. (1994)]; “Oligonucleotide Synthesis” (M. J. Gait ed. 1984); “Nucleic Acid Hybridization” [B. D. Hames & S. J. Higgins eds. (1985)]; “Transcription And Translation” [B. D. Hames & S. J. Higgins, eds. (1984)]; “Animal Cell Culture” [R. I. Freshney, ed. (1986)]; “Immobilized Cells And Enzymes” [IRL Press, (1986)]; B. Perbal, “A Practical Guide To Molecular Cloning” (1984).

Therefore, if appearing herein, the following terms shall have the definitions set out below.

### A. Terminology

The term “antibody” describes an immunoglobulin whether natural or partly or wholly synthetically produced. The term also covers any polypeptide or protein having a binding domain which is, or is homologous to, an antibody binding domain. CDR grafted antibodies are also contemplated by this term. An “antibody” is any immunoglobulin, including antibodies and fragments thereof, that binds a specific epitope. The term encompasses polyclonal, monoclonal, and chimeric antibodies. The term “antibody (ies)” includes a wild type immunoglobulin (lg) molecule, generally comprising four full length polypeptide chains, two heavy (H) chains and two light (L) chains, or an equivalent Ig homologue thereof (e.g., a camelid nanobody, which comprises only a heavy chain); including full length functional mutants, variants, or derivatives thereof, which retain the essential epitope binding features of an Ig molecule, and including dual specific, bispecific, multispecific, and dual variable domain antibodies; Immunoglobulin molecules can be of any class (e.g., IgG, IgE. IgM. IgD. IgA, and IgY), or subclass (e.g., IgG1, IgG2. IgG3, IgG4, IgA1, and IgA2). Also included within the meaning of the term “antibody” are any “antibody fragment”.

An “antibody fragment” means a molecule comprising at least one polypeptide chain that is not full length, including (i) a Fab fragment, which is a monovalent fragment consisting of the variable light (VL), variable heavy (VH), constant light (CL) and constant heavy 1 (CH1) domains; (ii) a F(ab′) 2 fragment, which is a bivalent fragment comprising two Fab fragments linked by a disulfide bridge at the hinge region; (iii) a heavy chain portion of an Fab (Fd) fragment, which consists of the VH and CH1 domains; (iv) a variable fragment (Fv), which consists of the VL and VH domains of a single arm of an antibody. (v) a domain antibody (dAb) fragment, which comprises a single variable domain (Ward. E. S. et al., Nature 341, 544-546 (1989)); (vi) a camelid antibody; (vii) an isolated complementarity determining region (CDR); (viii) a Single Chain Fv Fragment wherein a VH domain and a VL domain are linked by a peptide linker which allows the two domains to associate to form an antigen binding site (Bird et al, Science, 242, 423-426, 1988; Huston et al. PNAS USA. 85, 5879-5883, 1988); (ix) a diabody, which is a bivalent, bispecific antibody in which VH and VL domains are expressed on a single polypeptide chain, but using a linker that is too short to allow for pairing between the two domains on the same chain, thereby forcing the domains to pair with the complementarity domains of another chain and creating two antigen binding sites (WO94/13804; P. Holliger et al Proc. Natl. Acad. Sci. USA 90 6444-6448, (1993)); and (x) a linear antibody, which comprises a pair of tandem Fv segments (VH-CH1-VH-CH1) which, together with complementarity light chain polypeptides, form a pair of antigen binding regions; (xi) multivalent antibody fragments (scFv dimers, trimers and/or tetramers (Power and Hudson, J Immunol. Methods 242:193-204 9 (2000)); (xii) a minibody, which is a bivalent molecule comprised of scFv fused to constant immunoglobulin domains, CH3 or CH4, wherein the constant CH3 or CH4 domains serve as dimerization domains (Olafsen T et al (2004) Prot Eng Des Sel 17 (4): 315-323; Hollinger P and Hudson PJ (2005) Nature Biotech 23 (9): 1126-1136); and (xiii) other non-full length portions of heavy and/or light chains, or mutants, variants, or derivatives thereof, alone or in any combination.

As antibodies can be modified in a number of ways, the term “antibody” should be construed as covering any specific binding member or substance having a binding domain with the required specificity. Thus, this term covers antibody fragments, derivatives, functional equivalents and homologues of antibodies, including any polypeptide comprising an immunoglobulin binding domain, whether natural or wholly or partially synthetic. Chimeric molecules comprising an immunoglobulin binding domain, or equivalent, fused to another polypeptide are therefore included.

The term “binding” refers to a direct association between molecules and/or atoms, due to, for example, covalent, electrostatic, hydrophobic, and ionic and/or hydrogen-bond interactions, including interactions such as salt bridges and water bridges. The term “bonding” as used herein generally refers to covalent bonding.

The term affinity” as used herein generally refers to the strength of non-covalent binding. A lower KD signifies increased binding affinity. Binding between an antibody and antigen have an “affinity” that can be described by the dissociation constant (KD). An antibody or fragment thereof, including for example. Fab and Fv, can have much higher affinity for a biological target molecule than it has for an unrelated amino acid sequence, for example, at least 1, 2, 3, 4, 5, 6, 8, 10, 20, 40, 60, 100 or 1000 fold greater. Affinity of an antibody or a portion or fragment thereof to a biological target molecule can be, for example, from about 100 nanomolar (nM) to about 0.1 nM, from about 100 nM to about 1 picomolar (pM), or from about 100 nM to about 1 femtomolar (fM), or more.

The term “comprise” has its customary meaning, generally used in the sense of include, that is to say permitting the presence of one or more features or components.

The term “consisting essentially of” has its customary meaning and, for example, refers to a product, such as a peptide sequence, of a defined number of residues which is not covalently attached to a larger product. The disclosure permits other embodiments substituting “consisting essentially of” for “comprising” or “including.”

The term “oligonucleotide,” as used herein in referring to a probe of use the present disclosure, is defined as a molecule comprised of two or more ribonucleotides, preferably more than three. Its exact size will depend upon many factors which, in turn, depend upon the ultimate function and use of the oligonucleotide. The term “primer” as used herein refers to an oligonucleotide, which is capable of acting as a point of initiation of synthesis when placed under conditions in which synthesis of a primer extension product, which is complementary to a nucleic acid strand, is induced, i.e., in the presence of nucleotides and an inducing agent such as a DNA polymerase and at a suitable temperature and pH. The primer may be either single-stranded or double-stranded and must be sufficiently long to prime the synthesis of the desired extension product in the presence of the inducing agent. The exact length of the primer will depend upon many factors, including temperature, source of primer and use of the method. For example, for diagnostic applications, depending on the complexity of the target sequence, the oligonucleotide primer typically contains 15-25 or more nucleotides, although it may contain fewer nucleotides.

As used herein. “pg” means picogram, “ng” means nanogram, “ug” or “μg” mean microgram, “mg” means milligram, “ul” or “μl” mean microliter, “ml” means milliliter, “l” means liter.

An aspect of the disclosure features magnetic particles that capture biological target molecules for cryo-electron microscope imaging. The magnetic particles include paramagnetic beads. At least two spacer modules extend from a periphery of the paramagnetic beads including a first spacer module and a second spacer module. The first spacer module binds the paramagnetic beads and the second spacer module is located outwardly of the first spacer module and binds the first spacer module. The first spacer module includes first spacer proteins and the second spacer module includes second spacer proteins. A capture module is linked to an outer location of the at least two spacer modules. The capture module includes capture proteins that are adapted to capture biological target molecules by having affinity to the biological target molecules. The at least two spacer modules are arranged so that a combined length of the at least two spacer modules locates the capture module a distance that is spaced from the paramagnetic beads.

In an embodiment the distance by which the capture module is spaced from the paramagnetic beads is at least about 30 nm, at least about 40 nm, at least about 50 nm, at least about 60 nm, at least about 70 nm or at least about 80 nm.

In an embodiment the paramagnetic beads are not more than 100 nm in size, not more than 80 nm in size, not more than 60 nm in size, or not more than 50 nm in size.

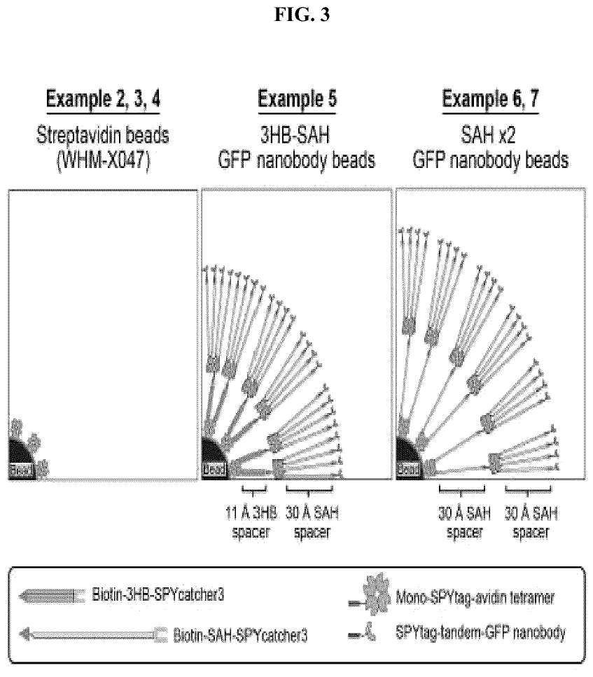

There are three exemplary designs of the magnetic particles of this disclosure. In the first design (FIG. 3, second panel from the left side), the magnetic particles include 3HB-30 nm-SAH-GFP nanobody. Throughout this disclosure the spacer proteins are referred to with regards to their length (e.g., 30 nm-SAH). This is an approximation of size and it should be apparent that this disclosure covers a variety of other sizes of spacer proteins and that the embodiments should not be limited to a particular size of spacer protein (even when a size is indicated in the text or drawings). In the second design (FIG. 3, third panel from the left side), the magnetic particles include 30 nm-SAH×2-GFP nanobody. In the third design (FIG. 3, far right panel), the magnetic particles include 3HB-60 nm-SAH-GFP nanobody.

In the first design of magnetic particles including 3HB-30 nm-SAH-GFP nanobody (FIG. 3, second panel from the left) the first spacer proteins include monomeric triple helical bundle (3HB) proteins and the second spacer proteins include single alpha helix (SAH) proteins. The first spacer proteins and the second spacer proteins can be linked end to end and together can form rays extending from the paramagnetic beads. Streptavidin binds the paramagnetic beads. The biotinylated 3HB proteins of the first spacer module can be about 11 nm in length, for example. The first spacer proteins include at their radially outer portion, a SPYcatcher003 moiety. The 3HB proteins are biotinylated at their radially inner location, for example, by way of a cysteine residue near the N terminus that is biotinylated. The second spacer module includes biotinylated single alpha helix (SAH) proteins that can be about 30 nm in length, for example. The SAH proteins are biotinylated at their radially inner location, for example, by way of a cysteine residue near the N terminus that is biotinylated. The SAH polypeptides of the second design, and the 3HB polypeptides and the SAH polypeptides of the third design, can also be biotinylated in this fashion. The second spacer module includes at its radially outer portion, a SPYcatcher003 moiety. The streptavidin on the beads binds biotin of the biotinylated 3HB proteins of the first spacer module. A mono-SPY-tagged avidin tetramer is linked to an outer end portion of the biotinylated 3HB proteins of the first spacer module and binds biotin of multiple of the biotinylated SAH proteins of the second spacer module. This enables more than one SAH polypeptide of the second module to be bound to each mono-SPY-tagged avidin tetramer. The SPYTAG003 moiety of the mono-SPY-tagged avidin tetramer is bonded to the SPYcatcher003 moiety of the first spacer proteins.

In the second design of magnetic particles including 30 nm-SAH×2 GFP nanobody (FIG. 3, third panel from the left), both of the first spacer proteins and the second spacer proteins are single alpha helix (SAH) proteins. The first spacer proteins of the first module and the second spacer proteins of the second module can be linked end to end and together can form rays extending from the paramagnetic beads. Streptavidin binds the paramagnetic beads. The first spacer module includes biotinylated SAH proteins, which can be about 30 nm in length, for example. The biotin of the first spacer proteins is located at a radially inner location of the first module. The first spacer module includes at its radially outer portion, a SPYcatcher003 moiety. The second spacer module includes biotinylated single alpha helix (SAH) proteins that can also be about 30 nm in length, for example. The streptavidin on the beads binds biotin of the biotinylated SAH proteins of the first spacer module. A mono-SPY-tagged avidin tetramer is linked to an outer end portion of the biotinylated SAH proteins of the first spacer module and binds biotin of multiple of the biotinylated SAH proteins of the second spacer module. This enables more than one SAH polypeptide of the second module to be bound to each avidin tetramer. The SPYtag003moiety of the mono-SPY-tagged avidin tetramer is bonded to the SPYcatcher003 moiety of the first spacer proteins.

In the third design of magnetic particles including 3HB-60 nm-SAH-GFP nanobody (FIG. 3, far right panel) first spacer proteins comprise monomeric triple helical bundle (3HB) proteins and second spacer proteins comprise 60 nm single alpha helix (SAH) proteins. The first spacer proteins and the second spacer proteins can be linked end to end and together can form rays extending from the paramagnetic beads. Streptavidin binds the paramagnetic beads. The first spacer module includes biotinylated monomeric triple helical bundle (3HB) first spacer proteins, which can be about 11 nm in length, for example. A radially inner portion of the first spacer proteins is biotinylated. At a radially outer portion of the first spacer proteins is a SPYcatcher003 moiety. The second spacer module includes biotinylated single alpha helix (SAH) proteins that can be about 60 nm in length, for example. A radially inner portion of the SAH second spacer proteins is biotinylated. At a radially outer portion of the SAH second spacer proteins is a SPYcatcher003 moiety. The streptavidin binds biotin of the biotinylated 3HB proteins of the first spacer module. A mono-SPY-tagged avidin tetramer is linked to an outer end portion of the biotinylated 3HB proteins of the first spacer module and binds biotin of multiple of the biotinylated 60 nm SAH proteins of the second spacer module. This enables more than one 60 nm SAH polypeptide of the second module to be bound to each mono-SPY-tagged avidin tetramer. The SPYtag003moiety of the mono-SPY-tagged avidin tetramer is bonded to the SPYcatcher003 moiety of the first spacer proteins.

In all three exemplary magnetic particle designs the capture modules that bond to the second spacer proteins can have a variety of components and can capture a variety of biological target molecules by having affinity to the biological target molecules. In an embodiment the capture module is adapted to capture at least one of the biological target molecules selected from at least one of a protein. RNA or DNA, and portions or fragments thereof. In an embodiment the capture module includes an antibody, antibody portion or antibody fragment. In an embodiment the capture module includes at least one of a nanobody, scFv or Fab.

In an embodiment the capture module has affinity for nucleic acid, DNA, RNA, protein or a fragment or portion thereof, and combinations thereof as the biological target molecules. In an embodiment the capture module has affinity for a protein or a tag of the protein. In an embodiment the at least one nanobody, scFv or Fab has affinity for a histone or a tag of the histone. In an embodiment the at least one nanobody, scFv or Fab has affinity for a protein or a tag of the protein, and the protein binds to DNA, the protein being one of the biological target molecules. In an embodiment the at least one nanobody, scFv or Fab has affinity for a protein or a tag of the protein, and the protein is associated with at least one other protein that binds to DNA, the protein being one of the biological target molecules. It should be appreciated that reference to DNA and RNA includes molecules of any length, including DNA being investigated after digesting chromatin.

More specifically, in the exemplary embodiments of the three designs, in one example the capture module is a nanobody comprising a sequence of SPYtag-GFP enhancer-linker-LaG16 that is adapted to bind GFP. The SPYtag003moiety is located near an end portion of the nanobody and bonds the SPYcatcher003 moiety at an outer end portion of the second module in any of the three designs above. In the first design the SPYtag003moiety of the capture module bonds the SPYcatcher003 moiety at the radially outer end portion of the second module. 30 nm-SAH protein. In the second design the SPYtag003moiety of the capture module bonds the SPYcatcher003 moiety at an outer end portion of the second module, 30 nm-SAH protein. In the third design the SPYtag003moiety of the capture module bonds the SPYcatcher003 moiety at an outer end portion of the second module, 60 nm-SAH protein.

Another aspect of the disclosure is a method of using cryo-electron microcopy to image a target molecule including the following steps. Any of the magnetic particles disclosed herein are provided including, but not limited to, the magnetic particles of any of the three designs shown in FIG. 3. The magnetic particles are mixed in a liquid with biological target molecules to capture the target molecules by affinity that the capture proteins have to the biological target molecules. A sheet of graphene is optionally placed onto an electron microscope (EM) grid. The EM grid has a plurality of holes as is known to those of ordinary skill in the art. A liquid including the magnetic particles with captured biological target molecules is applied onto the graphene sheet on the EM grid. The graphene sheet can avoid or lessen the effect of proteins as biological target molecules being exposed to an air-water interface located in the holes of the grid. The biological target molecules are concentrated in the liquid on the electron microscope grid by applying a magnetic field to the magnetic particles with captured biological target molecules. The EM grid is positioned over the magnet for this purpose. A portion of the liquid is removed from the EM grid, for example, by blotting with filter paper. The EM grid containing the magnetic particles and the captured biological target molecules is vitrified using a process known to persons of ordinary skill in the art. EM imaging of the target molecules is conducted. The method enables single particle analysis of the biological target molecules.

In an embodiment the capture module is a protein affinity purification system including at least one of Protein A, Strep-Tactin, calmodulin binding protein, single stranded DNA, double stranded DNA, single stranded RNA, double stranded RNA. SPYcatcher, streptavidin, streptavidin that binds NTA-biotin, streptavidin that binds biotinylated glutathione, streptavidin that binds Tris-NTA-biotin, streptavidin that binds Halotag biotin ligand, streptavidin that binds chitosan-biotin, or streptavidin that binds SNAP-biotin.

In another embodiment, the capture module is a DNA/RNA sequence-based affinity system including at least one of a nuclease-dead CRISPR-associated protein 9 (dCas9) attached with single guide RNA (sgRNA), zinc-finger proteins (ZFPs), transcription activator-like (TAL) proteins, PNA. LNA, single stranded DNA, single stranded RNA, or morpholino.

In an embodiment of the method the capture module includes an antibody, antibody portion or antibody fragment. In an embodiment of the method the capture module includes at least one of a nanobody, scFv or Fab. In an embodiment of the method the capture module has an affinity for a protein or a tag of the protein that forms a part of the biological target molecules.

In an embodiment of the method the at least one of a nanobody, scFv or Fab has affinity for a tag of a linker histone, the linker histone being bound on a nucleosome. The linker histone bound nucleosome is captured and EM imaging is conducted on at least one of the nucleosome and the captured histone as the target molecules. In an embodiment the method includes capturing in vitro reconstituted recombinant linker histone-bound nucleosomes. In an embodiment the method includes capturing recombinant linker histone-bound nucleosomes, which are isolated from chromosomes in a cellular environment.

In an embodiment the method uses GFP tagged Xenopus laevis H1.8 as the linker histone. The nanobody has an affinity for the GFP tag of this linker histone. When the GFP-H1.8 linker histone binds to a nucleosome, the nanobody having affinity for GFP, which is part of the capture module, enables the magnetic particles to capture the linker histone H1.8 and bound nucleosome. This enables cryo-EM imaging and single particle analysis of the linker histone H1.8 and nucleosome.

In an embodiment of the method the tag is at least one of a GFP tag. Myc tag. HA tag. V5-tag, CD tag, or FLAG tag, and combinations thereof.

In another embodiment of the method the tag is a peptide and protein affinity tag including at least one of SPYtag. CBP-tag. GST-tag, poly His-tag, SNAP-tag, CDB tag, Halo tag, Avitag. S-tag, or Strep-tag, and combinations thereof.

In an embodiment of the method at least one nanobody, scFv or Fab has affinity for a protein or a tag of the protein, the protein being associated with at least one other protein that bind to DNA. The protein is one of the biological target molecules. The protein is captured and EM imaging of the protein is conducted.

In an embodiment the method includes capturing a target molecule selected from at least one of a protein. RNA or DNA, and portions or fragments thereof and conducting EM imaging thereof.

In an embodiment of the method the biological target molecules are captured while present in a solution in a concentration of 34 nM (Examples 2-4), 100 nM (Examples 5, 6), 1 nM, 5 nM or 10 nM (Example 7), or 1.6 nM (Example 8). For example, protein and DNA as the biological target molecules can be recombinant H1.8-GFP bound nucleosome in a solution in a concentration of 1.6 nM (Example 8), which are captured by the magnetic particles of this disclosure.

Another aspect of the disclosure is a method of making magnetic particles for capturing a biological target molecule for cryo-electron microscope imaging, including the following steps. Paramagnetic beads conjugated with streptavidin are provided. Biotin-3HB-SPYcatcher003 proteins (first and third designs of FIG. 3) or biotin-SAH-SPYcatcher003 proteins (second design of FIG. 3) are added to the paramagnetic beads conjugated with streptavidin. The biotin of the biotin-3HB-SPYcatcher003 proteins binds the streptavidin on the beads (first design or third design of FIG. 3), or the biotin of the biotin-SAH-SPYcatcher003 proteins binds the streptavidin on the beads (second design of FIG. 3), attaching a first spacer module. Mono-SPY-tagged avidin tetramer proteins are added to the first spacer module. A SPYtag003 moiety of the mono-SPY-tagged avidin tetramer proteins bonds the SPYcatcher003 moiety of the biotin-3HB-SPYcatcher003 first spacer proteins (first design or third design of FIG. 3). Alternatively, a SPYtag003moiety of the mono-SPY-tagged avidin tetramer proteins bonds the SPYcatcher003 moiety of the biotin-SAH-SPYcatcher003 first spacer proteins (second design of FIG. 3). Biotinylated second module proteins including SAH and SPYcatcher003 moieties (e.g., 30 nm-biotin-SAH-SPYcatcher003 proteins of the first and second designs; or 60 nm biotin-SAH-SPYcatcher003 proteins of the third design) are added to the mono-SPY-tagged avidin tetramer proteins bonded to the first spacer module, to attach the second spacer module proteins. Biotin of the second spacer module proteins binds to the avidin of the mono-SPY-tagged avidin tetramer proteins.

In an embodiment of the method at least one buffer is present in any one or more of steps a)-d).

An embodiment of the method includes e) providing a capture module including a SPYtag003moiety and at least one capturing polypeptide that has an affinity to a biological target molecule, adding the capture module to the second module, wherein the SPYtag003moiety of the capture module bonds to the SPYcatcher003 moiety of the second spacer proteins of the second module.

Another aspect of the disclosure is a combination product including magnetic particles that capture a biological target molecule for cryo-electron microscope imaging including:
a) paramagnetic beads conjugated with streptavidin;
b) optional biotin-3HB-SPYcatcher003 proteins;
c) biotinylated proteins including SAH and SPYcatcher003 moieties;
d) mono-SPY-tagged avidin tetramer proteins; and
e) a capture module comprising a SPYtag003 moiety and at least one capturing polypeptide that is adapted to have affinity to a biological target molecule. The combination product can be provided with some or all of the components a)-e) and other components so that users can have all of the materials in one convenient item needed for conducting cryo-EM imaging of biological target molecules of interest. In an embodiment of the combination product at least two of components a), b), c), d) and e) are provided in separate packaging. For example, the combination product can include separate packaging for components a)-e), each provided in a suitable liquid such as a buffer. The combination product should be stored at a suitably cool temperature.

The combination product component b) can be present for making the first and third designs and omitted for making the second design.

The combination product component c) can include 30 nm-biotin-SAH-SPYcatcher003 proteins for making the first and second designs; or 60 nm biotin-SAH-SPYcatcher003 proteins for making the third design.

The combination product can include all of the components needed to make any of the three designs. It can include component b) for making the first and third designs which is not needed to make the second design. It can also include component c1) 30 nm-biotin-SAH-SPYcatcher003 proteins for making the first and second designs; and component c2) 60 nm biotin-SAH-SPYcatcher003 proteins for making the third design.

In an embodiment, the combination product includes at least one buffer containing one or more of components a)-e).

The disclosure now refers to Examples for more specific features, which should not be used to unduly limit the embodiments of the disclosure.

### Example 1: Methods and Materials

This example describes the general methods and materials used in Examples 2-8.

### Protein Purification

Biotin-3HB-SPYcatcher003 is bacterially expressed and purified using pQE80-His₁₄-bdSUMO-Cys-3HB-SPYcatcher003. To build the plasmid, pQE80 derivative vector encoding N-terminal His-tag encoding is amplified by PCR using pSF1389 [Addgene plasmid #104962] (Frey and Görlich, 2014). gBlock DNAs encoding Brachypodium distachyon SUMO (bdSUMO) (Frey and Görlich, 2014) and computationally designed monomeric three-helix bundle (Huang et al., 2014) are synthesized by IDT and used as a PCR template. DNA encoding SPYcatcher003 is amplified using pSpyCatcher003 [Addgene plasmid #133447] (Keeble et al., 2019) as a PCR template. DNA fragments are assembled by the Gibson assembly method (Gibson et al . . . 2009). E. Coli Rosetta (DE3) cells expressing His₁₄-bdSUMO-Cys-3HB-SPYcatcher003 by 1 mM isopropyl-β-D-thiogalactopyranoside (IPTG) induction at 25° C. are resuspended with 100 ml buffer A (8 mM Na₂HPO₄, 2 mM KH₂PO₄, 537 mM NaCl. 2.7 mM KCl, 10% glycerol. 2 mM 2-mercaptoethanol, 1 mM PMSF, 20 mM imidazole with 1× complete Protease Inhibitor Cocktail (Roche)) are disrupted by sonication. The soluble fraction is collected by centrifugation at 20,000 rpm (46,502 rcf) at 4° C. for 30 min using 45Ti rotor in Optima L80 (Beckman Coulter) and mixed with Ni-NTA agarose beads (Qiagen). Protein-bound Ni-NTA agarose beads are packed into an Econo-column (bio-rad) and washed with 170 column volume (CV) of buffer B (8 mM Na₂HPO₄, 2 mM KH₂PO₄, 937 mM NaCl. 2.7 mM KCl, 10% glycerol, 2 mM 2-mercaptoethanol, 1 mM PMSF, 40 mM imidazole with 1× complete Protease Inhibitor Cocktail (Roche), [pH 7.4]). The beads are further washed with 33 CV of Phosphate-Buffered Saline (PBS: 8 mM Na₂HPO₄, 2 mM KH₂PO₄, 137 mM NaCl, 2.7 mM KCl [pH 7.4]) containing additional 5% glycerol to remove 2-mercaptocthanol. His₁₄-SUMO-tag is cleaved by incubating overnight at 4° C. with N-terminal His-tagged SENP1 protease that is expressed and purified with the previously described method using pSF1389 [Addgene plasmid #104962] (Frey and Görlich, 2014). Ni-NTA agarose beads that bound the cleaved His₁₄-bdSUMO-tag and His₁₄-SENP1 are filtered out using Econo-column (bio-rad). The cleaved 3HB-SPYcatcher003 with cysteine residue on N-terminal is concentrated by Amicon 30K (Millipore), mixed with EZ-link Maleimide-PEG2-Biotin (Thermo A39261), and placed at 4° C. overnight. Biotinylated 3HB-SPYcatcher003 is dialyzed overnight against PBS at 4° C. The dialyzed Biotin-3HB-SPYcatcher003 is further purified though a Hi-load Superdex75 16/600 column (Cytiva) and stored at −20° C. in PBS containing 47.5% glycerol. Listed below is the DNA sequence of pQE80-His₁₄-bdSUMO-Cys-3HB-SPYcatcher003 and the amino acid sequence of Biotin-3HB-SPYcatcher003. The cysteine residue on N-terminal is the biotinylation site.

pQE80-His14-bdSUMO-Cys-3HB-SPYcatcher003
(SEQ ID NO: 1)
TTAAGGGATTTTGGTCATGACATTAACCTATAAAAATAGGCGTATCACGAGGCCCTTTCGTCT
TCACCTCGAGAAATCATAAAAAATTTATTTGCTTTGTGAGCGGATAACAATTATAATAGATTC
AATTGTGAGCGGATAACAATTTCACACAGAATTCATTAAAGAGGAGAAATTAACCATGAGCAA
GCATCACCATCATTCAGGCCATCACCATACCGGACACCACCATCATTCAGGCAGTCATCACCA
TACCGGCGAGAACCTGTATTTTCAGGGTTCAGCTGCAGGCGGTGAAGAGGATAAAAAGCCAGC
CGGAGGGGAAGGTGGTGGTGCTCATATTAACTTAAAGGTTAAAGGACAGGATGGCAACGAGGT
GTTCTTCCGCATTAAGCGCAGTACGCAACTGAAAAAACTGATGAACGCCTACTGTGACCGTCA
ATCAGTTGACATGACTGCCATTGCGTTCTTATTTGACGGTCGCCGCCTGCGTGCAGAACAGAC
ACCAGACGAGTTAGAAATGGAAGATGGAGATGAGATCGATGCGATGTTGCATCAGACAGGCGG
TGCGGGTACCTGCGGTACCCTGTATAAACAAATGGTGCAGGAGCTGGAAAAAGCCCGTGACCG
CATGGAGAAGTTATACAAAGAAATGGTGGAGTTAATCCAGAAAGCGATCGAACTGATGCGTAA
AATTTTCCAGGAAGTAAAACAGGAGGTTGAGAAGGCCATTGAGGAGATGAAAAAATTGTACGA
TGAAGCTAAAAAAAAGATTGAGCAAATGATTCAACAAATTAAGCAGGGAGGCGATAAACAGAA
AATGGAAGAGTTACTTAAGCGTGCCAAAGAAGAGATGAAAAAAGTCAAAGACAAGATGGAAAA
ATTATTAGAAAAGTTAAAGCAAATCATGCAGGAAGCCAAACAAAAGATGGAGAAGCTGTTAAA
ACAATTAAAGGAAGAGATGAAAAAAATGAAAGAAAAAATGGAAAAGTTATTGAAGGAAATGAA
GCAGCGCATGGAGGAAGTCAAAAAGAAGATGGACGGGGATGATGAATTGTTAGAAAAGATTAA
AAAAAATATCGATGATTTAAAGAAAATTGCCGAGGATTTAATCAAAAAAGCCGAGGAAAACAT
CAAGGAGGCCAAAAAGATCGCAGAGCAGCTGGTTAAGCGCGCAAAACAATTAATCGAAAAAGC
AAAGCAAGTAGCAGAGGAGCTTATTAAGAAGATCTTGCAATTGATTGAAAAAGCAAAGGAAAT
TGCTGAAAAGGTTCTGAAAGGCGGCAGTGCCATGGTAACCACCTTATCAGGTTTATCAGGTGA
GCAAGGTCCGTCCGGTGATATGACAACTGAAGAAGATAGTGCTACCCATATTAAATTCTCAAA
ACGTGATGAGGACGGCCGTGAGTTAGCTGGTGCAACTATGGAGTTGCGTGATTCATCTGGTAA
AACTATTAGTACATGGATTTCAGATGGACATGTGAAGGATTTCTACCTGTATCCAGGAAAATA
TACATTTGTCGAAACCGCAGCACCAGACGGTTATGAGGTAGCAACTCCAATTGAATTTACAGT
TAATGAGGACGGTCAGGTTACTGTAGATGGTGAAGCAACTGAAGGTGACGCTCATACTGGATC
CAGTGGTAGCTAGTAAGCTTAATTAGCTGAGCTTGGACTCCTGTTGATAGATCCAGTAATGAC
CTCAGAACTCCATCTGGATTTGTTCAGAACGCTCGGTTGCCGCCGGGCGTTTTTTATTGGTGA
GAATCCAAGCTAGCCATGAAAATAAACTGTCTGCTTACATAAACAGTAATACAAGGGGTGTTA
TGAGCCATATTCAACGGGAAACGTCTTGCTCTAGGCCGCGATTAAATTCCAACATGGATGCTG
ATTTATATGGGTATAAATGGGCTCGCGATAATGTCGGGCAATCAGGTGCGACAATCTATCGAT
TGTATGGGAAGCCCGATGCGCCAGAGTTGTTTCTGAAACATGGCAAAGGTAGCGTTGCCAATG
ATGTTACAGATGAGATGGTCAGACTAAACTGGCTGACGGAATTTATGCCTCTTCCGACCATCA
AGCATTTTATCCGTACTCCTGATGATGCTTGGTTACTCACGACTGCGATCCCCGGCAAAACAG
CATTCCAGGTATTAGAAGAATATCCTGATTCAGGTGAAAATATTGTTGATGCGCTGGCAGTGT
TCCTGCGCCGGTTGCATTCGATTCCTGTTTGTAATTGTCCTTTTAACAGCGATCGCGTATTTC
GTCTCGCTCAGGCGCAATCACGAATGAATAACGGTTTGGTTGATGCGAGTGATTTTGATGACG
AGCGTAATGGCTGGCCTGTTGAACAAGTCTGGAAAGAAATGCACAAACTTTTGCCATTCTCAC
CGGATTCAGTCGTCACTCATGGTGATTTCTCACTTGATAACCTTATTTTTGACGAGGGGAAAT
TAATAGGTTGTATTGATGTTGGACGAGTCGGAATCGCAGACCGATACCAGGATCTTGCCATCC
TATGGAACTGCCTCGGTGAGTTTTCTCCTTCATTACAGAAACGGCTTTTTCAAAAATATGGTA
TTGATAATCCTGATATGAATAAATTGCAGTTTCATTTGATGCTCGATGAGTTTTTCTAAGAAT
TAATTCATGGGCAAATATTATACGCAAGGCGACAAGGTGCTGATGCCGCTGGCGATTCAGGTT
CATCATGCCGTTTGTGATGGCTTCCATGTCGGCAGAATGCTTAATGAATTACAACAGTACTGC
GATGAGTGGCAGGGCGGGGCGTAATTTTTTTAAGGCAGTTATTGGTGCCCTTAAACGCCTGGG
GTAATGACTCTCTAGCTTGAGGCATCAAATAAAACGAAAGGCTCAGTCGAAAGACTGGGCCTT
TCGTTTTATCTGTTGTTTGTCGGTGAACGCTCTCCTGAGTAGGACAAATCCGCCCTCTAGATT
ACGTGCAGTCGATGATAAGCTGTCAAACATGAGAATTGTGCCTAATGAGTGAGCTAACTTACA
TTAATTGCGTTGCGCTCACTGCCCGCTTTCCAGTCGGGAAACCTGTCGTGCCAGCTGCATTAA
TGAATCGGCCAACGCGCGGGGAGAGGCGGTTTGCGTATTGGGCGCCAGGGTGGTTTTTCTTTT
CACCAGTGAGACGGGCAACAGCTGATTGCCCTTCACCGCCTGGCCCTGAGAGAGTTGCAGCAA
GCGGTCCACGCTGGTTTGCCCCAGCAGGCGAAAATCCTGTTTGATGGTGGTTAACGGCGGGAT
ATAACATGAGCTGTCTTCGGTATCGTCGTATCCCACTACCGAGATATCCGCACCAACGCGCAG
CCCGGACTCGGTAATGGCGCGCATTGCGCCCAGCGCCATCTGATCGTTGGCAACCAGCATCGC
AGTGGGAACGATGCCCTCATTCAGCATTTGCATGGTTTGTTGAAAACCGGACATGGCACTCCA
GTCGCCTTCCCGTTCCGCTATCGGCTGAATTTGATTGCGAGTGAGATATTTATGCCAGCCAGC
CAGACGCAGACGCGCCGAGACAGAACTTAATGGGCCCGCTAACAGCGCGATTTGCTGGTGACC
CAATGCGACCAGATGCTCCACGCCCAGTCGCGTACCGTCTTCATGGGAGAAAATAATACTGTT
GATGGGTGTCTGGTCAGAGACATCAAGAAATAACGCCGGAACATTAGTGCAGGCAGCTTCCAC
AGCAATGGCATCCTGGTCATCCAGCGGATAGTTAATGATCAGCCCACTGACGCGTTGCGCGAG
AAGATTGTGCACCGCCGCTTTACAGGCTTCGACGCCGCTTCGTTCTACCATCGACACCACCAC
GCTGGCACCCAGTTGATCGGCGCGAGATTTAATCGCCGCGACAATTTGCGACGGCGCGTGCAG
GGCCAGACTGGAGGTGGCAACGCCAATCAGCAACGACTGTTTGCCCGCCAGTTGTTGTGCCAC
GCGGTTGGGAATGTAATTCAGCTCCGCCATCGCCGCTTCCACTTTTTCCCGCGTTTTCGCAGA
AACGTGGCTGGCCTGGTTCACCACGCGGGAAACGGTCTGATAAGAGACACCGGCATACTCTGC
GACATCGTATAACGTTACTGGTTTCACATTCACCACCCTGAATTGACTCTCTTCCGGGCGCTA
TCATGCCATACCGCGAAAGGTTTTGCACCATTCGATGGTGTCGGAATTTCGGGCAGCGTTGGG
TCCTGGCCACGGGTGCGCATGATCTAGAGCTGCCTCGCGCGTTTCGGTGATGACGGTGAAAAC
CTCTGACACATGCAGCTCCCGGAGACGGTCACAGCTTGTCTGTAAGCGGATGCCGGGAGCAGA
CAAGCCCGTCAGGGCGCGTCAGCGGGTGTTGGCGGGTGTCGGGGCGCAGCCATGACCCAGTCA
CGTAGCGATAGCGGAGTGTATACTGGCTTAACTATGCGGCATCAGAGCAGATTGTACTGAGAG
TGCACCATATGCGGTGTGAAATACCGCACAGATGCGTAAGGAGAAAATACCGCATCAGGCGCT
CTTCCGCTTCCTCGCTCACTGACTCGCTGCGCTCGGTCGTTCGGCTGCGGCGAGCGGTATCAG
CTCACTCAAAGGCGGTAATACGGTTATCCACAGAATCAGGGGATAACGCAGGAAAGAACATGT
GAGCAAAAGGCCAGCAAAAGGCCAGGAACCGTAAAAAGGCCGCGTTGCTGGCGTTTTTCCATA
GGCTCCGCCCCCCTGACGAGCATCACAAAAATCGACGCTCAAGTCAGAGGTGGCGAAACCCGA
CAGGACTATAAAGATACCAGGCGTTTCCCCCTGGAAGCTCCCTCGTGCGCTCTCCTGTTCCGA
CCCTGCCGCTTACCGGATACCTGTCCGCCTTTCTCCCTTCGGGAAGCGTGGCGCTTTCTCATA
GCTCACGCTGTAGGTATCTCAGTTCGGTGTAGGTCGTTCGCTCCAAGCTGGGCTGTGTGCACG
AACCCCCCGTTCAGCCCGACCGCTGCGCCTTATCCGGTAACTATCGTCTTGAGTCCAACCCGG
TAAGACACGACTTATCGCCACTGGCAGCAGCCACTGGTAACAGGATTAGCAGAGCGAGGTATG
TAGGCGGTGCTACAGAGTTCTTGAAGTGGTGGCCTAACTACGGCTACACTAGAAGGACAGTAT
TTGGTATCTGCGCTCTGCTGAAGCCAGTTACCTTCGGAAAAAGAGTTGGTAGCTCTTGATCCG
GCAAACAAACCACCGCTGGTAGCGGTGGTTTTTTTGTTTGCAAGCAGCAGATTACGCGCAGAA
AAAAAGGATCTCAAGAAGATCCTTTGATCTTTTCTACGGGGTCTGACGCTCAGTGGAACGAAA
ACTCACG
>Biotin-3HB-SPYcatcher003
(SEQ ID NO: 2)
AGTC*GTLYKQMVQELEKARDRMEKLYKEMVELIQKAIELMRKIFQEVKQEVEKAIEEMKKLY
DEAKKKIEQMIQQIKQGGDKQKMEELLKRAKEEMKKVKDKMEKLLEKLKQIMQEAKQKMEKLL
KQLKEEMKKMKEKMEKLLKEMKQRMEEVKKKMDGDDELLEKIKKNIDDLKKIAEDLIKKAEEN
IKEAKKIAEQLVKRAKQLIEKAKQVAEELIKKILQLIEKAKEIAEKVLKGGSAMVTTLSGLSG
EQGPSGDMTTEEDSATHIKFSKRDEDGRELAGATMELRDSSGKTISTWISDGHVKDFYLYPGK
YTFVETAAPDGYEVATPIEFTVNEDGQVTVDGEATEGDAHTGSSGS
[C* biotinylated cysteine]

Biotin-30 nm-SAH-SPYcatcher003 is bacterially expressed and purified using pQE80-His₁₄-bdSUMO-Cys-30 nm-SAH-SPYcatcher003. DNA encoded 30 nm single alpha-helix from Trichomonas Vaginalis is amplified using pCDNA-FRT-FAK30 [Addgene plasmid #59121] (Sivaramakrishnan and Spudich, 2011) as a PCR template. E. Coli Rosetta (DE3) cells expressing His₁₄-bdSUMO-Cys-30 nm-SAH-SPYcatcher003 by 1 mM IPTG induction at 18° C. are resuspended with 100 ml buffer A and disrupted by sonication. A soluble fraction is collected by centrifugation at 20,000 rpm (46,502 rcf) at 4° C. for 30 min using 45Ti rotor in Optima L80 (Beckman Coulter) and applied to the HisTrap HP column (Cytiva). The column is washed with 4 column volume (CV) of buffer B. His₁₄-bdSUMO-Cys-30 nm-SAH-SPYcatcher003 is cluted from the HisTrap column by buffer D (8 mM Na₂HPO₄, 2 mM KH₂PO₄. 137 mM NaCl, 2.7 mM KCl, 5% glycerol, 200 mM imidazole [pH 7.4]). The eluted His₁₄-bdSUMO-Cys-30 nm-SAH-SPYcatcher003 is mixed with His₁₄-SENP1 and dialyzed against PBS containing 5% glycerol at 4° C. overnight. The dialyzed protein is mixed with Ni-NTA agarose beads (Qiagen), and the beads that bound the cleaved His₁₄-bdSUMO-tag and His₁₄-SENP1 are filtered out through an Econo-column (bio-rad). The cleaved 30 nm-SAH-SPYcatcher003 with a cysteine residue on N-terminal is concentrated with Amicon 30K (Millipore), mixed with EZ-link Maleimide-PEG2-Biotin (Thermo A39261), and placed overnight at 4° C. The biotinylated 30 nm-SAH-SPYcatcher003 is dialyzed against PBS at 4° C. overnight. The dialyzed Biotin-60 nm-SAH-SPYcatcher003 is purified through Hi-load Superdex200 16/600 column (Cytiva) and stored at −20° C. in PBS containing 47.5% glycerol. Listed below is the DNA sequence of pQE80-His₁₄-bdSUMO-Cys-30 nm-SAH-SPYcatcher003 and the amino acid sequence of Biotin-30 nm-SAH-SPYcatcher003. The cysteine residue on N-terminal is the biotinylated site.

>pQE80-His₁₄-bdSUMO-Cys-30 nm-SAH-SPYcatcher003 (SEQ ID NO: 3)

TTAAGGGATTTTGGTCATGACATTAACCTATAAAAATAGGCGTATCACGAGGCCCTT
TCGTCTTCACCTCGAGAAATCATAAAAAATTTATTTGCTTTGTGAGCGGATAACAAT
TATAATAGATTCAATTGTGAGCGGATAACAATTTCACACAGAATTCATTAAAGAGGA
GAAATTAACCATGAGCAAGCATCACCATCATTCAGGCCATCACCATACCGGACACC
ACCATCATTCAGGCAGTCATCACCATACCGGCGAGAACCTGTATTTTCAGGGTTCAG
CTGCAGGCGGTGAAGAGGATAAAAAGCCAGCCGGAGGGGAAGGTGGTGGTGCTCA
TATTAACTTAAAGGTTAAAGGACAGGATGGCAACGAGGTGTTCTTCCGCATTAAGCG
CAGTACGCAACTGAAAAAACTGATGAACGCCTACTGTGACCGTCAATCAGTTGACA
TGACTGCCATTGCGTTCTTATTTGACGGTCGCCGCCTGCGTGCAGAACAGACACCAG
ACGAGTTAGAAATGGAAGATGGAGATGAGATCGATGCGATGTTGCATCAGACAGGC
GGTGCGGGTACCTGCGGTACCCAGGGCGGAAGCGGAGAAGAGGAAGAGAAGAAGA
AAGAAGAGGAAGAAAAGAAACAAAAAGAAGAACAAGAAAGACTTGCAAAAGAAG
AGGCAGAGAGAAAACAAAAAGAAGAACAAGAAAGACTTGCAAAAGAAGAGGCAG
AGAGAAAACAAAAGGAGGAAGAAGAGAGAAAACAAAAGGAAGAAGAAGAGAGAA
AACAAAAGGAGGAAGAAGAAAGAAAATTAAAGGAGGAACAAGAAAGAAAAGCTG
CAGAAGAAAAGAAAGCTAAAGAAGAAGCTGAGAGAAAGGCTAAAGAAGAACAAG
AAAGGAAAGCTGAAGAAGAGAGAAAGAAGAAAGAAGAGGAAGAAAGACTTGAAA
GAGAAAGAAAAGAGAGAGAAGAACAAGAAAAGAAAGCCAAAGAAGAGGCAGAGA
GAATTGCAAAGTTAGAGGCTGAAAAGAAGGCAGAAGAAGAAAGAAAAGCCAAAGA
AGAAGAAGAGAGAAAAGCCAAAGAAGAAGAGGAAAGAAAGAAGAAAGAGGAGCA
AGAAAGACTTGCAAAAGAAAAGGAAGAAGCAGAAAGAAAAGCTGCAGAGGAAAA
GAAAGCTAAAGAAGAACAAGAAAGAAAAGAAAAGGAAGAAGCAGAAAGAAAACA
AAGAGGCTCTGGCGGCTCTGGCGGCGCCATGGTAACCACCTTATCAGGTTTATCAGG
TGAGCAAGGTCCGTCCGGTGATATGACAACTGAAGAAGATAGTGCTACCCATATTA
AATTCTCAAAACGTGATGAGGACGGCCGTGAGTTAGCTGGTGCAACTATGGAGTTG
CGTGATTCATCTGGTAAAACTATTAGTACATGGATTTCAGATGGACATGTGAAGGAT
TTCTACCTGTATCCAGGAAAATATACATTTGTCGAAACCGCAGCACCAGACGGTTAT
GAGGTAGCAACTCCAATTGAATTTACAGTTAATGAGGACGGTCAGGTTACTGTAGAT
GGTGAAGCAACTGAAGGTGACGCTCATACTGGATCCAGTGGTAGCTAGTAAGCTTA
ATTAGCTGAGCTTGGACTCCTGTTGATAGATCCAGTAATGACCTCAGAACTCCATCT
GGATTTGTTCAGAACGCTCGGTTGCCGCCGGGCGTTTTTTATTGGTGAGAATCCAAG
CTAGCCATGAAAATAAACTGTCTGCTTACATAAACAGTAATACAAGGGGTGTTATGA
GCCATATTCAACGGGAAACGTCTTGCTCTAGGCCGCGATTAAATTCCAACATGGATG
CTGATTTATATGGGTATAAATGGGCTCGCGATAATGTCGGGCAATCAGGTGCGACAA
TCTATCGATTGTATGGGAAGCCCGATGCGCCAGAGTTGTTTCTGAAACATGGCAAAG
GTAGCGTTGCCAATGATGTTACAGATGAGATGGTCAGACTAAACTGGCTGACGGAA
TTTATGCCTCTTCCGACCATCAAGCATTTTATCCGTACTCCTGATGATGCTTGGTTAC
TCACGACTGCGATCCCCGGCAAAACAGCATTCCAGGTATTAGAAGAATATCCTGATT
CAGGTGAAAATATTGTTGATGCGCTGGCAGTGTTCCTGCGCCGGTTGCATTCGATTC
CTGTTTGTAATTGTCCTTTTAACAGCGATCGCGTATTTCGTCTCGCTCAGGCGCAATC
ACGAATGAATAACGGTTTGGTTGATGCGAGTGATTTTGATGACGAGCGTAATGGCTG
GCCTGTTGAACAAGTCTGGAAAGAAATGCACAAACTTTTGCCATTCTCACCGGATTC
AGTCGTCACTCATGGTGATTTCTCACTTGATAACCTTATTTTTGACGAGGGGAAATTA
ATAGGTTGTATTGATGTTGGACGAGTCGGAATCGCAGACCGATACCAGGATCTTGCC
ATCCTATGGAACTGCCTCGGTGAGTTTTCTCCTTCATTACAGAAACGGCTTTTTCAAA
AATATGGTATTGATAATCCTGATATGAATAAATTGCAGTTTCATTTGATGCTCGATG
AGTTTTTCTAAGAATTAATTCATGGGCAAATATTATACGCAAGGCGACAAGGTGCTG
ATGCCGCTGGCGATTCAGGTTCATCATGCCGTTTGTGATGGCTTCCATGTCGGCAGA
ATGCTTAATGAATTACAACAGTACTGCGATGAGTGGCAGGGCGGGGCGTAATTTTTT
TAAGGCAGTTATTGGTGCCCTTAAACGCCTGGGGTAATGACTCTCTAGCTTGAGGCA
TCAAATAAAACGAAAGGCTCAGTCGAAAGACTGGGCCTTTCGTTTTATCTGTTGTTT
GTCGGTGAACGCTCTCCTGAGTAGGACAAATCCGCCCTCTAGATTACGTGCAGTCGA
TGATAAGCTGTCAAACATGAGAATTGTGCCTAATGAGTGAGCTAACTTACATTAATT
GCGTTGCGCTCACTGCCCGCTTTCCAGTCGGGAAACCTGTCGTGCCAGCTGCATTAA
TGAATCGGCCAACGCGCGGGGAGAGGCGGTTTGCGTATTGGGCGCCAGGGTGGTTT
TTCTTTTCACCAGTGAGACGGGCAACAGCTGATTGCCCTTCACCGCCTGGCCCTGAG
AGAGTTGCAGCAAGCGGTCCACGCTGGTTTGCCCCAGCAGGCGAAAATCCTGTTTGA
TGGTGGTTAACGGCGGGATATAACATGAGCTGTCTTCGGTATCGTCGTATCCCACTA
CCGAGATATCCGCACCAACGCGCAGCCCGGACTCGGTAATGGCGCGCATTGCGCCC
AGCGCCATCTGATCGTTGGCAACCAGCATCGCAGTGGGAACGATGCCCTCATTCAGC
ATTTGCATGGTTTGTTGAAAACCGGACATGGCACTCCAGTCGCCTTCCCGTTCCGCT
ATCGGCTGAATTTGATTGCGAGTGAGATATTTATGCCAGCCAGCCAGACGCAGACGC
GCCGAGACAGAACTTAATGGGCCCGCTAACAGCGCGATTTGCTGGTGACCCAATGC
GACCAGATGCTCCACGCCCAGTCGCGTACCGTCTTCATGGGAGAAAATAATACTGTT
GATGGGTGTCTGGTCAGAGACATCAAGAAATAACGCCGGAACATTAGTGCAGGCAG
CTTCCACAGCAATGGCATCCTGGTCATCCAGCGGATAGTTAATGATCAGCCCACTGA
CGCGTTGCGCGAGAAGATTGTGCACCGCCGCTTTACAGGCTTCGACGCCGCTTCGTT
CTACCATCGACACCACCACGCTGGCACCCAGTTGATCGGCGCGAGATTTAATCGCCG
CGACAATTTGCGACGGCGCGTGCAGGGCCAGACTGGAGGTGGCAACGCCAATCAGC
AACGACTGTTTGCCCGCCAGTTGTTGTGCCACGCGGTTGGGAATGTAATTCAGCTCC
GCCATCGCCGCTTCCACTTTTTCCCGCGTTTTCGCAGAAACGTGGCTGGCCTGGTTCA
CCACGCGGGAAACGGTCTGATAAGAGACACCGGCATACTCTGCGACATCGTATAAC
GTTACTGGTTTCACATTCACCACCCTGAATTGACTCTCTTCCGGGCGCTATCATGCCA
TACCGCGAAAGGTTTTGCACCATTCGATGGTGTCGGAATTTCGGGCAGCGTTGGGTC
CTGGCCACGGGTGCGCATGATCTAGAGCTGCCTCGCGCGTTTCGGTGATGACGGTGA
AAACCTCTGACACATGCAGCTCCCGGAGACGGTCACAGCTTGTCTGTAAGCGGATG
CCGGGAGCAGACAAGCCCGTCAGGGCGCGTCAGCGGGTGTTGGCGGGTGTCGGGGC
GCAGCCATGACCCAGTCACGTAGCGATAGCGGAGTGTATACTGGCTTAACTATGCG
GCATCAGAGCAGATTGTACTGAGAGTGCACCATATGCGGTGTGAAATACCGCACAG
ATGCGTAAGGAGAAAATACCGCATCAGGCGCTCTTCCGCTTCCTCGCTCACTGACTC
GCTGCGCTCGGTCGTTCGGCTGCGGCGAGCGGTATCAGCTCACTCAAAGGCGGTAAT
ACGGTTATCCACAGAATCAGGGGATAACGCAGGAAAGAACATGTGAGCAAAAGGC
CAGCAAAAGGCCAGGAACCGTAAAAAGGCCGCGTTGCTGGCGTTTTTCCATAGGCT
CCGCCCCCCTGACGAGCATCACAAAAATCGACGCTCAAGTCAGAGGTGGCGAAACC
CGACAGGACTATAAAGATACCAGGCGTTTCCCCCTGGAAGCTCCCTCGTGCGCTCTC
CTGTTCCGACCCTGCCGCTTACCGGATACCTGTCCGCCTTTCTCCCTTCGGGAAGCGT
GGCGCTTTCTCATAGCTCACGCTGTAGGTATCTCAGTTCGGTGTAGGTCGTTCGCTCC
AAGCTGGGCTGTGTGCACGAACCCCCCGTTCAGCCCGACCGCTGCGCCTTATCCGGT
AACTATCGTCTTGAGTCCAACCCGGTAAGACACGACTTATCGCCACTGGCAGCAGCC
ACTGGTAACAGGATTAGCAGAGCGAGGTATGTAGGCGGTGCTACAGAGTTCTTGAA
GTGGTGGCCTAACTACGGCTACACTAGAAGGACAGTATTTGGTATCTGCGCTCTGCT
GAAGCCAGTTACCTTCGGAAAAAGAGTTGGTAGCTCTTGATCCGGCAAACAAACCA
CCGCTGGTAGCGGTGGTTTTTTTGTTTGCAAGCAGCAGATTACGCGCAGAAAAAAAG
GATCTCAAGAAGATCCTTTGATCTTTTCTACGGGGTCTGACGCTCAGTGGAACGAAA
ACTCACG
>Biotin-30nm-SAH-SPYcatcher003
(SEQ ID NO: 4)
AGTC*GTQGGSGEEEEKKKEEEEKKQKEEQERLAKEEAERKQKEEQERLAKEEAERKQ
KEEEERKQKEEEERKQKEEEERKLKEEQERKAAEEKKAKEEAERKAKEEQERKAEEER
KKKEEEERLERERKEREEQEKKAKEEAERIAKLEAEKKAEEERKAKEEEERKAKEEEER
KKKEEQERLAKEKEEAERKAAEEKKAKEEQERKEKEEAERKQRGSGGSGGAMVTTLS
GLSGEQGPSGDMTTEEDSATHIKFSKRDEDGRELAGATMELRDSSGKTISTWISDGHVK
DFYLYPGKYTFVETAAPDGYEVATPIEFTVNEDGQVTVDGEATEGDAHTGSSGS
[C* biotinylated cysteine]

Biotin-60 nm-SAH-SPYcatcher003 is bacterially expressed and purified using pQE80-His 14-bdSUMO-Cys-60 nm-SAH-SPYcatcher003. To generate pQE80-His₁₄-bdSUMO-Cys-60 nm-SAH-SPYcatcher003, two plasmids, pQE80-His₁₄-bdSUMO-Cys-AflII-30 nm-SAH-SPYcatcher003 and pQE80-His₁₄-bdSUMO-Cys-30 nm-SAH-AflII-SPYcatcher003 that have an AflII recognition sequence before or after 30 nm single alpha-helix coding are prepared with KOD mutagenesis method using pQE80-His 14-bdSUMO-Cys-30 nm-SAH-SPYcatcher003 as a template. Both plasmids are treated with Xhol and AflII, and the longer fragment generated from pQE80-His 14-bdSUMO-Cys-AflII-30 nm-SAH-SPYcatcher003 that contains pQE80 vector backbone and 30 nm SAH and SPYcatcher003 coding DNA is isolated by cutting out the band from agarose electrophoresis gel. The shorter fragment generated from pQE80-His₁₄-bdSUMO-Cys-30 nm-SAH-AflII-SPYcatcher003 that contains His-tag, bdSUMO, and 30 nm SAH coding DNA is also isolated by cutting out the band from agarose electrophoresis gel. The fragments are ligated to make pQE80-His₁₄-bdSUMO-Cys-60 nm-SAH-SPYcatcher003. E. Coli Rosetta (DE3) cells cxpressing His 14-bdSUMO-Cys-60 nm-SAH-SPYcatcher003 by 1 mM IPTG induction at 18° C. are resuspended with 100 ml buffer A and disrupted by sonication. A soluble fraction is collected by centrifugation at 20,000 rpm (46,502 rcf) at 4° C. for 30 min using 45Ti rotor in Optima L80 (Beckman Coulter) and applied to the HisTrap HP column (Cytiva). The column is washed with 4 column volume (CV) of buffer B. His₁₄-bdSUMO-Cys-60 nm-SAH-SPYcatcher003 is eluted from the His Trap column by buffer D (8 mM Na₂HPO₄, 2 mM KH₂PO₄, 137 mM NaCl, 2.7 mM KCl. 5% glycerol. 200 mM imidazole [pH 7.4]). The eluted His₁₄-bdSUMO-Cys-60 nm-SAH-SPYcatcher003 is mixed with His₁₄-SENP1 and dialyzed against PBS containing 5% glycerol at 4° C. overnight. The dialyzed protein is applied to the HisTrap HP column (Cytiva) to remove the cleaved His₁₄-bdSUMO-tag and His₁₄-SENP1. The cleaved 60 nm-SAH-SPYcatcher003 is further purified through MonoQ 5/50 column (Cytiva). The purified 60 nm-SAH-SPYcatcher003 with a cysteine residue on N-terminal is concentrated with Amicon 10K (Millipore), mixed with EZ-link Maleimide-PEG2-Biotin (Thermo A39261), and placed overnight at 4° C. The biotinylated 60 nm-SAH-SPYcatcher003 is dialyzed against PBS at 4° C. overnight. The dialyzed Biotin-60 nm-SAH-SPYcatcher003 is purified through Hi-load Superdex200 16/600 column (Cytiva) and stored at −20° C. in PBS containing 47.5% glycerol. Listed below is the DNA sequence of pQE80-His₁₄-bdSUMO-Cys-60 nm-SAH-SPYcatcher003 and the amino acid sequence of Biotin-60 nm-SAH-SPYcatcher003. The cysteine residue on N-terminal is the biotinylated site.

>pQE80-His₁₄-bdSUMO-Cys-60nm-SAH-SPYcatcher003
(SEQ ID NO: 5)
TTAAGGGATTTTGGTCATGACATTAACCTATAAAAATAGGCGTATCACGAGGCCCTT
TCGTCTTCACCTCGAGAAATCATAAAAAATTTATTTGCTTTGTGAGCGGATAACAAT
TATAATAGATTCAATTGTGAGCGGATAACAATTTCACACAGAATTCATTAAAGAGGA
GAAATTAACCATGAGCAAGCATCACCATCATTCAGGCCATCACCATACCGGACACC
ACCATCATTCAGGCAGTCATCACCATACCGGCGAGAACCTGTATTTTCAGGGTTCAG
CTGCAGGCGGTGAAGAGGATAAAAAGCCAGCCGGAGGGGGAGGTGGTGGTGCTCA
TATTAACTTAAAGGTTAAAGGACAGGATGGCAACGAGGTGTTCTTCCGCATTAAGCG
CAGTACGCAACTGAAAAAACTGATGAACGCCTACTGTGACCGTCAATCAGTTGACA
TGACTGCCATTGCGTTCTTATTTGACGGTCGCCGCCTGCGTGCAGAACAGACACCAG
ACGAGTTAGAAATGGAAGATGGAGATGAGATCGATGCGATGTTGCATCAGACAGGC
GGTGCGGGTACCTGCGGTACCCAGGGCGGAAGCGGAGAAGAGGAAGAGAAGAAGA
AAGAAGAGGAAGAAAAGAAACAAAAAGAAGAACAAGAAAGACTTGCAAAAGAAG
AGGCAGAGAGAAAACAAAAAGAAGAACAAGAAAGACTTGCAAAAGAAGAGGCAG
AGAGAAAACAAAAGGAGGAAGAAGAGAGAAAACAAAAGGAAGAAGAAGAGAGAA
AACAAAAGGAGGAAGAAGAAAGAAAATTAAAGGAGGAACAAGAAAGAAAAGCTG
CAGAAGAAAAGAAAGCTAAAGAAGAAGCTGAGAGAAAGGCTAAAGAAGAACAAG
AAAGGAAAGCTGAAGAAGAGAGAAAGAAGAAAGAAGAGGAAGAAAGACTTGAAA
GAGAAAGAAAAGAGAGAGAAGAACAAGAAAAGAAAGCCAAAGAAGAGGCAGAGA
GAATTGCAAAGTTAGAGGCTGAAAAGAAGGCAGAAGAAGAAAGAAAAGCCAAAGA
AGAAGAAGAGAGAAAAGCCAAAGAAGAAGAGGAAAGAAAGAAGAAAGAGGAGCA
AGAAAGACTTGCAAAAGAAAAGGAAGAAGCAGAAAGAAAAGCTGCAGAGGAAAA
GAAAGCTAAAGAAGAACAAGAAAGAAAAGAAAAGGAAGAAGCAGAAAGAAAACT
TAAGGAAGAGGAAGAGAAGAAGAAAGAAGAGGAAGAAAAGAAACAAAAAGAAGA
ACAAGAAAGACTTGCAAAAGAAGAGGCAGAGAGAAAACAAAAAGAAGAACAAGA
AAGACTTGCAAAAGAAGAGGCAGAGAGAAAACAAAAGGAGGAAGAAGAGAGAAA
ACAAAAGGAAGAAGAAGAGAGAAAACAAAAGGAGGAAGAAGAAAGAAAATTAAA
GGAGGAACAAGAAAGAAAAGCTGCAGAAGAAAAGAAAGCTAAAGAAGAAGCTGA
GAGAAAGGCTAAAGAAGAACAAGAAAGGAAAGCTGAAGAAGAGAGAAAGAAGAA
AGAAGAGGAAGAAAGACTTGAAAGAGAAAGAAAAGAGAGAGAAGAACAAGAAAA
GAAAGCCAAAGAAGAGGCAGAGAGAATTGCAAAGTTAGAGGCTGAAAAGAAGGCA
GAAGAAGAAAGAAAAGCCAAAGAAGAAGAAGAGAGAAAAGCCAAAGAAGAAGAG
GAAAGAAAGAAGAAAGAGGAGCAAGAAAGACTTGCAAAAGAAAAGGAAGAAGCA
GAAAGAAAAGCTGCAGAGGAAAAGAAAGCTAAAGAAGAACAAGAAAGAAAAGAA
AAGGAAGAAGCAGAAAGAAAACAAAGAGGCTCTGGCGGCTCTGGCGGCGCCATGG
TAACCACCTTATCAGGTTTATCAGGTGAGCAAGGTCCGTCCGGTGATATGACAACTG
AAGAAGATAGTGCTACCCATATTAAATTCTCAAAACGTGATGAGGACGGCCGTGAG
TTAGCTGGTGCAACTATGGAGTTGCGTGATTCATCTGGTAAAACTATTAGTACATGG
ATTTCAGATGGACATGTGAAGGATTTCTACCTGTATCCAGGAAAATATACATTTGTC
GAAACCGCAGCACCAGACGGTTATGAGGTAGCAACTCCAATTGAATTTACAGTTAA
TGAGGACGGTCAGGTTACTGTAGATGGTGAAGCAACTGAAGGTGACGCTCATACTG
GATCCAGTGGTAGCTAGTAAGCTTAATTAGCTGAGCTTGGACTCCTGTTGATAGATC
CAGTAATGACCTCAGAACTCCATCTGGATTTGTTCAGAACGCTCGGTTGCCGCCGGG
CGTTTTTTATTGGTGAGAATCCAAGCTAGCCATGAAAATAAACTGTCTGCTTACATA
AACAGTAATACAAGGGGTGTTATGAGCCATATTCAACGGGAAACGTCTTGCTCTAG
GCCGCGATTAAATTCCAACATGGATGCTGATTTATATGGGTATAAATGGGCTCGCGA
TAATGTCGGGCAATCAGGTGCGACAATCTATCGATTGTATGGGAAGCCCGATGCGCC
AGAGTTGTTTCTGAAACATGGCAAAGGTAGCGTTGCCAATGATGTTACAGATGAGAT
GGTCAGACTAAACTGGCTGACGGAATTTATGCCTCTTCCGACCATCAAGCATTTTAT
CCGTACTCCTGATGATGCTTGGTTACTCACGACTGCGATCCCCGGCAAAACAGCATT
CCAGGTATTAGAAGAATATCCTGATTCAGGTGAAAATATTGTTGATGCGCTGGCAGT
GTTCCTGCGCCGGTTGCATTCGATTCCTGTTTGTAATTGTCCTTTTAACAGCGATCGC
GTATTTCGTCTCGCTCAGGCGCAATCACGAATGAATAACGGTTTGGTTGATGCGAGT
GATTTTGATGACGAGCGTAATGGCTGGCCTGTTGAACAAGTCTGGAAAGAAATGCA
CAAACTTTTGCCATTCTCACCGGATTCAGTCGTCACTCATGGTGATTTCTCACTTGAT
AACCTTATTTTTGACGAGGGGAAATTAATAGGTTGTATTGATGTTGGACGAGTCGGA
ATCGCAGACCGATACCAGGATCTTGCCATCCTATGGAACTGCCTCGGTGAGTTTTCT
CCTTCATTACAGAAACGGCTTTTTCAAAAATATGGTATTGATAATCCTGATATGAAT
AAATTGCAGTTTCATTTGATGCTCGATGAGTTTTTCTAAGAATTAATTCATGGGCAA
ATATTATACGCAAGGCGACAAGGTGCTGATGCCGCTGGCGATTCAGGTTCATCATGC
CGTTTGTGATGGCTTCCATGTCGGCAGAATGCTTAATGAATTACAACAGTACTGCGA
TGAGTGGCAGGGCGGGGCGTAATTTTTTTAAGGCAGTTATTGGTGCCCTTAAACGCC
TGGGGTAATGACTCTCTAGCTTGAGGCATCAAATAAAACGAAAGGCTCAGTCGAAA
GACTGGGCCTTTCGTTTTATCTGTTGTTTGTCGGTGAACGCTCTCCTGAGTAGGACAA
ATCCGCCCTCTAGATTACGTGCAGTCGATGATAAGCTGTCAAACATGAGAATTGTGC
CTAATGAGTGAGCTAACTTACATTAATTGCGTTGCGCTCACTGCCCGCTTTCCAGTCG
GGAAACCTGTCGTGCCAGCTGCATTAATGAATCGGCCAACGCGCGGGGAGAGGCGG
TTTGCGTATTGGGCGCCAGGGTGGTTTTTCTTTTCACCAGTGAGACGGGCAACAGCT
GATTGCCCTTCACCGCCTGGCCCTGAGAGAGTTGCAGCAAGCGGTCCACGCTGGTTT
GCCCCAGCAGGCGAAAATCCTGTTTGATGGTGGTTAACGGCGGGATATAACATGAG
CTGTCTTCGGTATCGTCGTATCCCACTACCGAGATATCCGCACCAACGCGCAGCCCG
GACTCGGTAATGGCGCGCATTGCGCCCAGCGCCATCTGATCGTTGGCAACCAGCATC
GCAGTGGGAACGATGCCCTCATTCAGCATTTGCATGGTTTGTTGAAAACCGGACATG
GCACTCCAGTCGCCTTCCCGTTCCGCTATCGGCTGAATTTGATTGCGAGTGAGATATT
TATGCCAGCCAGCCAGACGCAGACGCGCCGAGACAGAACTTAATGGGCCCGCTAAC
AGCGCGATTTGCTGGTGACCCAATGCGACCAGATGCTCCACGCCCAGTCGCGTACCG
TCTTCATGGGAGAAAATAATACTGTTGATGGGTGTCTGGTCAGAGACATCAAGAAAT
AACGCCGGAACATTAGTGCAGGCAGCTTCCACAGCAATGGCATCCTGGTCATCCAG
CGGATAGTTAATGATCAGCCCACTGACGCGTTGCGCGAGAAGATTGTGCACCGCCG
CTTTACAGGCTTCGACGCCGCTTCGTTCTACCATCGACACCACCACGCTGGCACCCA
GTTGATCGGCGCGAGATTTAATCGCCGCGACAATTTGCGACGGCGCGTGCAGGGCC
AGACTGGAGGTGGCAACGCCAATCAGCAACGACTGTTTGCCCGCCAGTTGTTGTGCC
ACGCGGTTGGGAATGTAATTCAGCTCCGCCATCGCCGCTTCCACTTTTTCCCGCGTTT
TCGCAGAAACGTGGCTGGCCTGGTTCACCACGCGGGAAACGGTCTGATAAGAGACA
CCGGCATACTCTGCGACATCGTATAACGTTACTGGTTTCACATTCACCACCCTGAATT
GACTCTCTTCCGGGCGCTATCATGCCATACCGCGAAAGGTTTTGCACCATTCGATGG
TGTCGGAATTTCGGGCAGCGTTGGGTCCTGGCCACGGGTGCGCATGATCTAGAGCTG
CCTCGCGCGTTTCGGTGATGACGGTGAAAACCTCTGACACATGCAGCTCCCGGAGAC
GGTCACAGCTTGTCTGTAAGCGGATGCCGGGAGCAGACAAGCCCGTCAGGGCGCGT
CAGCGGGTGTTGGCGGGTGTCGGGGCGCAGCCATGACCCAGTCACGTAGCGATAGC
GGAGTGTATACTGGCTTAACTATGCGGCATCAGAGCAGATTGTACTGAGAGTGCACC
ATATGCGGTGTGAAATACCGCACAGATGCGTAAGGAGAAAATACCGCATCAGGCGC
TCTTCCGCTTCCTCGCTCACTGACTCGCTGCGCTCGGTCGTTCGGCTGCGGCGAGCG
GTATCAGCTCACTCAAAGGCGGTAATACGGTTATCCACAGAATCAGGGGATAACGC
AGGAAAGAACATGTGAGCAAAAGGCCAGCAAAAGGCCAGGAACCGTAAAAAGGCC
GCGTTGCTGGCGTTTTTCCATAGGCTCCGCCCCCCTGACGAGCATCACAAAAATCGA
CGCTCAAGTCAGAGGTGGCGAAACCCGACAGGACTATAAAGATACCAGGCGTTTCC
CCCTGGAAGCTCCCTCGTGCGCTCTCCTGTTCCGACCCTGCCGCTTACCGGATACCTG
TCCGCCTTTCTCCCTTCGGGAAGCGTGGCGCTTTCTCATAGCTCACGCTGTAGGTATC
TCAGTTCGGTGTAGGTCGTTCGCTCCAAGCTGGGCTGTGTGCACGAACCCCCCGTTC
AGCCCGACCGCTGCGCCTTATCCGGTAACTATCGTCTTGAGTCCAACCCGGTAAGAC
ACGACTTATCGCCACTGGCAGCAGCCACTGGTAACAGGATTAGCAGAGCGAGGTAT
GTAGGCGGTGCTACAGAGTTCTTGAAGTGGTGGCCTAACTACGGCTACACTAGAAG
GACAGTATTTGGTATCTGCGCTCTGCTGAAGCCAGTTACCTTCGGAAAAAGAGTTGG
TAGCTCTTGATCCGGCAAACAAACCACCGCTGGTAGCGGTGGTTTTTTTGTTTGCAA
GCAGCAGATTACGCGCAGAAAAAAAGGATCTCAAGAAGATCCTTTGATCTTTTCTAC
GGGGTCTGACGCTCAGTGGAACGAAAACTCACG
>Biotin-60nm-SAH-SPYcatcher003
(SEQ ID NO: 6)
AGTC*GTQGGSGEEEEKKKEEEEKKQKEEQERLAKEEAERKQKEEQERLAKEEAERKQ
KEEEERKQKEEEERKQKEEEERKLKEEQERKAAEEKKAKEEAERKAKEEQERKAEEER
KKKEEEERLERERKEREEQEKKAKEEAERIAKLEAEKKAEEERKAKEEEERKAKEEEER
KKKEEQERLAKEKEEAERKAAEEKKAKEEQERKEKEEAERKLKEEEEKKKEEEEKKQK
EEQERLAKEEAERKQKEEQERLAKEEAERKQKEEEERKQKEEEERKQKEEEERKLKEEQ
ERKAAEEKKAKEEAERKAKEEQERKAEEERKKKEEEERLERERKEREEQEKKAKEEAE
RIAKLEAEKKAEEERKAKEEEERKAKEEEERKKKEEQERLAKEKEEAERKAAEEKKAK
EEQERKEKEEAERKQRGSGGSGGAMVTTLSGLSGEQGPSGDMTTEEDSATHIKESKRDE
DGRELAGATMELRDSSGKTISTWISDGHVKDFYLYPGKYTFVETAAPDGYEVATPIEFT
VNEDGQVTVDGEATEGDAHTGSSGS
[C* biotinylated cysteine]

Mono-SPYtag-avidin tetramer is purified by the previously described method with modifications (Howarth et al., 2006). pET21-SPY-His₆-tag streptavidin and pET21-streptavidin are generated by using pET21a-Streptavidin-Alive [Addgene plasmid #20860] (Howarth et al., 2006) as a PCR template. SPY-His₆-tag streptavidin and untagged avidin are expressed individually in E. coli BL21 (DE3) as inclusion bodies by 1 mM IPTG induction at 37° C. The protein-expressed cells are resuspended with 100 ml of buffer E (50 mM Tris-HCl, 1 mM EDTA) and disrupted by sonication. Insoluble fractions are collected by centrifugation at 20.000 rpm at 4° C. for 30 min using 45Ti rotor in Optima L80 (Beckman Coulter). Insoluble pellets are washed by resuspending them with 50 ml buffer E and re-collecting through centrifugation at 20,000 rpm at 4° C. for 30 min using 45Ti rotor in Optima L80 (Beckman Coulter). The washed insoluble pellets are resuspended by 8 ml of 6 M guanidine HCl (pH 1.5) and dialyzed against 200 ml of 6 M guanidine HCl (pH 1.5) overnight at 4° C. The denatured proteins are collected by centrifugation at 20,000 rpm at 4° C. for 30 min using 45Ti rotor in Optima L80 (Beckman Coulter). The protein concentrations in soluble fractions are estimated from 260 nm light adsorption. Denatured SPY-His₆-tag streptavidin and untagged streptavidin are mixed at the 1:2.9 molar ratio and rapidly refolded by diluting it with 250 ml PBS at 4° C. After 6 hours of stirring at 4° C., the aggregated proteins are removed with centrifugation at 20,000 rpm at 4° C. for 30 min using 45Ti rotor in Optima L80 (Beckman Coulter). The supernatant is mixed with 62.7 g of solid ammonium sulfate and stirred overnight at 4° C. The insolubilized proteins are removed with centrifugation at 20,000 rpm at 4° C. for 30 min using 45Ti rotor in Optima L80 (Beckman Coulter). The supernatant is loaded into the HisTrap HP column (Cytiva). Refolded avidin tetramers are eluted from the column by the linear gradient of imidazole (10 mM to 500 mM) in PBS. The peak corresponding mono-SPY-His-tagged streptavidin tetramer is collected and concentrated with Amicon 10K (Millipore). The concentrated mono-SPY-His₆-tagged streptavidin tetramer is purified through Hiload superdex75 (Cytiva) and stored at −20° C. in PBS containing 47.5% glycerol. Listed below are the DNA sequences of pET21-SPY-His₆-tag streptavidin and pET21-streptavidin and the amino acid sequence of SPY-His₆-tag streptavidin and streptavidin.

>pET21-SPY-His₆-tag streptavidin
(SEQ ID NO: 7)
TTCCCGTTCCGCTATCGGCTGAATTTGATTGCGAGTGAGATATTTATGCCAGCCAGC
CAGACGCAGACGCGCCGAGACAGAACTTAATGGGCCCGCTAACAGCGCGATTTGCT
GGTGACCCAATGCGACCAGATGCTCCACGCCCAGTCGCGTACCGTCTTCATGGGAG
AAAATAATACTGTTGATGGGTGTCTGGTCAGAGACATCAAGAAATAACGCCGGAAC
ATTAGTGCAGGCAGCTTCCACAGCAATGGCATCCTGGTCATCCAGCGGATAGTTAAT
GATCAGCCCACTGACGCGTTGCGCGAGAAGATTGTGCACCGCCGCTTTACAGGCTTC
GACGCCGCTTCGTTCTACCATCGACACCACCACGCTGGCACCCAGTTGATCGGCGCG
AGATTTAATCGCCGCGACAATTTGCGACGGCGCGTGCAGGGCCAGACTGGAGGTGG
CAACGCCAATCAGCAACGACTGTTTGCCCGCCAGTTGTTGTGCCACGCGGTTGGGAA
TGTAATTCAGCTCCGCCATCGCCGCTTCCACTTTTTCCCGCGTTTTCGCAGAAACGTG
GCTGGCCTGGTTCACCACGCGGGAAACGGTCTGATAAGAGACACCGGCATACTCTG
CGACATCGTATAACGTTACTGGTTTCACATTCACCACCCTGAATTGACTCTCTTCCGG
GCGCTATCATGCCATACCGCGAAAGGTTTTGCGCCATTCGATGGTGTCCGGGATCTC
GACGCTCTCCCTTATGCGACTCCTGCATTAGGAAGCAGCCCAGTAGTAGGTTGAGGC
CGTTGAGCACCGCCGCCGCAAGGAATGGTGCATGCAAGGAGATGGCGCCCAACAGT
CCCCCGGCCACGGGGCCTGCCACCATACCCACGCCGAAACAAGCGCTCATGAGCCC
GAAGTGGCGAGCCCGATCTTCCCCATCGGTGATGTCGGCGATATAGGCGCCAGCAA
CCGCACCTGTGGCGCCGGTGATGCCGGCCACGATGCGTCCGGCGTAGAGGATCGAG
ATCTCGATCCCGCGAAATTAATACGACTCACTATAGGGGAATTGTGAGCGGATAAC
AATTCCCCTCTAGAAATAATTTTGTTTAACTTTAAGAAGGAGATATACATATGCGTG
GTGTTCCGCACATCGTAATGGTTGACGCGTACAAACGTTACAAACATCACCATCACC
ACCATGGTGGTGGCGGTTCTGCTGAAGCTGGTATCACCGGCACCTGGTACAACCAGC
TGGGATCCACCTTCATCGTTACCGCTGGTGCTGACGGTGCTCTGACCGGTACCTACG
AATCCGCTGTTGGTAACGCTGAATCTAGATACGTTCTGACCGGTCGTTACGACTCCG
CTCCGGCTACCGACGGTTCCGGAACCGCTCTGGGTTGGACCGTTGCTTGGAAAAACA
ACTACCGTAACGCTCACTCCGCTACCACCTGGTCTGGCCAGTACGTTGGTGGTGCTG
AAGCTCGTATCAACACCCAGTGGTTGTTGACCTCCGGCACCACCGAAGCCAACGCGT
GGAAATCCACCCTGGTTGGTCACGACACCTTCACCAAAGTTAAACCGTCCGCTGCTT
CCTAATAAAAGCTTGCGGCCGCACTCGAGCACCACCACCACCACCACTGAGATCCG
GCTGCTAACAAAGCCCGAAAGGAAGCTGAGTTGGCTGCTGCCACCGCTGAGCAATA
ACTAGCATAACCCCTTGGGGCCTCTAAACGGGTCTTGAGGGGTTTTTTGCTGAAAGG
AGGAACTATATCCGGATTGGCGAATGGGACGCGCCCTGTAGCGGCGCATTAAGCGC
GGCGGGTGTGGTGGTTACGCGCAGCGTGACCGCTACACTTGCCAGCGCCCTAGCGC
CCGCTCCTTTCGCTTTCTTCCCTTCCTTTCTCGCCACGTTCGCCGGCTTTCCCCGTCAA
GCTCTAAATCGGGGGCTCCCTTTAGGGTTCCGATTTAGTGCTTTACGGCACCTCGAC
CCCAAAAAACTTGATTAGGGTGATGGTTCACGTAGTGGGCCATCGCCCTGATAGACG
GTTTTTCGCCCTTTGACGTTGGAGTCCACGTTCTTTAATAGTGGACTCTTGTTCCAAA
CTGGAACAACACTCAACCCTATCTCGGTCTATTCTTTTGATTTATAAGGGATTTTGCC
GATTTCGGCCTATTGGTTAAAAAATGAGCTGATTTAACAAAAATTTAACGCGAATTT
TAACAAAATATTAACGCTTACAATTTAGGTGGCACTTTTCGGGGAAATGTGCGCGGA
ACCCCTATTTGTTTATTTTTCTAAATACATTCAAATATGTATCCGCTCATGAGACAAT
AACCCTGATAAATGCTTCAATAATATTGAAAAAGGAAGAGTATGAGTATTCAACATT
TCCGTGTCGCCCTTATTCCCTTTTTTGCGGCATTTTGCCTTCCTGTTTTTGCTCACCCA
GAAACGCTGGTGAAAGTAAAAGATGCTGAAGATCAGTTGGGTGCACGAGTGGGTTA
CATCGAACTGGATCTCAACAGCGGTAAGATCCTTGAGAGTTTTCGCCCCGAAGAACG
TTTTCCAATGATGAGCACTTTTAAAGTTCTGCTATGTGGCGCGGTATTATCCCGTATT
GACGCCGGGCAAGAGCAACTCGGTCGCCGCATACACTATTCTCAGAATGACTTGGTT
GAGTACTCACCAGTCACAGAAAAGCATCTTACGGATGGCATGACAGTAAGAGAATT
ATGCAGTGCTGCCATAACCATGAGTGATAACACTGCGGCCAACTTACTTCTGACAAC
GATCGGAGGACCGAAGGAGCTAACCGCTTTTTTGCACAACATGGGGGATCATGTAA
CTCGCCTTGATCGTTGGGAACCGGAGCTGAATGAAGCCATACCAAACGACGAGCGT
GACACCACGATGCCTGCAGCAATGGCAACAACGTTGCGCAAACTATTAACTGGCGA
ACTACTTACTCTAGCTTCCCGGCAACAATTAATAGACTGGATGGAGGCGGATAAAGT
TGCAGGACCACTTCTGCGCTCGGCCCTTCCGGCTGGCTGGTTTATTGCTGATAAATCT
GGAGCCGGTGAGCGTGGGTCTCGCGGTATCATTGCAGCACTGGGGCCAGATGGTAA
GCCCTCCCGTATCGTAGTTATCTACACGACGGGGAGTCAGGCAACTATGGATGAACG
AAATAGACAGATCGCTGAGATAGGTGCCTCACTGATTAAGCATTGGTAACTGTCAG
ACCAAGTTTACTCATATATACTTTAGATTGATTTAAAACTTCATTTTTAATTTAAAAG
GATCTAGGTGAAGATCCTTTTTGATAATCTCATGACCAAAATCCCTTAACGTGAGTT
TTCGTTCCACTGAGCGTCAGACCCCGTAGAAAAGATCAAAGGATCTTCTTGAGATCC
TTTTTTTCTGCGCGTAATCTGCTGCTTGCAAACAAAAAAACCACCGCTACCAGCGGT
GGTTTGTTTGCCGGATCAAGAGCTACCAACTCTTTTTCCGAAGGTAACTGGCTTCAG
CAGAGCGCAGATACCAAATACTGTCCTTCTAGTGTAGCCGTAGTTAGGCCACCACTT
CAAGAACTCTGTAGCACCGCCTACATACCTCGCTCTGCTAATCCTGTTACCAGTGGC
TGCTGCCAGTGGCGATAAGTCGTGTCTTACCGGGTTGGACTCAAGACGATAGTTACC
GGATAAGGCGCAGCGGTCGGGCTGAACGGGGGGTTCGTGCACACAGCCCAGCTTGG
AGCGAACGACCTACACCGAACTGAGATACCTACAGCGTGAGCTATGAGAAAGCGCC
ACGCTTCCCGAAGGGAGAAAGGCGGACAGGTATCCGGTAAGCGGCAGGGTCGGAA
CAGGAGAGCGCACGAGGGAGCTTCCAGGGGGAAACGCCTGGTATCTTTATAGTCCT
GTCGGGTTTCGCCACCTCTGACTTGAGCGTCGATTTTTGTGATGCTCGTCAGGGGGG
CGGAGCCTATGGAAAAACGCCAGCAACGCGGCCTTTTTACGGTTCCTGGCCTTTTGC
TGGCCTTTTGCTCACATGTTCTTTCCTGCGTTATCCCCTGATTCTGTGGATAACCGTA
TTACCGCCTTTGAGTGAGCTGATACCGCTCGCCGCAGCCGAACGACCGAGCGCAGC
GAGTCAGTGAGCGAGGAAGCGGAAGAGCGCCTGATGCGGTATTTTCTCCTTACGCA
TCTGTGCGGTATTTCACACCGCAATGGTGCACTCTCAGTACAATCTGCTCTGATGCC
GCATAGTTAAGCCAGTATACACTCCGCTATCGCTACGTGACTGGGTCATGGCTGCGC
CCCGACACCCGCCAACACCCGCTGACGCGCCCTGACGGGCTTGTCTGCTCCCGGCAT
CCGCTTACAGACAAGCTGTGACCGTCTCCGGGAGCTGCATGTGTCAGAGGTTTTCAC
CGTCATCACCGAAACGCGCGAGGCAGCTGCGGTAAAGCTCATCAGCGTGGTCGTGA
AGCGATTCACAGATGTCTGCCTGTTCATCCGCGTCCAGCTCGTTGAGTTTCTCCAGA
AGCGTTAATGTCTGGCTTCTGATAAAGCGGGCCATGTTAAGGGCGGTTTTTTCCTGTT
TGGTCACTGATGCCTCCGTGTAAGGGGGATTTCTGTTCATGGGGGTAATGATACCGA
TGAAACGAGAGAGGATGCTCACGATACGGGTTACTGATGATGAACATGCCCGGTTA
CTGGAACGTTGTGAGGGTAAACAACTGGCGGTATGGATGCGGCGGGACCAGAGAAA
AATCACTCAGGGTCAATGCCAGCGCTTCGTTAATACAGATGTAGGTGTTCCACAGGG
TAGCCAGCAGCATCCTGCGATGCAGATCCGGAACATAATGGTGCAGGGCGCTGACT
TCCGCGTTTCCAGACTTTACGAAACACGGAAACCGAAGACCATTCATGTTGTTGCTC
AGGTCGCAGACGTTTTGCAGCAGCAGTCGCTTCACGTTCGCTCGCGTATCGGTGATT
CATTCTGCTAACCAGTAAGGCAACCCCGCCAGCCTAGCCGGGTCCTCAACGACAGG
AGCACGATCATGCGCACCCGTGGGGCCGCCATGCCGGCGATAATGGCCTGCTTCTCG
CCGAAACGTTTGGTGGCGGGACCAGTGACGAAGGCTTGAGCGAGGGCGTGCAAGAT
TCCGAATACCGCAAGCGACAGGCCGATCATCGTCGCGCTCCAGCGAAAGCGGTCCT
CGCCGAAAATGACCCAGAGCGCTGCCGGCACCTGTCCTACGAGTTGCATGATAAAG
AAGACAGTCATAAGTGCGGCGACGATAGTCATGCCCCGCGCCCACCGGAAGGAGCT
GACTGGGTTGAAGGCTCTCAAGGGCATCGGTCGAGATCCCGGTGCCTAATGAGTGA
GCTAACTTACATTAATTGCGTTGCGCTCACTGCCCGCTTTCCAGTCGGGAAACCTGTC
GTGCCAGCTGCATTAATGAATCGGCCAACGCGCGGGGAGAGGCGGTTTGCGTATTG
GGCGCCAGGGTGGTTTTTCTTTTCACCAGTGAGACGGGCAACAGCTGATTGCCCTTC
ACCGCCTGGCCCTGAGAGAGTTGCAGCAAGCGGTCCACGCTGGTTTGCCCCAGCAG
GCGAAAATCCTGTTTGATGGTGGTTAACGGCGGGATATAACATGAGCTGTCTTCGGT
ATCGTCGTATCCCACTACCGAGATATCCGCACCAACGCGCAGCCCGGACTCGGTAAT
GGCGCGCATTGCGCCCAGCGCCATCTGATCGTTGGCAACCAGCATCGCAGTGGGAA
CGATGCCCTCATTCAGCATTTGCATGGTTTGTTGAAAACCGGACATGGCACTCCAGT
CGCC
>pET21-streptavidin
(SEQ ID NO: 8)
TTCCCGTTCCGCTATCGGCTGAATTTGATTGCGAGTGAGATATTTATGCCAGCCAGC
CAGACGCAGACGCGCCGAGACAGAACTTAATGGGCCCGCTAACAGCGCGATTTGCT
GGTGACCCAATGCGACCAGATGCTCCACGCCCAGTCGCGTACCGTCTTCATGGGAG
AAAATAATACTGTTGATGGGTGTCTGGTCAGAGACATCAAGAAATAACGCCGGAAC
ATTAGTGCAGGCAGCTTCCACAGCAATGGCATCCTGGTCATCCAGCGGATAGTTAAT
GATCAGCCCACTGACGCGTTGCGCGAGAAGATTGTGCACCGCCGCTTTACAGGCTTC
GACGCCGCTTCGTTCTACCATCGACACCACCACGCTGGCACCCAGTTGATCGGCGCG
AGATTTAATCGCCGCGACAATTTGCGACGGCGCGTGCAGGGCCAGACTGGAGGTGG
CAACGCCAATCAGCAACGACTGTTTGCCCGCCAGTTGTTGTGCCACGCGGTTGGGAA
TGTAATTCAGCTCCGCCATCGCCGCTTCCACTTTTTCCCGCGTTTTCGCAGAAACGTG
GCTGGCCTGGTTCACCACGCGGGAAACGGTCTGATAAGAGACACCGGCATACTCTG
CGACATCGTATAACGTTACTGGTTTCACATTCACCACCCTGAATTGACTCTCTTCCGG
GCGCTATCATGCCATACCGCGAAAGGTTTTGCGCCATTCGATGGTGTCCGGGATCTC
GACGCTCTCCCTTATGCGACTCCTGCATTAGGAAGCAGCCCAGTAGTAGGTTGAGGC
CGTTGAGCACCGCCGCCGCAAGGAATGGTGCATGCAAGGAGATGGCGCCCAACAGT
CCCCCGGCCACGGGGCCTGCCACCATACCCACGCCGAAACAAGCGCTCATGAGCCC
GAAGTGGCGAGCCCGATCTTCCCCATCGGTGATGTCGGCGATATAGGCGCCAGCAA
CCGCACCTGTGGCGCCGGTGATGCCGGCCACGATGCGTCCGGCGTAGAGGATCGAG
ATCTCGATCCCGCGAAATTAATACGACTCACTATAGGGGAATTGTGAGCGGATAAC
AATTCCCCTCTAGAAATAATTTTGTTTAACTTTAAGAAGGAGATATACATATGGCTG
AAGCTGGTATCACCGGCACCTGGTACAACCAGCTGGGATCCACCTTCATCGTTACCG
CTGGTGCTGACGGTGCTCTGACCGGTACCTACGAATCCGCTGTTGGTAACGCTGAAT
CTAGATACGTTCTGACCGGTCGTTACGACTCCGCTCCGGCTACCGACGGTTCCGGAA
CCGCTCTGGGTTGGACCGTTGCTTGGAAAAACAACTACCGTAACGCTCACTCCGCTA
CCACCTGGTCTGGCCAGTACGTTGGTGGTGCTGAAGCTCGTATCAACACCCAGTGGT
TGTTGACCTCCGGCACCACCGAAGCCAACGCGTGGAAATCCACCCTGGTTGGTCACG
ACACCTTCACCAAAGTTAAACCGTCCGCTGCTTCCTAATAAAAGCTTGCGGCCGCAC
TCGAGCACCACCACCACCACCACTGAGATCCGGCTGCTAACAAAGCCCGAAAGGAA
GCTGAGTTGGCTGCTGCCACCGCTGAGCAATAACTAGCATAACCCCTTGGGGCCTCT
AAACGGGTCTTGAGGGGTTTTTTGCTGAAAGGAGGAACTATATCCGGATTGGCGAAT
GGGACGCGCCCTGTAGCGGCGCATTAAGCGCGGCGGGTGTGGTGGTTACGCGCAGC
GTGACCGCTACACTTGCCAGCGCCCTAGCGCCCGCTCCTTTCGCTTTCTTCCCTTCCT
TTCTCGCCACGTTCGCCGGCTTTCCCCGTCAAGCTCTAAATCGGGGGCTCCCTTTAGG
GTTCCGATTTAGTGCTTTACGGCACCTCGACCCCAAAAAACTTGATTAGGGTGATGG
TTCACGTAGTGGGCCATCGCCCTGATAGACGGTTTTTCGCCCTTTGACGTTGGAGTCC
ACGTTCTTTAATAGTGGACTCTTGTTCCAAACTGGAACAACACTCAACCCTATCTCG
GTCTATTCTTTTGATTTATAAGGGATTTTGCCGATTTCGGCCTATTGGTTAAAAAATG
AGCTGATTTAACAAAAATTTAACGCGAATTTTAACAAAATATTAACGCTTACAATTT
AGGTGGCACTTTTCGGGGAAATGTGCGCGGAACCCCTATTTGTTTATTTTTCTAAATA
CATTCAAATATGTATCCGCTCATGAGACAATAACCCTGATAAATGCTTCAATAATAT
TGAAAAAGGAAGAGTATGAGTATTCAACATTTCCGTGTCGCCCTTATTCCCTTTTTTG
CGGCATTTTGCCTTCCTGTTTTTGCTCACCCAGAAACGCTGGTGAAAGTAAAAGATG
CTGAAGATCAGTTGGGTGCACGAGTGGGTTACATCGAACTGGATCTCAACAGCGGT
AAGATCCTTGAGAGTTTTCGCCCCGAAGAACGTTTTCCAATGATGAGCACTTTTAAA
GTTCTGCTATGTGGCGCGGTATTATCCCGTATTGACGCCGGGCAAGAGCAACTCGGT
CGCCGCATACACTATTCTCAGAATGACTTGGTTGAGTACTCACCAGTCACAGAAAAG
CATCTTACGGATGGCATGACAGTAAGAGAATTATGCAGTGCTGCCATAACCATGAGT
GATAACACTGCGGCCAACTTACTTCTGACAACGATCGGAGGACCGAAGGAGCTAAC
CGCTTTTTTGCACAACATGGGGGATCATGTAACTCGCCTTGATCGTTGGGAACCGGA
GCTGAATGAAGCCATACCAAACGACGAGCGTGACACCACGATGCCTGCAGCAATGG
CAACAACGTTGCGCAAACTATTAACTGGCGAACTACTTACTCTAGCTTCCCGGCAAC
AATTAATAGACTGGATGGAGGCGGATAAAGTTGCAGGACCACTTCTGCGCTCGGCC
CTTCCGGCTGGCTGGTTTATTGCTGATAAATCTGGAGCCGGTGAGCGTGGGTCTCGC
GGTATCATTGCAGCACTGGGGCCAGATGGTAAGCCCTCCCGTATCGTAGTTATCTAC
ACGACGGGGAGTCAGGCAACTATGGATGAACGAAATAGACAGATCGCTGAGATAG
GTGCCTCACTGATTAAGCATTGGTAACTGTCAGACCAAGTTTACTCATATATACTTTA
GATTGATTTAAAACTTCATTTTTAATTTAAAAGGATCTAGGTGAAGATCCTTTTTGAT
AATCTCATGACCAAAATCCCTTAACGTGAGTTTTCGTTCCACTGAGCGTCAGACCCC
GTAGAAAAGATCAAAGGATCTTCTTGAGATCCTTTTTTTCTGCGCGTAATCTGCTGCT
TGCAAACAAAAAAACCACCGCTACCAGCGGTGGTTTGTTTGCCGGATCAAGAGCTA
CCAACTCTTTTTCCGAAGGTAACTGGCTTCAGCAGAGCGCAGATACCAAATACTGTC
CTTCTAGTGTAGCCGTAGTTAGGCCACCACTTCAAGAACTCTGTAGCACCGCCTACA
TACCTCGCTCTGCTAATCCTGTTACCAGTGGCTGCTGCCAGTGGCGATAAGTCGTGT
CTTACCGGGTTGGACTCAAGACGATAGTTACCGGATAAGGCGCAGCGGTCGGGCTG
AACGGGGGGTTCGTGCACACAGCCCAGCTTGGAGCGAACGACCTACACCGAACTGA
GATACCTACAGCGTGAGCTATGAGAAAGCGCCACGCTTCCCGAAGGGAGAAAGGCG
GACAGGTATCCGGTAAGCGGCAGGGTCGGAACAGGAGAGCGCACGAGGGAGCTTC
CAGGGGGAAACGCCTGGTATCTTTATAGTCCTGTCGGGTTTCGCCACCTCTGACTTG
AGCGTCGATTTTTGTGATGCTCGTCAGGGGGGCGGAGCCTATGGAAAAACGCCAGC
AACGCGGCCTTTTTACGGTTCCTGGCCTTTTGCTGGCCTTTTGCTCACATGTTCTTTCC
TGCGTTATCCCCTGATTCTGTGGATAACCGTATTACCGCCTTTGAGTGAGCTGATACC
GCTCGCCGCAGCCGAACGACCGAGCGCAGCGAGTCAGTGAGCGAGGAAGCGGAAG
AGCGCCTGATGCGGTATTTTCTCCTTACGCATCTGTGCGGTATTTCACACCGCAATGG
TGCACTCTCAGTACAATCTGCTCTGATGCCGCATAGTTAAGCCAGTATACACTCCGC
TATCGCTACGTGACTGGGTCATGGCTGCGCCCCGACACCCGCCAACACCCGCTGACG
CGCCCTGACGGGCTTGTCTGCTCCCGGCATCCGCTTACAGACAAGCTGTGACCGTCT
CCGGGAGCTGCATGTGTCAGAGGTTTTCACCGTCATCACCGAAACGCGCGAGGCAG
CTGCGGTAAAGCTCATCAGCGTGGTCGTGAAGCGATTCACAGATGTCTGCCTGTTCA
TCCGCGTCCAGCTCGTTGAGTTTCTCCAGAAGCGTTAATGTCTGGCTTCTGATAAAG
CGGGCCATGTTAAGGGCGGTTTTTTCCTGTTTGGTCACTGATGCCTCCGTGTAAGGG
GGATTTCTGTTCATGGGGGTAATGATACCGATGAAACGAGAGAGGATGCTCACGAT
ACGGGTTACTGATGATGAACATGCCCGGTTACTGGAACGTTGTGAGGGTAAACAAC
TGGCGGTATGGATGCGGCGGGACCAGAGAAAAATCACTCAGGGTCAATGCCAGCGC
TTCGTTAATACAGATGTAGGTGTTCCACAGGGTAGCCAGCAGCATCCTGCGATGCAG
ATCCGGAACATAATGGTGCAGGGCGCTGACTTCCGCGTTTCCAGACTTTACGAAACA
CGGAAACCGAAGACCATTCATGTTGTTGCTCAGGTCGCAGACGTTTTGCAGCAGCAG
TCGCTTCACGTTCGCTCGCGTATCGGTGATTCATTCTGCTAACCAGTAAGGCAACCC
CGCCAGCCTAGCCGGGTCCTCAACGACAGGAGCACGATCATGCGCACCCGTGGGGC
CGCCATGCCGGCGATAATGGCCTGCTTCTCGCCGAAACGTTTGGTGGCGGGACCAGT
GACGAAGGCTTGAGCGAGGGCGTGCAAGATTCCGAATACCGCAAGCGACAGGCCG
ATCATCGTCGCGCTCCAGCGAAAGCGGTCCTCGCCGAAAATGACCCAGAGCGCTGC
CGGCACCTGTCCTACGAGTTGCATGATAAAGAAGACAGTCATAAGTGCGGCGACGA
TAGTCATGCCCCGCGCCCACCGGAAGGAGCTGACTGGGTTGAAGGCTCTCAAGGGC
ATCGGTCGAGATCCCGGTGCCTAATGAGTGAGCTAACTTACATTAATTGCGTTGCGC
TCACTGCCCGCTTTCCAGTCGGGAAACCTGTCGTGCCAGCTGCATTAATGAATCGGC
CAACGCGCGGGGAGAGGCGGTTTGCGTATTGGGCGCCAGGGTGGTTTTTCTTTTCAC
CAGTGAGACGGGCAACAGCTGATTGCCCTTCACCGCCTGGCCCTGAGAGAGTTGCA
GCAAGCGGTCCACGCTGGTTTGCCCCAGCAGGCGAAAATCCTGTTTGATGGTGGTTA
ACGGCGGGATATAACATGAGCTGTCTTCGGTATCGTCGTATCCCACTACCGAGATAT
CCGCACCAACGCGCAGCCCGGACTCGGTAATGGCGCGCATTGCGCCCAGCGCCATC
TGATCGTTGGCAACCAGCATCGCAGTGGGAACGATGCCCTCATTCAGCATTTGCATG
GTTTGTTGAAAACCGGACATGGCACTCCAGTCGCC
>SPY-His₆-tag streptavidin
(SEQ ID NO: 9)
MRGVPHIVMVDAYKRYKHHHHHHGGGGSAEAGITGTWYNQLGSTFIVTAGADGALTG
TYESAVGNAESRYVLTGRYDSAPATDGSGTALGWTVAWKNNYRNAHSATTWSGQYV
GGAEARINTQWLLTSGTTEANAWKSTLVGHDTFTKVKPSAAS
>Streptavidin
(SEQ ID NO: 10)
MAEAGITGTWYNQLGSTFIVTAGADGALTGTYESAVGNAESRYVLTGRYDSAPATDGS
GTALGWTVAWKNNYRNAHSATTWSGQYVGGAEARINTQWLLTSGTTEANAWKSTLV
GHDTFTKVKPSAAS

His₆-SPYtag-tandem GFP nanobody is purified by the previously described method with modifications (Zhang et al., 2020). pET28b-His-SPY-Tandem GFP nanobody is built by inserting SPY-tag coded sequence into pN8his-GFPenhancer-GGGGS4-LaG16 [Addgene plasmid #140442] (Zhang et al., 2020). His₆-SPYtag-tandem-GFP-nanobody is expressed at 10° C. in E. coli Rosetta (DE3) by IPTG induction. The cells expressing His₆-SPYtag-tandem-GFP-nanobody are resuspended with 100 ml buffer A and are disrupted by sonication. The soluble fraction is collected with centrifugation at 20,000 rpm (46,502 rcf) at 4° C. for 30 min using 45Ti rotor in Optima L80 (Beckman Coulter) and applied to the HisTrap HP column (Cytiva). The protein is eluted from column with the step gradient of imidazole (50, 200, 400 mM) in buffer F (50 mM Tris-HCl (pH 8), 100 mM NaCl, 800 mM Imidazole, 5% Glycerol). The eluted protein is concentrated with Amicon 10K (Millipore). The concentrated His₆-SPYtag-tandem GFP nanobody is further purified through Hiload superdex75 (Cytiva) and stored at −20° C. in PBS containing 47.5% glycerol. Listed below are the DNA sequence of pET28b-His-SPY-Tandem GFP nanobody and the amino acid sequence of His₆-SPYtag-tandem-GFP-nanobody.

>pET28b-His-SPY-Tandem GFP nanobody
(SEQ ID NO: 11)
TAATACGACTCACTATAGGGGAATTGTGAGCGGATAACAATTCCCCTCTAGAAATAA
TTTTGTTTAACTTTAAGAAGGAGATATACCATGGGCAGCAGCCATCATCATCATCAT
CATCATCACAGCAGCGGCCGTGGTGTCCCGCACATCGTTATGGTTGACGCGTACAAA
CGTTACAAACATATGGCCCAAGTTCAGCTGGTTGAGAGTGGTGGTGCGCTGGTTCAG
CCGGGTGGTAGTCTGCGTCTCAGTTGCGCCGCGAGCGGTTTCCCGGTGAATCGCTAC
AGTATGCGCTGGTACCGTCAAGCCCCGGGCAAAGAACGCGAATGGGTTGCCGGTAT
GAGTAGTGCCGGTGATCGCAGCAGCTATGAAGACAGCGTGAAAGGCCGCTTTACCA
TCAGCCGCGATGATGCGCGCAATACCGTGTACCTCCAGATGAACAGTCTGAAGCCA
GAGGATACCGCCGTGTACTACTGCAACGTGAACGTGGGCTTCGAATATTGGGGTCA
AGGCACGCAAGTTACCGTTAGTGGCGGCGGCGGTAGCGGCGGTGGCGGTAGTGGTG
GCGGCGGCAGCGGTGGCGGTGGTAGCGCCCAAGTTCAGCTGGTTGAAAGCGGCGGT
CGTCTGGTTCAAGCCGGTGATAGTCTGCGCCTCAGTTGTGCGGCCAGTGGTCGCACC
TTCAGCACCAGCGCCATGGCGTGGTTTCGTCAAGCCCCGGGCCGCGAACGCGAATTT
GTTGCCGCCATCACGTGGACGGTTGGCAACACCATCCTCGGCGATAGCGTTAAAGG
CCGCTTCACCATTAGCCGCGATCGCGCGAAAAACACCGTGGATCTCCAGATGGACA
ACCTCGAACCGGAAGATACCGCGGTGTACTATTGCAGCGCCCGTAGCCGCGGCTAT
GTGCTGAGCGTGCTGCGCAGCGTTGATAGCTACGACTACTGGGGCCAAGGCACCCA
AGTTACCGTGAGCTAAGGATCCGAATTCGAGCTCCGTCGACAAGCTTGCGGCCGCA
CTCGAGCACCACCACCACCACCACTGAGATCCGGCTGCTAACAAAGCCCGAAAGGA
AGCTGAGTTGGCTGCTGCCACCGCTGAGCAATAACTAGCATAACCCCTTGGGGCCTC
TAAACGGGTCTTGAGGGGTTTTTTGCTGAAAGGAGGAACTATATCCGGATTGGCGAA
TGGGACGCGCCCTGTAGCGGCGCATTAAGCGCGGCGGGTGTGGTGGTTACGCGCAG
CGTGACCGCTACACTTGCCAGCGCCCTAGCGCCCGCTCCTTTCGCTTTCTTCCCTTCC
TTTCTCGCCACGTTCGCCGGCTTTCCCCGTCAAGCTCTAAATCGGGGGCTCCCTTTAG
GGTTCCGATTTAGTGCTTTACGGCACCTCGACCCCAAAAAACTTGATTAGGGTGATG
GTTCACGTAGTGGGCCATCGCCCTGATAGACGGTTTTTCGCCCTTTGACGTTGGAGT
CCACGTTCTTTAATAGTGGACTCTTGTTCCAAACTGGAACAACACTCAACCCTATCTC
GGTCTATTCTTTTGATTTATAAGGGATTTTGCCGATTTCGGCCTATTGGTTAAAAAAT
GAGCTGATTTAACAAAAATTTAACGCGAATTTTAACAAAATATTAACGCTTACAATT
TAGGTGGCACTTTTCGGGGAAATGTGCGCGGAACCCCTATTTGTTTATTTTTCTAAAT
ACATTCAAATATGTATCCGCTCATGAATTAATTCTTAGAAAAACTCATCGAGCATCA
AATGAAACTGCAATTTATTCATATCAGGATTATCAATACCATATTTTTGAAAAAGCC
GTTTCTGTAATGAAGGAGAAAACTCACCGAGGCAGTTCCATAGGATGGCAAGATCC
TGGTATCGGTCTGCGATTCCGACTCGTCCAACATCAATACAACCTATTAATTTCCCCT
CGTCAAAAATAAGGTTATCAAGTGAGAAATCACCATGAGTGACGACTGAATCCGGT
GAGAATGGCAAAAGTTTATGCATTTCTTTCCAGACTTGTTCAACAGGCCAGCCATTA
CGCTCGTCATCAAAATCACTCGCATCAACCAAACCGTTATTCATTCGTGATTGCGCC
TGAGCGAGACGAAATACGCGATCGCTGTTAAAAGGACAATTACAAACAGGAATCGA
ATGCAACCGGCGCAGGAACACTGCCAGCGCATCAACAATATTTTCACCTGAATCAG
GATATTCTTCTAATACCTGGAATGCTGTTTTCCCGGGGATCGCAGTGGTGAGTAACC
ATGCATCATCAGGAGTACGGATAAAATGCTTGATGGTCGGAAGAGGCATAAATTCC
GTCAGCCAGTTTAGTCTGACCATCTCATCTGTAACATCATTGGCAACGCTACCTTTGC
CATGTTTCAGAAACAACTCTGGCGCATCGGGCTTCCCATACAATCGATAGATTGTCG
CACCTGATTGCCCGACATTATCGCGAGCCCATTTATACCCATATAAATCAGCATCCA
TGTTGGAATTTAATCGCGGCCTAGAGCAAGACGTTTCCCGTTGAATATGGCTCATAA
CACCCCTTGTATTACTGTTTATGTAAGCAGACAGTTTTATTGTTCATGACCAAAATCC
CTTAACGTGAGTTTTCGTTCCACTGAGCGTCAGACCCCGTAGAAAAGATCAAAGGAT
CTTCTTGAGATCCTTTTTTTCTGCGCGTAATCTGCTGCTTGCAAACAAAAAAACCACC
GCTACCAGCGGTGGTTTGTTTGCCGGATCAAGAGCTACCAACTCTTTTTCCGAAGGT
AACTGGCTTCAGCAGAGCGCAGATACCAAATACTGTCCTTCTAGTGTAGCCGTAGTT
AGGCCACCACTTCAAGAACTCTGTAGCACCGCCTACATACCTCGCTCTGCTAATCCT
GTTACCAGTGGCTGCTGCCAGTGGCGATAAGTCGTGTCTTACCGGGTTGGACTCAAG
ACGATAGTTACCGGATAAGGCGCAGCGGTCGGGCTGAACGGGGGGTTCGTGCACAC
AGCCCAGCTTGGAGCGAACGACCTACACCGAACTGAGATACCTACAGCGTGAGCTA
TGAGAAAGCGCCACGCTTCCCGAAGGGAGAAAGGCGGACAGGTATCCGGTAAGCG
GCAGGGTCGGAACAGGAGAGCGCACGAGGGAGCTTCCAGGGGGAAACGCCTGGTA
TCTTTATAGTCCTGTCGGGTTTCGCCACCTCTGACTTGAGCGTCGATTTTTGTGATGC
TCGTCAGGGGGGCGGAGCCTATGGAAAAACGCCAGCAACGCGGCCTTTTTACGGTT
CCTGGCCTTTTGCTGGCCTTTTGCTCACATGTTCTTTCCTGCGTTATCCCCTGATTCTG
TGGATAACCGTATTACCGCCTTTGAGTGAGCTGATACCGCTCGCCGCAGCCGAACGA
CCGAGCGCAGCGAGTCAGTGAGCGAGGAAGCGGAAGAGCGCCTGATGCGGTATTTT
CTCCTTACGCATCTGTGCGGTATTTCACACCGCAATGGTGCACTCTCAGTACAATCTG
CTCTGATGCCGCATAGTTAAGCCAGTATACACTCCGCTATCGCTACGTGACTGGGTC
ATGGCTGCGCCCCGACACCCGCCAACACCCGCTGACGCGCCCTGACGGGCTTGTCTG
CTCCCGGCATCCGCTTACAGACAAGCTGTGACCGTCTCCGGGAGCTGCATGTGTCAG
AGGTTTTCACCGTCATCACCGAAACGCGCGAGGCAGCTGCGGTAAAGCTCATCAGC
GTGGTCGTGAAGCGATTCACAGATGTCTGCCTGTTCATCCGCGTCCAGCTCGTTGAG
TTTCTCCAGAAGCGTTAATGTCTGGCTTCTGATAAAGCGGGCCATGTTAAGGGCGGT
TTTTTCCTGTTTGGTCACTGATGCCTCCGTGTAAGGGGGATTTCTGTTCATGGGGGTA
ATGATACCGATGAAACGAGAGAGGATGCTCACGATACGGGTTACTGATGATGAACA
TGCCCGGTTACTGGAACGTTGTGAGGGTAAACAACTGGCGGTATGGATGCGGCGGG
ACCAGAGAAAAATCACTCAGGGTCAATGCCAGCGCTTCGTTAATACAGATGTAGGT
GTTCCACAGGGTAGCCAGCAGCATCCTGCGATGCAGATCCGGAACATAATGGTGCA
GGGCGCTGACTTCCGCGTTTCCAGACTTTACGAAACACGGAAACCGAAGACCATTC
ATGTTGTTGCTCAGGTCGCAGACGTTTTGCAGCAGCAGTCGCTTCACGTTCGCTCGC
GTATCGGTGATTCATTCTGCTAACCAGTAAGGCAACCCCGCCAGCCTAGCCGGGTCC
TCAACGACAGGAGCACGATCATGCGCACCCGTGGGGCCGCCATGCCGGCGATAATG
GCCTGCTTCTCGCCGAAACGTTTGGTGGCGGGACCAGTGACGAAGGCTTGAGCGAG
GGCGTGCAAGATTCCGAATACCGCAAGCGACAGGCCGATCATCGTCGCGCTCCAGC
GAAAGCGGTCCTCGCCGAAAATGACCCAGAGCGCTGCCGGCACCTGTCCTACGAGT
TGCATGATAAAGAAGACAGTCATAAGTGCGGCGACGATAGTCATGCCCCGCGCCCA
CCGGAAGGAGCTGACTGGGTTGAAGGCTCTCAAGGGCATCGGTCGAGATCCCGGTG
CCTAATGAGTGAGCTAACTTACATTAATTGCGTTGCGCTCACTGCCCGCTTTCCAGTC
GGGAAACCTGTCGTGCCAGCTGCATTAATGAATCGGCCAACGCGCGGGGAGAGGCG
GTTTGCGTATTGGGCGCCAGGGTGGTTTTTCTTTTCACCAGTGAGACGGGCAACAGC
TGATTGCCCTTCACCGCCTGGCCCTGAGAGAGTTGCAGCAAGCGGTCCACGCTGGTT
TGCCCCAGCAGGCGAAAATCCTGTTTGATGGTGGTTAACGGCGGGATATAACATGA
GCTGTCTTCGGTATCGTCGTATCCCACTACCGAGATATCCGCACCAACGCGCAGCCC
GGACTCGGTAATGGCGCGCATTGCGCCCAGCGCCATCTGATCGTTGGCAACCAGCAT
CGCAGTGGGAACGATGCCCTCATTCAGCATTTGCATGGTTTGTTGAAAACCGGACAT
GGCACTCCAGTCGCCTTCCCGTTCCGCTATCGGCTGAATTTGATTGCGAGTGAGATA
TTTATGCCAGCCAGCCAGACGCAGACGCGCCGAGACAGAACTTAATGGGCCCGCTA
ACAGCGCGATTTGCTGGTGACCCAATGCGACCAGATGCTCCACGCCCAGTCGCGTAC
CGTCTTCATGGGAGAAAATAATACTGTTGATGGGTGTCTGGTCAGAGACATCAAGA
AATAACGCCGGAACATTAGTGCAGGCAGCTTCCACAGCAATGGCATCCTGGTCATC
CAGCGGATAGTTAATGATCAGCCCACTGACGCGTTGCGCGAGAAGATTGTGCACCG
CCGCTTTACAGGCTTCGACGCCGCTTCGTTCTACCATCGACACCACCACGCTGGCAC
CCAGTTGATCGGCGCGAGATTTAATCGCCGCGACAATTTGCGACGGCGCGTGCAGG
GCCAGACTGGAGGTGGCAACGCCAATCAGCAACGACTGTTTGCCCGCCAGTTGTTGT
GCCACGCGGTTGGGAATGTAATTCAGCTCCGCCATCGCCGCTTCCACTTTTTCCCGC
GTTTTCGCAGAAACGTGGCTGGCCTGGTTCACCACGCGGGAAACGGTCTGATAAGA
GACACCGGCATACTCTGCGACATCGTATAACGTTACTGGTTTCACATTCACCACCCT
GAATTGACTCTCTTCCGGGCGCTATCATGCCATACCGCGAAAGGTTTTGCGCCATTC
GATGGTGTCCGGGATCTCGACGCTCTCCCTTATGCGACTCCTGCATTAGGAAGCAGC
CCAGTAGTAGGTTGAGGCCGTTGAGCACCGCCGCCGCAAGGAATGGTGCATGCAAG
GAGATGGCGCCCAACAGTCCCCCGGCCACGGGGCCTGCCACCATACCCACGCCGAA
ACAAGCGCTCATGAGCCCGAAGTGGCGAGCCCGATCTTCCCCATCGGTGATGTCGG
CGATATAGGCGCCAGCAACCGCACCTGTGGCGCCGGTGATGCCGGCCACGATGCGT
CCGGCGTAGAGGATCGAGATCTCGATCCCGCGAAAT
>His6-SPYtag-tandem GFP nanobody
(SEQ ID NO: 12)
MGSSHHHHHHHHSSGRGVPHIVMVDAYKRYKHMAQVQLVESGGALVQPGGSLRLSC
AASGFPVNRYSMRWYRQAPGKEREWVAGMSSAGDRSSYEDSVKGRFTISRDDARNTV
YLQMNSLKPEDTAVYYCNVNVGFEYWGQGTQVTVSGGGGSGGGGSGGGGSGGGGSA
QVQLVESGGRLVQAGDSLRLSCAASGRTESTSAMAWFRQAPGREREFVAAITWTVGNT
ILGDSVKGRFTISRDRAKNTVDLQMDNLEPEDTAVYYCSARSRGYVLSVLRSVDSYDY
WGQGTQVTVS

To purify Xenopus laevis H1.8-GFP, pQE80-His 14-bdSUMO-GFP-H1.8 that encoded the SENP1 protease site at the C-terminus of bdSUMO is generated. Using this plasmid, His₁₄-bdSUMO-GFP-H1.8 is expressed in E. coli Rosetta (DE3) at 18° C. with 1 mM IPTG induction. The soluble fraction is collected with centrifugation at 20,000 rpm (46,502 rcf) at 4° C. for 30 min using 45Ti rotor in Optima L80 (Beckman Coulter) and applied to the HisTrap HP column (Cytiva). His₁₄-bdSUMO-GFP-H1.8 are eluted from the column with the linear gradient of imidazole (100 mM to 800 mM) in PBS. The His₁₄-bdSUMO-GFP-H1.8 containing fractions are collected, mixed with SENP1 protease, and dialyzed overnight against PBS containing 5% glycerol at 4° C. The SENP1 treated sample is applied for Heparin HP column (Cytiva) and eluted with a linear gradient of NaCl (137 mM to 937 mM) in PBS containing 5% glycerol. The GFP-H1.8 containing fractions are collected and concentrated using Amicon 30K (Millipore) and applied to Hiload Superdex200 16/600 (Cytiva) in PBS containing 5% glycerol. The GFP-H1.8 containing fractions are collected, concentrated using Amicon 30K (Millipore), flash frozen, and stored at −80° C. Listed below are the DNA sequence of pQE80-His₁₄-bdSUMO-GFP-H1.8 and the amino acid sequence of GFP-H1.8.

>pQE80-His14-bdSUMO-GFP-H1.8
(SEQ ID NO: 13)
TTAAGGGATTTTGGTCATGACATTAACCTATAAAAATAGGCGTATCACGAGGCCCTT
TCGTCTTCACCTCGAGAAATCATAAAAAATTTATTTGCTTTGTGAGCGGATAACAAT
TATAATAGATTCAATTGTGAGCGGATAACAATTTCACACAGAATTCATTAAAGAGGA
GAAATTAACCATGAATCACAAAGTGAGCAAGCATCACCATCATTCAGGCCATCACC
ATACCGGACACCACCATCATTCAGGCAGTCATCACCATACCGGCGAGAACCTGTATT
TTCAGGGTTCAGCTGCAGGCGGTGAAGAGGATAAAAAGCCAGCCGGAGGGGAAGG
TGGTGGTGCTCATATTAACTTAAAGGTTAAAGGACAGGATGGCAACGAGGTGTTCTT
CCGCATTAAGCGCAGTACGCAACTGAAAAAACTGATGAACGCCTACTGTGACCGTC
AATCAGTTGACATGACTGCCATTGCGTTCTTATTTGACGGTCGCCGCCTGCGTGCAG
AACAGACACCAGACGAGTTAGAAATGGAAGATGGAGATGAGATCGATGCGATGTTG
CATCAGACAGGCGGTATGGCTCCTAAGAAGGCAGTTGCTGCACCTGAGGGAGGCAA
CAAGGAAAATGCAGCAGTAAAAGGATCCAGTAAAGTTAAGGTTAAAAGAAAATCTA
TCAAACTAGTCAAGACCCAATCACATCCCCCAACCCTGTCGATGGTGGTGGAGGTCC
TGAAAAAGAACACGGAGCGGAAAGGGACCTCTGTGCAGGCCATTCGGACCCGGATT
CTGTCTGCACATCCCACAGTGGATCCACTGAGGCTGAAGTTTTTGCTACGGACGGCC
CTGAACAAAGGGCTAGAGAAGGGGATTCTGATCAGACCTCTAAACTCTAGTGCAAC
AGGAGCTACAGGAAGATTCAAACTTGCCAAACCAGTAAAAACTACAAAGGCTGGGA
AAGAAAATGTAGCGTCTGAAAACGTAGACCCAAATGCAGAGCAGGAAACCCAAAA
GAAGGCCCCAAAGAAAGAAAAGAAAGCGAAGACTGAGAAAGAACCCAAAGGTGAG
AAAACCAAAGCTGTAGCTAAAAAGGCCAAGGAAGATTCTGATGAAAAACCCAAAGT
TGCCAAATCTAAGAAAGATAAAGAGGCAAAAGAAGTTGACAAGGCTAATAAAGAG
GCAAAAGAAGTTGACAAGGCTAATAAAGAGGCAAAAGAAGTTGACAAGGCTCCGG
CAAAGAAACCAAAAGCCAAAACAGAGGCTGCGAAAGCTGAGGGGGGTGGCAAGGC
AAAGAAGGAGCCCCCAAAGGCCAAAGCCAAGGACGTGAAAGCACAGAAGGACTCT
ACAGATGAAGGTGCTCCAGTTAAGGCTGGCAAGAAAGGAAAGAAAGTGACAAACG
GTGGTGGCGGTTCTGGCGGCGGTGGCAGCATGAGCAAAGGGGAAGAACTGTTTACC
GGCGTGGTGCCGATTCTGGTGGAGCTGGATGGTGATGTGAATGGGCATAAGTTTAGC
GTGCGTGGCGAAGGCGAGGGCGACGCGACCAATGGCAAGCTGACCCTGAAGTTCAT
TTGCACCACCGGCAAACTGCCCGTGCCGTGGCCGACCCTGGTGACCACCCTGACCTA
TGGCGTGCAGTGCTTTAGCCGTTATCCAGATCACATGAAACGTCATGATTTCTTTAA
GAGCGCGATGCCGGAAGGCTATGTGCAGGAACGTACCATTAGCTTTAAGGATGATG
GCACCTATAAAACCCGTGCGGAAGTGAAATTTGAGGGGGACACCCTTGTAAATCGT
ATTGAACTGAAAGGCATTGACTTCAAGGAAGACGGCAATATTCTGGGCCATAAACT
GGAATACAACTTTAATAGCCATAACGTGTATATTACCGCGGATAAACAGAAAAATG
GCATTAAAGCGAATTTCAAAATCCGTCATAATGTGGAAGATGGCAGCGTTCAGCTG
GCGGATCACTATCAGCAGAATACCCCGATTGGCGATGGCCCGGTGTTGCTGCCGGAT
AATCATTACCTGAGCACCCAGAGCGTGCTGAGCAAGGATCCGAATGAAAAACGTGA
TCATATGGTGCTGTTGGAATTTGTGACCGCAGCGGGGATTACCCATGGCATGGATGA
ACTGTATAAATAGTAAGCTTAATTAGCTGAGCTTGGACTCCTGTTGATAGATCCAGT
AATGACCTCAGAACTCCATCTGGATTTGTTCAGAACGCTCGGTTGCCGCCGGGCGTT
TTTTATTGGTGAGAATCCAAGCTAGCCATGAAAATAAACTGTCTGCTTACATAAACA
GTAATACAAGGGGTGTTATGAGCCATATTCAACGGGAAACGTCTTGCTCTAGGCCGC
GATTAAATTCCAACATGGATGCTGATTTATATGGGTATAAATGGGCTCGCGATAATG
TCGGGCAATCAGGTGCGACAATCTATCGATTGTATGGGAAGCCCGATGCGCCAGAG
TTGTTTCTGAAACATGGCAAAGGTAGCGTTGCCAATGATGTTACAGATGAGATGGTC
AGACTAAACTGGCTGACGGAATTTATGCCTCTTCCGACCATCAAGCATTTTATCCGT
ACTCCTGATGATGCTTGGTTACTCACGACTGCGATCCCCGGCAAAACAGCATTCCAG
GTATTAGAAGAATATCCTGATTCAGGTGAAAATATTGTTGATGCGCTGGCAGTGTTC
CTGCGCCGGTTGCATTCGATTCCTGTTTGTAATTGTCCTTTTAACAGCGATCGCGTAT
TTCGTCTCGCTCAGGCGCAATCACGAATGAATAACGGTTTGGTTGATGCGAGTGATT
TTGATGACGAGCGTAATGGCTGGCCTGTTGAACAAGTCTGGAAAGAAATGCACAAA
CTTTTGCCATTCTCACCGGATTCAGTCGTCACTCATGGTGATTTCTCACTTGATAACC
TTATTTTTGACGAGGGGAAATTAATAGGTTGTATTGATGTTGGACGAGTCGGAATCG
CAGACCGATACCAGGATCTTGCCATCCTATGGAACTGCCTCGGTGAGTTTTCTCCTTC
ATTACAGAAACGGCTTTTTCAAAAATATGGTATTGATAATCCTGATATGAATAAATT
GCAGTTTCATTTGATGCTCGATGAGTTTTTCTAAGAATTAATTCATGGGCAAATATTA
TACGCAAGGCGACAAGGTGCTGATGCCGCTGGCGATTCAGGTTCATCATGCCGTTTG
TGATGGCTTCCATGTCGGCAGAATGCTTAATGAATTACAACAGTACTGCGATGAGTG
GCAGGGCGGGGCGTAATTTTTTTAAGGCAGTTATTGGTGCCCTTAAACGCCTGGGGT
AATGACTCTCTAGCTTGAGGCATCAAATAAAACGAAAGGCTCAGTCGAAAGACTGG
GCCTTTCGTTTTATCTGTTGTTTGTCGGTGAACGCTCTCCTGAGTAGGACAAATCCGC
CCTCTAGATTACGTGCAGTCGATGATAAGCTGTCAAACATGAGAATTGTGCCTAATG
AGTGAGCTAACTTACATTAATTGCGTTGCGCTCACTGCCCGCTTTCCAGTCGGGAAA
CCTGTCGTGCCAGCTGCATTAATGAATCGGCCAACGCGCGGGGAGAGGCGGTTTGC
GTATTGGGCGCCAGGGTGGTTTTTCTTTTCACCAGTGAGACGGGCAACAGCTGATTG
CCCTTCACCGCCTGGCCCTGAGAGAGTTGCAGCAAGCGGTCCACGCTGGTTTGCCCC
AGCAGGCGAAAATCCTGTTTGATGGTGGTTAACGGCGGGATATAACATGAGCTGTCT
TCGGTATCGTCGTATCCCACTACCGAGATATCCGCACCAACGCGCAGCCCGGACTCG
GTAATGGCGCGCATTGCGCCCAGCGCCATCTGATCGTTGGCAACCAGCATCGCAGTG
GGAACGATGCCCTCATTCAGCATTTGCATGGTTTGTTGAAAACCGGACATGGCACTC
CAGTCGCCTTCCCGTTCCGCTATCGGCTGAATTTGATTGCGAGTGAGATATTTATGCC
AGCCAGCCAGACGCAGACGCGCCGAGACAGAACTTAATGGGCCCGCTAACAGCGCG
ATTTGCTGGTGACCCAATGCGACCAGATGCTCCACGCCCAGTCGCGTACCGTCTTCA
TGGGAGAAAATAATACTGTTGATGGGTGTCTGGTCAGAGACATCAAGAAATAACGC
CGGAACATTAGTGCAGGCAGCTTCCACAGCAATGGCATCCTGGTCATCCAGCGGAT
AGTTAATGATCAGCCCACTGACGCGTTGCGCGAGAAGATTGTGCACCGCCGCTTTAC
AGGCTTCGACGCCGCTTCGTTCTACCATCGACACCACCACGCTGGCACCCAGTTGAT
CGGCGCGAGATTTAATCGCCGCGACAATTTGCGACGGCGCGTGCAGGGCCAGACTG
GAGGTGGCAACGCCAATCAGCAACGACTGTTTGCCCGCCAGTTGTTGTGCCACGCG
GTTGGGAATGTAATTCAGCTCCGCCATCGCCGCTTCCACTTTTTCCCGCGTTTTCGCA
GAAACGTGGCTGGCCTGGTTCACCACGCGGGAAACGGTCTGATAAGAGACACCGGC
ATACTCTGCGACATCGTATAACGTTACTGGTTTCACATTCACCACCCTGAATTGACTC
TCTTCCGGGCGCTATCATGCCATACCGCGAAAGGTTTTGCACCATTCGATGGTGTCG
GAATTTCGGGCAGCGTTGGGTCCTGGCCACGGGTGCGCATGATCTAGAGCTGCCTCG
CGCGTTTCGGTGATGACGGTGAAAACCTCTGACACATGCAGCTCCCGGAGACGGTC
ACAGCTTGTCTGTAAGCGGATGCCGGGAGCAGACAAGCCCGTCAGGGCGCGTCAGC
GGGTGTTGGCGGGTGTCGGGGCGCAGCCATGACCCAGTCACGTAGCGATAGCGGAG
TGTATACTGGCTTAACTATGCGGCATCAGAGCAGATTGTACTGAGAGTGCACCATAT
GCGGTGTGAAATACCGCACAGATGCGTAAGGAGAAAATACCGCATCAGGCGCTCTT
CCGCTTCCTCGCTCACTGACTCGCTGCGCTCGGTCGTTCGGCTGCGGCGAGCGGTAT
CAGCTCACTCAAAGGCGGTAATACGGTTATCCACAGAATCAGGGGATAACGCAGGA
AAGAACATGTGAGCAAAAGGCCAGCAAAAGGCCAGGAACCGTAAAAAGGCCGCGT
TGCTGGCGTTTTTCCATAGGCTCCGCCCCCCTGACGAGCATCACAAAAATCGACGCT
CAAGTCAGAGGTGGCGAAACCCGACAGGACTATAAAGATACCAGGCGTTTCCCCCT
GGAAGCTCCCTCGTGCGCTCTCCTGTTCCGACCCTGCCGCTTACCGGATACCTGTCC
GCCTTTCTCCCTTCGGGAAGCGTGGCGCTTTCTCATAGCTCACGCTGTAGGTATCTCA
GTTCGGTGTAGGTCGTTCGCTCCAAGCTGGGCTGTGTGCACGAACCCCCCGTTCAGC
CCGACCGCTGCGCCTTATCCGGTAACTATCGTCTTGAGTCCAACCCGGTAAGACACG
ACTTATCGCCACTGGCAGCAGCCACTGGTAACAGGATTAGCAGAGCGAGGTATGTA
GGCGGTGCTACAGAGTTCTTGAAGTGGTGGCCTAACTACGGCTACACTAGAAGGAC
AGTATTTGGTATCTGCGCTCTGCTGAAGCCAGTTACCTTCGGAAAAAGAGTTGGTAG
CTCTTGATCCGGCAAACAAACCACCGCTGGTAGCGGTGGTTTTTTTGTTTGCAAGCA
GCAGATTACGCGCAGAAAAAAAGGATCTCAAGAAGATCCTTTGATCTTTTCTACGGG
GTCTGACGCTCAGTGGAACGAAAACTCACG
>GFP-H1.8
(SEQ ID NO: 14)
MAPKKAVAAPEGGNKENAAVKGSSKVKVKRKSIKLVKTQSHPPTLSMVVEVLKKNTE
RKGTSVQAIRTRILSAHPTVDPLRLKFLLRTALNKGLEKGILIRPLNSSATGATGRFKLAK
PVKTTKAGKENVASENVDPNAEQETQKKAPKKEKKAKTEKEPKGEKTKAVAKKAKED
SDEKPKVAKSKKDKEAKEVDKANKEAKEVDKANKEAKEVDKAPAKKPKAKTEAAKA
EGGGKAKKEPPKAKAKDVKAQKDSTDEGAPVKAGKKGKKVTNGGGGSGGGGSMSKG
EELFTGVVPILVELDGDVNGHKFSVRGEGEGDATNGKLTLKFICTTGKLPVPWPTLVTTL
TYGVQCFSRYPDHMKRHDFFKSAMPEGYVQERTISFKDDGTYKTRAEVKFEGDTLVNR
IELKGIDFKEDGNILGHKLEYNFNSHNVYITADKQKNGIKANFKIRHNVEDGSVQLADH
YQQNTPIGDGPVLLPDNHYLSTQSVLSKDPNEKRDHMVLLEFVTAAGITHGMDELYK

All histones are purified with the method described previously (Zierhut et al., 2014). Bacterial expressed X. laevis H2A, H2B, H3.2, and H4 are purified from inclusion bodies. His-tagged histones (H2A, H3.2, and H4) or untagged H2B expressed in bacteria are resolubilized from the inclusion bodies by incubation with the 6 M guanidine HCl. For His-tagged histones, the solubilized His-tagged histones are purified using Ni-NTA beads (Qiagen). For untagged H2B, resolubilized histones are purified using the MonoS column (Cytiva) under denaturing conditions before H2A-H2B dimer formation. To reconstitute H3-H4 tetramer and H2A-H2B dimer, the denatured histones are mixed at an equal molar ratio and dialyzed to refold histones by removing the guanidine. His-tags are removed by TEV protease treatment, and H3-H4 tetramer and H2A-H2B dimer are isolated through HiLoad 16/600 Superdex 75 column (Cytiva). The fractions containing H3-H4 tetramer and H2A-H2B dimer are concentrated using Amicon 10K, flash frozen, and stored at −80° C. Listed below are the amino acid sequences of purified recombinant X. laevis H2A, H2B, H3.2, and H4.

>X. laevis H2A
(SEQ ID NO: 15)
SGRGKQGGKTRAKAKTRSSRAGLQFPVGRVHRLLRKGNYAERVGAGAPV
YLAAVLEYLTAEILELAGNAARDNKKTRIIPRHLQLAVRNDEELNKLLG
RVTIAQGGVLPNIQSVLLPKKTESSKSAKSK
>X. laevis H2B
(SEQ ID NO: 16)
MPEPAKSAPAPKKGSKKAVTKTQKKDGKKRRKTRKESYAIYVYKVLKQV
HPDTGISSKAMSIMNSFVNDVFERIAGEASRLAHYNKRSTITSREIQTA
VRLLLPGELAKHAVSEGTKAVTKYTSAK
>X. laevis H3.2
(SEQ ID NO: 17)
ARTKQTARKSTGGKAPRKQLATKAARKSAPATGGVKKPHRYRPGTVALR
EIRRYQKSTELLIRKLPFQRLVREIAQDFKTDLRFQSSAVMALQEASEA
YLVALFEDTNLCAIHAKRVTIMPKDIQLARRIRGERA
>X. laevis H4
(SEQ ID NO: 18)
SGRGKGGKGLGKGGAKRHRKVLRDNIQGITKPAIRRLARRGGVKRISGL
IYEETRGVLKVFLENVIRDAVTYTEHAKRKTVTAMDVVYALKRQGRTLY
GFGG

Nucleosome Isolation from Xenopus Egg Extracts Chromosomes

Nucleosomes are isolated from Xenopus egg extracts chromosomes with the previously described method (Arimura et al., 2021). The cytostatic factor (CSF) metaphase-arrested Xenopus laevis egg extracts are prepared with the method described previously (Murray, 1991). To prevent spontaneous cycling of egg extracts, 0.1 mg/ml cycloheximide is added to the CSF extract. H1.8-GFP are added to the CSF extract at a final concentration of 650 nM, which is an equal to the concentration with endogenous H1.8 (Wühr et al., 2014). For interphase chromosome preparation, Xenopus laevis sperm nuclei (final concentration 2000/μl) are added to 6 ml of CSF extracts, which are incubated for 90 min at 20° C. after adding 0.3 mM CaCl₂), which releases CSF extracts into interphase. To monitor spindle assembly, Alexa594-labeled-bovine brain tubulin (final concentration 19 nM) is added to the extract during the incubation. For the metaphase sperm chromosome preparation, cyclin B 490 (final concentration 24 μg/ml) and 3 ml fresh CSF extract is added to 6 ml of the extract containing interphase sperm nuclei prepared with the method described above. The extracts are incubated for 60 min at 20° C., during which each tube is gently mixed every 10 min. The Animal husbandry and protocol approved by Rockefeller University's Institutional Animal Care and Use Committee are followed. To crosslink the Xenopus egg extracts chromosomes, nine times the volume of ice-cold buffer XL (80 mM PIPES-KOH [pH 6.8]. 15 mM NaCl, 60 mM KCl, 30% glycerol, 1 mM EGTA, 1 mM MgCl₂. 10 mM β-glycerophosphate, 10 mM sodium butyrate, 2.67% formaldehyde) is added to the interphase or metaphase extract with chromosomes, which is further incubated for 60 min on ice. These fixed chromosomes are collected using centrifuge at 3,300 (2,647 rcf) rpm at 4° C. for 40 min using JS 5.3 rotor in Avanti J-26S (Beckman Coulter). Pellets containing fixed chromosomes are resuspended with 10 ml of buffer SC (80 mM HEPES-KOH [pH 7.4]. 15 mM NaCl. 60 mM KCl. 1.17 M sucrose, 50 mM glycine, 0.15 mM spermidine, 0.5 mM spermine, 1.25× complete EDTA-free Protease Inhibitor Cocktail (Roche), 10 mM β-glycerophosphate. 10 mM sodium butyrate, 1 mM EGTA, 1 mM MgCl₂) layered on 3 ml of fresh buffer SC in 14 ml centrifuge tubes (Falcon, #352059) and spun at 3,300 (2,647 rcf) rpm at 4° C. for 40 min using JS 5.3 rotor in Avanti J-26S (Beckman Coulter). The chromosomes are collected from the bottom of the centrifuge tube and resuspended with buffer SC. Chromosomes are pelleted by centrifugation at 5.492 rpm (2,500 rcf) using SX241.5 rotor in Allegron X-30R (Beckman Coulter). The chromosome pellets are resuspended with 200 μL of buffer SC. To digest chromatin, 0.1 U/μL of MNase (Worthington Biochemical Corporation) and CaCl₂) are added to 7.4 mM, and the mixture is incubated at 4° C. for 6 h. MNase reaction is stopped by adding 900 μL MNase stop buffer (15 mM HEPES-KOH [pH 7.4], 150 mM KCl, 5 mM EGTA, 10 mM β-glycerophosphate. 10 mM sodium butyrate, 5 mM DTT). The soluble fractions released by MNase were isolated by taking supernatants after centrifugation at 13,894 rpm (16,000 rcf) at 4° C. for 30 min using SX241.5 rotor in Allegron X-30R (Beckman Coulter). The supernatants are collected and layered onto the 10-22% linear sucrose gradient solution with buffer SG (15 mM HEPES-KOH [pH 7.4], 50 mM KCl, 10-22% sucrose, 10 μg/ml leupeptin, 10 μg/ml pepstatin, 10 μg/ml chymostatin, 10 mM sodium butyrate, 10 mM β-glycerophosphate. 1 mM EGTA) and spun at 32,000 rpm (max 124.436 rcf) and 4° C. for 13 h using SW55Ti rotor in Optima L80 (Beckman Coulter). The samples are fractionated from the top of the sucrose gradient. The concentration of the H1.8 in each fraction is determined by western blot. 20 μL each of sucrose gradient fraction is incubated at 95° C. under the existence of 1% Sodium dodecyl sulfate (SDS) applied for SDS-PAGE with 4-20% gradient SDS-PAGE gel (Bio-rad). The proteins are transferred to the nitrocellulose membrane (Cytiva) from the SDS-PAGE gel using TE42 Tank Blotting Units (Hoefer) at 15 V. 4° C. for 4 h. As primally antibodies, 1 μg/ml of mouse monoclonal Anti-GFP Antibody sc-9996 (Santa cruz) and as secondary antibodies, IR Dye 800CW goat anti-mouse IgG (Li-Cor 926-32210; 1:15,000) are used. The images are taken with Odyssey Infrared Imaging System (Li-Cor).

### Recombinant Nucleosome Reconstitution

For mono-nucleosomes, the 193 bp 601 DNA fragment is amplified by PCR reaction (Arimura et al., 2012; Lowary and Widom, 1998). For the biotinylated-poly-nucleosomes, pAS696 containing the 19-mer of the 200 bp 601 nucleosome positioning sequence is digested using HaeII, DraI. EcoRI, and Xbal. Both ends of the 19-mer of the 200 bp 601 DNA are labeled with biotin by Klenow fragment (NEB) with biotin-14-dATP (Guse et al., 2012). The nucleosomes are assembled with the salt dialysis method (Guse et al., 2012). Purified DNAs are mixed with H3-H4 and H2A-H2B, transferred into a dialysis cassette, and placed into a high salt buffer (10 mM Tris-HCl [pH 7.5], 1 mM EDTA, 2 M NaCl, 5 mM β-mercaptocthanol, and 0.01% Triton X-100). Using a peristaltic pump, the high salt buffer is exchanged with low salt buffer (10 mM Tris-HCl [pH 7.5], 1 mM EDTA. 50 mM NaCl, 5 mM β-mercaptoethanol, 0.01% Triton X-100) at roughly 2 ml/min for overnight at 4° C. In preparation for cryo-EM image collection, the dialysis cassette containing the sample is then placed in a buffer containing 10 mM HEPES-HCl (pH 7.4) and 30 mM KCl, and dialyzed for 48 h at 4° C. Listed below are the DNA sequences of 193 bp 601 DNA, PCR primers and 19-mer 200 bp 601 DNA.

>193 bp 601 DNA
(SEQ ID NO: 19)
ATCGGACCCTATCGCGAGCCAGGCCTGAGAATCCGGTGCCGAGGCCGCT
CAATTGGTCGTAGACAGCTCTAGCACCGCTTAAACGCACGTACGCGCTG
TCCCCCGCGTTTTAACCGCCAAGGGGATTACTCCCTAGTCTCCAGGCAC
GTGTCAGATATATACATCCAGGCCTTGTGTCGCGAAATTCATAGAT
>PCR primer (top) for 193 bp 601 DNA
(SEQ ID NO: 20)
ATCGGACCCTATCGCGAGCCAGGCCTGAGAATCCGGT
>PCR primer (bottom) for 193 bp 601 DNA
(SEQ ID NO: 21)
ATCTATGAATTTCGCGACACAAGGCCTGGATGTATATATCTGACAC

### In Vitro Reconstitution of H1.8-GFP Bound Nucleosome

Reconstituted nucleosome with 193 bp 601 DNA is dialyzed against buffer XL. Dialyzed nucleosome is mixed with H1.8-GFP at a 2:3 molar ratio under the existence of 0.001% poly-a-glutamic acid and incubated for 30 min at 37° C. The H1.8-GFP bound nucleosome is crosslinked with the addition of a 0.5-time volume of buffer XL containing 3% formaldehyde and with 60 min incubation on ice. The crosslink reaction is quenched with buffer Q (30 mM HEPES-KOH (pH 7.4). 150 mM KCl, 1 mM EGTA, 10 ng/μL leupeptin. 10 ng/μL pepstatin. 10 ng/μL chymostatin, 10 mM Sodium Butyrate, 10 mM b-glycerophosphate, 400 mM glycine, 1 mM MgCl₂, 5 mM DTT). The quenched sample is layered onto the 10-25% linear sucrose gradient solution with buffer SG and spun at 32000 rpm (max 124.436 rcf) and 4° C. for 13 h using SW55Ti rotor in Optima L80 (Beckman Coulter). The centrifuged samples are fractionated from the top of the sucrose gradient. The concentration of H1.8-GFP bound nucleosome in each fraction is calculated based on the 260 nm light absorbance detected by Nanodrop 2000 (Thermo Scientific).

### Preparation of Biotin-Polynucleosome Bound Nano-Magnetic Beads

60 fmol of Absolute Mag streptavidin nano-magnetic beads (CD bioparticles: WHM-X047, 50 nM size) are mixed with 100 μL of EM buffer A (10 mM HEPES-KOH (pH 7.4), 30 mM KCl, 1 mM EGTA. 0.3 ng/μL leupeptin, 0.3 ng/μL pepstatin, 0.3 ng/μL chymostatin. 1 mM Sodium Butyrate, 1 mM beta-glycerophosphate, 1 mM MgCl₂, 2% trehalose, 0.2% 1,6-hexanediol). The beads are collected with incubation on two pieces of 40×20 mm N52 neodymium disc magnets (DIYMAG: D40×20-2P-NEW) at 4° C. for 30 min and resuspend with 120 μL EM buffer A. 60 μL of 34 nM nucleosome arrays formed on the biotinylated 19-mer 200 bp 601 DNA are mixed with beads and rotated at 20° C. for 2 hours. To remove unbound nucleosomes, the biotin-poly-nucleosome-bound nano-magnetic beads are collected after 40 min of incubation on the N52 neodymium disc magnets and resuspended with 300 μL EM buffer containing 10 μM biotin. 100 μL of the biotin-poly-nucleosome bound nano-magnetic beads solution are incubated on the N52 neodymium disc magnets 30 minutes and resuspended with 20 μL EM buffer A (theoretical beads concentration: 1 nM). 3 μL of 10 nM biotin-poly-nucleosome bound nano-magnetic beads solution is added onto a glow discharged Quantifoil Gold R 1.2/1.3 300 mesh grid (Quantifoil). The samples are vitrified under 100% humidity, 20 seconds incubation, and 5 seconds blotting time using the Vitrobot Mark IV (FEI).

### Preparation of Spacer Protein Conjugated Nano-Magnetic Beads

25 fmol of Absolute Mag streptavidin nano-magnetic beads (CD bioparticles: WHM-X047) are transferred to 0.5 ml of protein LoBind tube (Eppendorf) and spun at 12,032 rpm (12,000 rcf) at 4° C. for 10 min using SX241.5 rotor in Allegron X-30R (Beckman Coulter). The beads accumulated at the bottom of the tube are resuspended with 150 μL of EM buffer B (10 mM HEPES-KOH (pH 7.4). 30 mM KCl, 1 mM EGTA, 10 ng/μL leupeptin. 10 ng/μL pepstatin, 10 ng/u L chymostatin, 1 mM Sodium Butyrate, and 1 mM beta-glycerophosphate) and 200 pmol of biotin-3HB-SPYcatcher003 (for 3HB-30 nm-SAH nano-magnetic beads and 3HB-60 nm-SAH nano-magnetic beads preparation) or biotin-30 nm-SAH-SPYcatcher003 (for 30 nm-SAH×2 nano-magnetic beads preparation) are added. After 12 hours of incubation at 4° C., 5 μM biotin is added to fill the free biotin binding pockets on the streptavidin. To wash the beads, the beads are collected with centrifugation and resuspended with 200 μL of EM buffer B. This washing step is repeated once again, and the beads are resuspended with 180 μL of EM buffer B. 200 pmol of mono-SPYtag-avidin tetramer is added to the beads and incubated at 4° C. for 12 hours. To wash the beads, the beads are collected with centrifugation and resuspended with 200 μL of EM buffer B. This washing step is repeated once again, and the beads are resuspended with 150 μL of EM buffer B. 800 pmol of biotin-30 nm-SAH-SPYcatcher003 (for 3HB-30 nm-SAH nano-magnetic beads and 30 nm-SAHx2 nano-magnetic beads preparation) or biotin-60 nm-SAH-SPYcatcher003 (for 3HB-60 nm-SAH nano-magnetic beads preparation) is added to the beads and incubated at 4° C. for 12 hours. To wash the beads, the beads are collected with centrifugation and resuspended with 200 μL of EM buffer B. This washing step is repeated once again, and the beads are resuspended with 25 μL beads storage buffer (8 mM Na₂HPO₄, 2 mM KH₂PO₄. 137 mM NaCl, 0.05% NaN₃. 0.01% Tween 20, and 1 mg/ml BSA [pH 7.4]) and store at 4° C. for several weeks.

### Addition of Tandem GFP Nanobodies to Spacer Protein Conjugated Nano-Magnetic Beads

10 fmol of spacer protein conjugated nano-magnetic beads are mixed with 80 pmol of His₆-SPYtag-tandem GFP nanobodies in 200 μL of PBS containing about 15-30% glycerol and incubated at 4° C. for 12 hours. To wash the beads, the beads are collected with centrifugation and resuspended with 200 μL of PBS containing about 15-30% glycerol. This washing step is repeated once again, and the beads are resuspended with 100 μL beads PBS containing 15˜30% glycerol and stored at 4° C. for several days (theoretical beads concentration: 100 pM).

### MagIC-Cryo-EM of In Vitro Reconstituted H1.8-GFP Bound Nucleosome Using Nano-Magnetic Beads

4 fmol of tandem GFP nanobodies conjugated 3HB-30 nm-SAH or 30 nm-SAH×2 nano-magnetic beads are mixed with 10 pmol (2.8 μg) of in vitro reconstituted H1.8-GFP bound nucleosome in 100 μL of PBS containing about 15-30% glycerol and incubated at 4° C. for 12 hours. To wash the beads, the beads are collected with centrifugation at 13,894 rpm (16,000 rcf) at 4° C. for 20 min using SX241.5 rotor in Allegron X-30R (Beckman Coulter) and resuspended with 200 μL of PBS containing about 15-30% glycerol. This washing step is repeated once again, and the beads are resuspended with 100 μL of EM buffer C (10 mM HEPES-KOH (pH 7.4), 30 mM KCl. 1 mM EGTA, 10 ng/μL leupeptin, 10 ng/μL pepstatin, 10 ng/μL chymostatin, 1 mM sodium butyrate, 1 mM beta-glycerophosphate, 1.2% trehalose, and 0.12% 1,6-hexanediol). This washing step is repeated once again, and the beads arc resuspended with about 100-200 μL of EM buffer C (theoretical beads concentration: 20˜40 PM).

MagIC-Cryo-EM with a Minimum Amount of In Vitro Reconstituted H1.8-GFP Bound Nucleosome

0.5 fmol of tandem GFP nanobodies conjugated 3HB-60 nm-SAH nano-magnetic beads mixed with 0.1, 0.5, and 1 pmol (28, 140, 280 ng) of H1.8-GFP bound nucleosome in 100 μL PBS (1, 5, and 10 nM) containing 5% glycerol and 0.01% tween-20 and incubated on nutating mixer 4° C. for 4 hours. To wash the beads, the beads are collected with centrifugation at 13,894 rpm (16,000 rcf) at 4° C. for 20 min using SX241.5 rotor in Allegron X-30R (Beckman Coulter) and resuspended with 200 μL of PBS containing 15% glycerol and 0.01% tween-20. This washing step is repeated once again, and the beads are resuspended with 100 μL of EM buffer C. This washing step is repeated once again, and the beads are resuspended with 15 μL of EM buffer C (theoretical beads concentration: 33 PM).

MagIC-Cryo-EM of H1.8-GFP Bound Nucleosome Isolated from Xenopus Egg Extract Using Nano-Magnetic Beads

To remove the proteins that nonspecifically bind to spacer module proteins from chromosomal nucleosome fractions, chromosomal nucleosome fractions isolated from the metaphase chromosome are washed by decoy magnetic beads that have biotin-3HB-SPYcatcher003 and biotin-30 nm-SAH-SPYcatcher003 on Dynabeads M-280 Streptavidin (Thermo Fisher), which is 2.8 μm beads. To prepare decoy magnetic beads, 100 μL of 10 mg/ml Dynabeads M-280 Streptavidin (Thermo Fisher) are mixed with 250 pmol of Biotin-3HB-SPYcatcher003 in 200 μL of PBS containing 15% glycerol and are rotated at 4° C. for 4 hours. The rotated Dynabeads are collected on the magnetic rack MCP-S (Dynal) and washed twice with PBS containing 15% glycerol and 0.01% tween-20. The washed beads are mixed with 250 pmol of mono-SPYtag-avidin tetramer in 250 μL of PBS containing 15% glycerol and are rotated at 4° C. for 4 hours. The rotated Dynabeads are collected on the magnetic rack MCP-S (Dynal) and washed twice with PBS containing 15% glycerol and 0.01% tween-20. The washed beads are mixed with 1000 pmol of biotin-30 nm-SAH-SPYcatcher003 in 250 μL of PBS containing 15% glycerol and are rotated at 4° C. for 4 hours. The rotated Dynabeads are collected on the magnetic rack MCP-S (Dynal) and washed twice with PBS containing 15% glycerol and 0.01% tween-20. The washed beads are mixed with 1000 pmol of biotin-30 nm-SAH-SPYcatcher003 in 250 μL of PBS containing 15% glycerol and 0.01% tween-20. The washed decoy magnetic beads are stored in 100 μL of PBS containing 15% glycerol and 0.01% tween-20 at 4° C. for several weeks. 10 μL of decoy magnetic beads (theoretically contains 10 mg/ml Dynabeads M-280) are mixed with the nucleosome fraction isolated from metaphase chromosomes that contains 0.4 pmol H1.8-GFP in 250 μL PBS containing 5% glycerol and 0.01% tween-20 and rotated at 4° C. for 4 hours. The decoy magnetic beads are removed with centrifugation at 13.894 rpm (16.000 rcf) at 4° C. for 10 min using SX241.5 rotor in Allegron X-30R (Beckman Coulter). The supernatant is mixed with 0.5 fmol of tandem GFP nanobodies conjugated 3HB-30 nm-SAH nano-magnetic beads and incubated on nutating mixer 4° C. for 4 hours. To wash the beads, the beads are collected with centrifugation at 13,894 rpm (16.000 rcf) at 4° C. for 20 min using SX241.5 rotor in Allegron X-30R (Beckman Coulter) and resuspended with 200 μL of PBS containing 15% glycerol and 0.01% tween-20. This washing step is repeated once again, and the beads are resuspended with 100 μL of EM buffer C. This washing step is repeated once again, and the beads are resuspended with 20 μL of EM buffer C (theoretical beads concentration: 25 PM).

### Magnetic Concentration of Nano-Magnetic Beads on Cryo-EM Grid

A glow discharged Quantifoil gold R1.2/1.3 grid (Quantifoil) that has monolayer graphene (Han et al., 2020) is anchored with a pair of sharp tweezers that is attached to the Vitrobot Mark IV (FEI). 4 μL of about 10-1000 pM nucleosome bound nano-magnetic beads solution is applied onto the grid at outside the Vitrobot. The grid is incubated on the 40×20 mm N52 neodymium disc magnets for 1 min in a high humidity chamber. Then, the tweezers anchoring the grid are attached to the Vitrobot Mark IV (FEI), and the grid is vitrified under 100% humidity and 2 sec blotting time at room temperature.

### Cryo-EM Data Collection and Image Processing

Grids are imaged on a Talos Arctica (FEI) installed with a K2 Camera (GATAN) and a field emission gun operating at 200 kV or Titan Krios (FEI) installed with a K2 Camera (GATAN) and a 300 kV field emission gun. Movie frames are motion-corrected and dose-weighted patch motion correction in CryoSPARC v3.3 with output Fourier cropping factor 1/2. Particles arc picked by Topaz v0.2.3 with around 2000 manually picked nucleosome-like particles as training models (Bepler et al., 2019). Picked particles are extracted using CryoSPARC v3.3 (extraction box size=around 400 Å) (Punjani et al., 2017). Extracted particles are applied for 2D classification with 100 classes using CryoSPARC v3.3. Using 2D classification results, particles are split into the nucleosome-like groups and the non-nucleosome-like groups. Four 3D initial models are generated for both groups with ab initio reconstruction of CryoSPARC v3.3 (Class similarity=0). One nucleosome-like model is selected and used as a given model of 3D classification with all four of the “decoy” classes generated from the non-nucleosome-like group. After the first round of 3D classification, the particles assigned to the “decoy” classes are removed, and the remaining particles are applied for the second round of 3D classification in the same setting as the first round. These steps are repeated until about 90-5% of particles are classified as a nucleosome-like class. To isolate the nucleosome class that has visible H1.8 density, four to six 3D references are generated with ab initio reconstruction of CryoSPARC v3.3 using purified nucleosome-like particles (Class similarity=0.9). Refined particles are further purified with the heterogeneous refinement using an H1.8-visible class and an H1.8-invisible class as decoys. The classes with reasonable extra density are selected and refined with homogeneous refinement. The final resolution is determined with the gold stand FSC threshold (FSC=0.143).

### Example 2: Trial MagIC-Cryo-EM of Biotin-Poly-Nucleosome

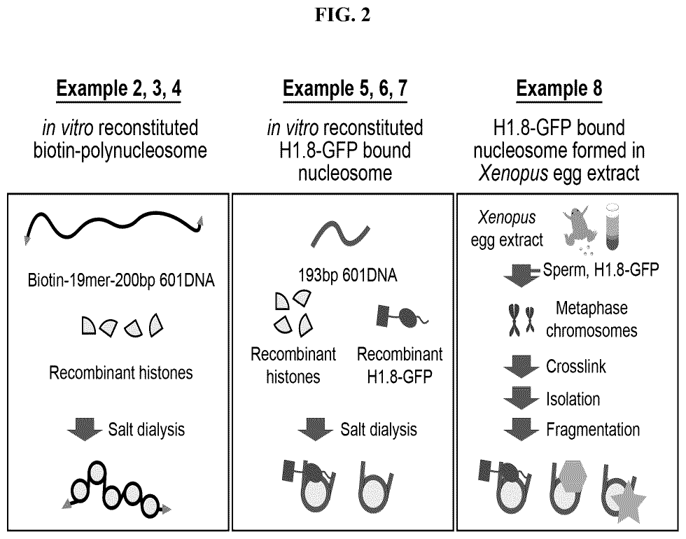

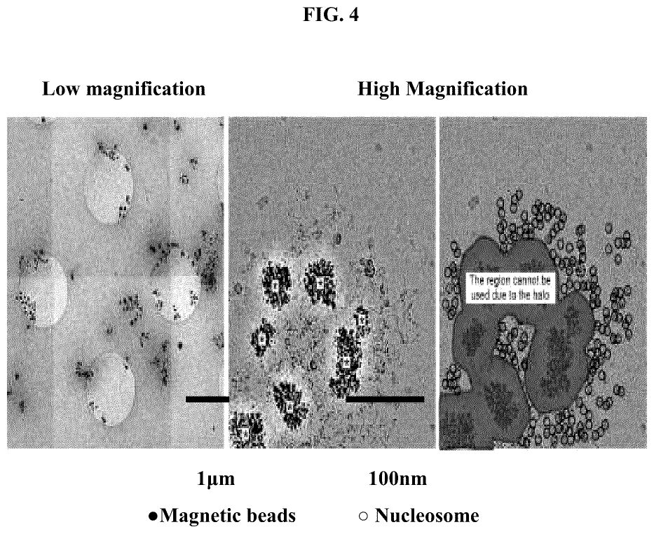

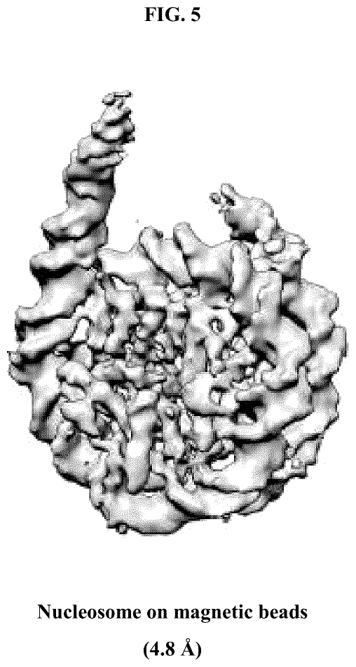

Although nano-magnetic beads had been used for taking snapshot cryo-EM micrograph of virus pseudo particles (Bonnafous et al., 2010), it has never been used for single particle analysis to reconstitute a 3D structure of protein complexes. To test if nano-magnetic beads can be used for cryo-EM single particle analysis, the nano-magnetic beads capturing biotin-poly-nucleosome are prepared. The biotin-poly-nucleosomes are reconstituted in vitro using biotinylated-19 mer 601 DNA and recombinant histone complexes (FIG. 2 left). The reconstituted biotin-poly-nucleosomes are captured by Absolute Mag streptavidin magnetic beads (FIG. 3 left). On the low magnification (x2600) cryo-EM micrograph, magnetic beads are clearly visible as black dots, while nucleosomes captured by the beads are not visible at this magnification (FIG. 4 left). In the conventional cryo-EM data collection, low magnification micrographs are taken, and the locations that will be used for high magnification data collection are picked on the low magnification micrographs. The visibility of the magnetic beads at low magnification provides a significant benefit in finding the target particles before taking the high magnification micrograph, which allows users to take more usable images in the limited cryo-EM machine time. In the high magnification (x28000) micrograph, the magnetic beads are seen as strong blob-shaped signals, and many nucleosomes are seen around the beads (FIG. 4 middle). However. 20-30 nm white halo-shaped noise appears around each bead. Due to the noise, only the nucleosomes more than 30 nm away from beads are used for the structural analysis (FIG. 4 right). Using the nucleosome particles outside the halo-shaped noise, the 3D structure of the nucleosomes is reconstituted at 4.8 Å resolution (FIG. 5). This suggests that the nano-magnetic beads can be used for cryo-EM single particle analysis.

In this example, many of the nucleosomes are identified outside the halo-shaped noise because the 19mer-poly-nucleosome arrays extend outside of the noise. For other common non-polymer target particles, such as isolated proteins, however, this noise will prevent high-resolution cryo-EM single particle analyses because the substantial fraction of target particles will be partially or completely covered with the halo-shaped noise. This issue is solved by using spacer protein, which will be described in Examples 5 and 6.

### Example 3: Concentrating Magnetic Beads on Cryo-EM Grid by a Magnetic Force

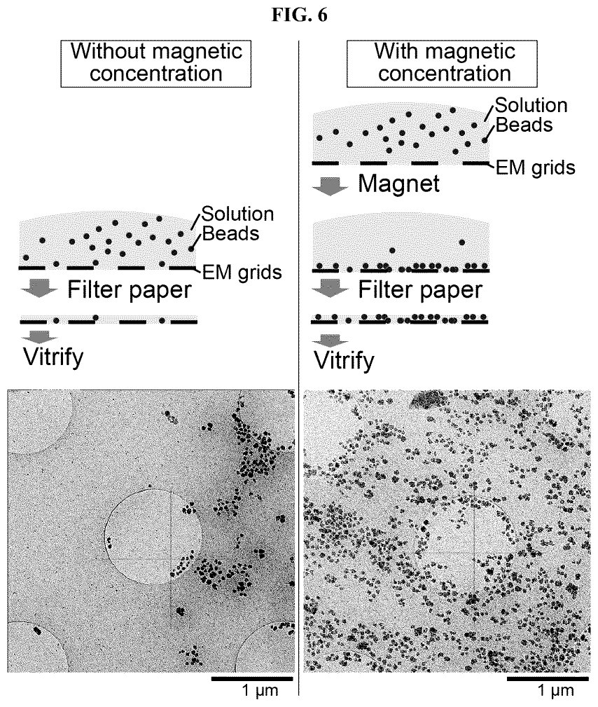

In the conventional cryo-EM grid preparations, 2-4 μL of aqueous samples are loaded onto a grid, and then most of the liquid that contains target particles is removed by a filter paper to form thin ice on the grid. Since the target particles are dispersed in the liquid, this procedure loses most of the target particles in the liquid (FIG. 6 left). To prevent sample loss during this process, the magnetic beads are concentrated on cryo-EM grids by incubating the grid for 1 min using a magnet placed in a humidity chamber (FIG. 6 right). Compared to the cryo-EM micrograph frozen without magnetic concentration (FIG. 6 left), the micrograph frozen after the magnetic concentration contains more particles on the grid (FIG. 6 right). This suggests that magnetic beads are efficiently concentrated on cryo-EM grids with the magnetic concentration.

### Example 4: Using Monolayer Graphene Grid for Magnetic Concentration

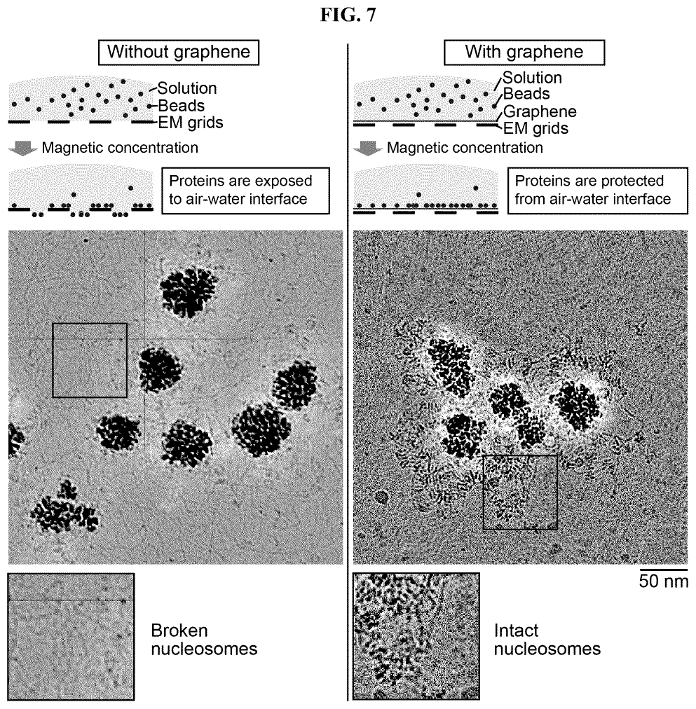

Although the magnetic concentration is helpful in preventing protein loss during the freezing step, pulling beads with a magnetic may expose proteins to the air-water interface, which is known to denature proteins during freezing (D'Imprima et al., 2019). Indeed, the nucleosomes on the magnetic beads are significantly damaged after the magnetic concentration on the commercially available quantifoil holy carbon grid R1.2/1.3 (FIG. 7 left). To solve this issue, monolayer graphene was attached to the surface of the grid with previously reported method (Han et al., 2020). With a monolayer graphene-coated grid, nucleosomes on the magnetic beads are intact after the magnetic concentration (FIG. 7 right), as the monolayer graphene is expected to cover the air-water interface and prevent the proteins from being exposed to the air.

### Example 5: MagIC-Cryo-EM of In Vitro Reconstituted H1.8-GFP Bound Nucleosome Using 3HB-30 nm-SAH Nano Magnetic Beads

The cryo-EM of the nano-magnetic beads capturing biotin-poly-nucleosomes (Example 2) suggest that nano-magnetic beads generate 20-30 nm halo-shaped noise. This noise will be a problem for non-polymer target particles captured but kept within the noise region of the beads. To overcome this issue, spacer modules are attached to the Absolute Mag streptavidin magnetic beads (FIG. 3: 3HB-30 nm-SAH GFP nanobody beads). In this example, two types of spacer modules are tandemly attached to beads. The first spacer module attached to Absolute Mag streptavidin magnetic beads is the biotin-3HB-SPYcatcher003, which contains a computationally designed 11 Å long monomeric triple helical bundle protein (Huang et al., 2014). Then, a mono-SPYtag-avidin tetramer that has a single SPYtag003 on each streptavidin tetramer is attached to the top of the biotin-3HB-SPYcatcher003 using the covalent interaction between SPYcatcher003 and SPYtag003 (Keeble et al., 2019). Since a single mono-SPYtag-avidin tetramer binds four biotin molecules, protein binding capability on each bead increases four-fold. The second spacer protein is biotin-30 nm-SAH-SPYcatcher003, which contains a 30 nm single alpha-helix obtained from Trichomonas Vaginalis (Sivaramakrishnan and Spudich, 2011). To the second spacer module biotin-30 nm-SAH-SPYcatcher003, a target capturing module, His₆-SPYtag-tandem GFP nanobody, which captures GFP, is attached.

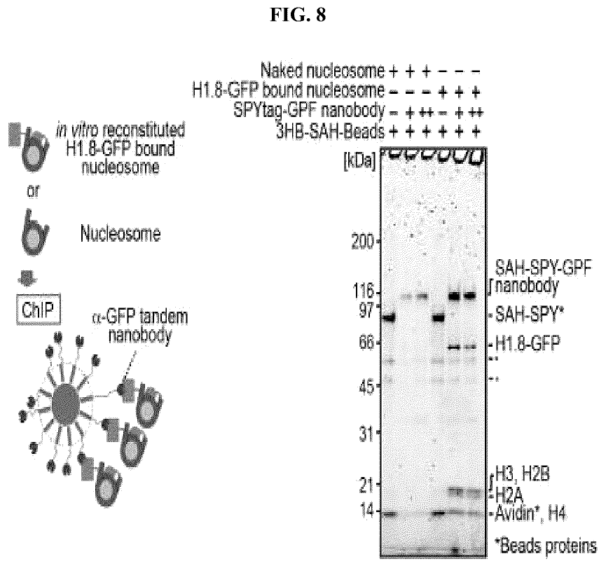

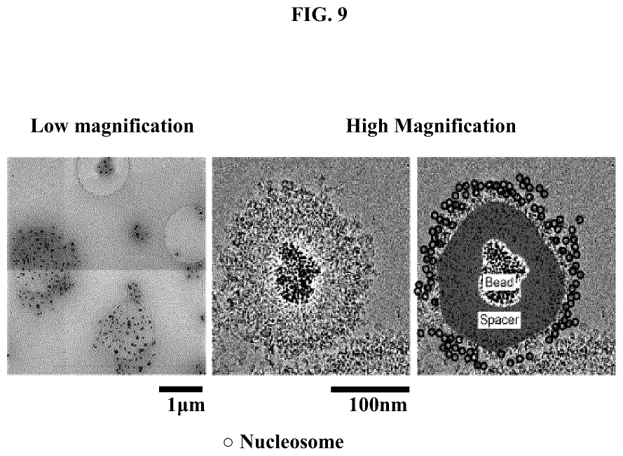

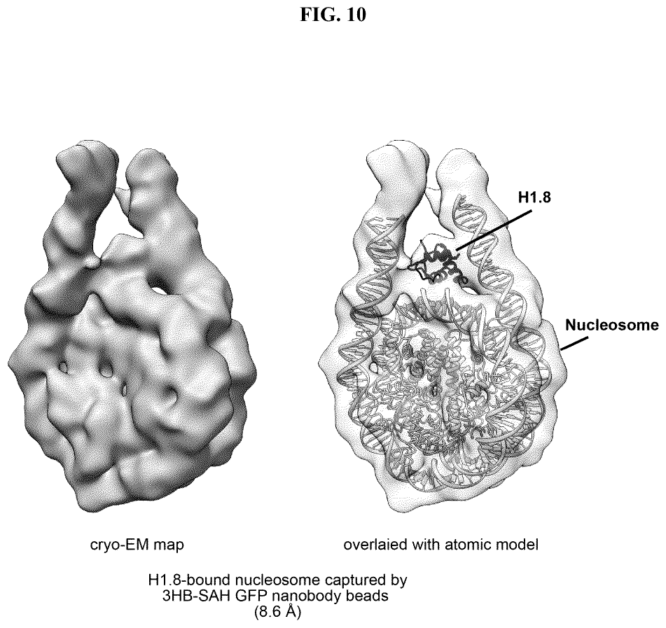

To test these beads with the spacer modules and the target capturing module, nucleosomes and H1.8-GFP bound nucleosomes are reconstituted in vitro and mixed with the 3HB-30 nm-SAH beads with or without His₆-SPYtag-tandem GFP nanobody (FIG. 8 left). The nano-magnetic beads are collected and washed with centrifugation and analyzed with SDS-PAGE. The H1.8-GFP is specifically isolated with nano-magnetic beads only when the His₆-SPYtag-tandem GFP nanobody existed, suggesting that the level of non-specific binding is low (FIG. 8 right). Furthermore, histone proteins (H3, H2A. H2B) are copurificd with H1.8-GFP, suggesting that the beads can isolate GFP-contained complexes. 4 μL of 40 pM 3HB-30 nm-SAH GFP nanobody beads that capture a maximum of 112 ng of H1.8-GFP bound nucleosomes are loaded onto a cryo-EM grid coated with a monolayer graphene, concentrated by a magnetic force, and plunge frozen. On a low magnification (x2.600) cryo-EM micrograph, circular rings are seen around each magnetic bead, suggesting that spacer proteins successfully provide spaces around the beads, and each bead is well separated (FIG. 9 left). On high magnification (x28,000) cryo-EM micrograph, a 40-50 nm circular layer around each bead covers the halo-shaped noise (FIG. 9 middle and right). The nucleosome-like particles outside the spacer region are picked using machine learning-based picking software Topaz (Bepler et al., 2019) and used for 3D structure reconstruction. The 3D structure reconstituted from 1,131 micrographs has an extra EM density around the nucleosome dyad (FIG. 10 arrow), demonstrating that structural analysis of the low concentration protein complex is possible with the MagIC-cryo-EM method.

### Example 6: MagIC-Cryo-EM of In Vitro Reconstituted H1.8-GFP Bound Nucleosome Using 30 nm-SAHx2 Nano Magnetic Beads

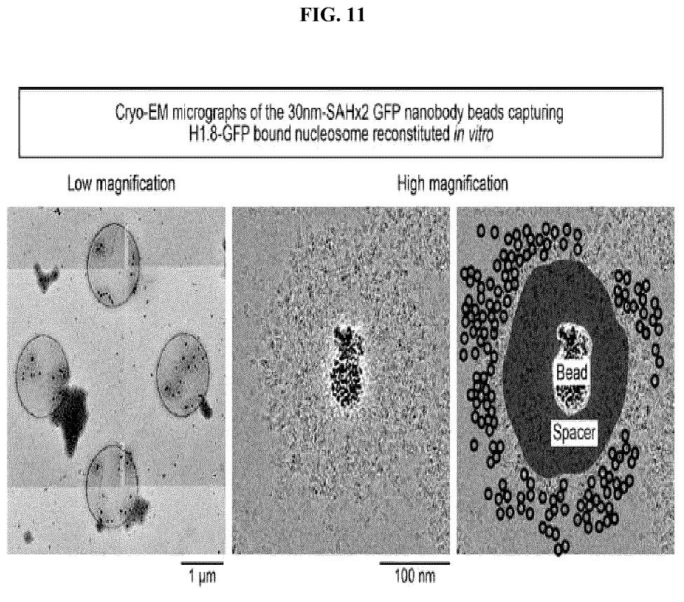

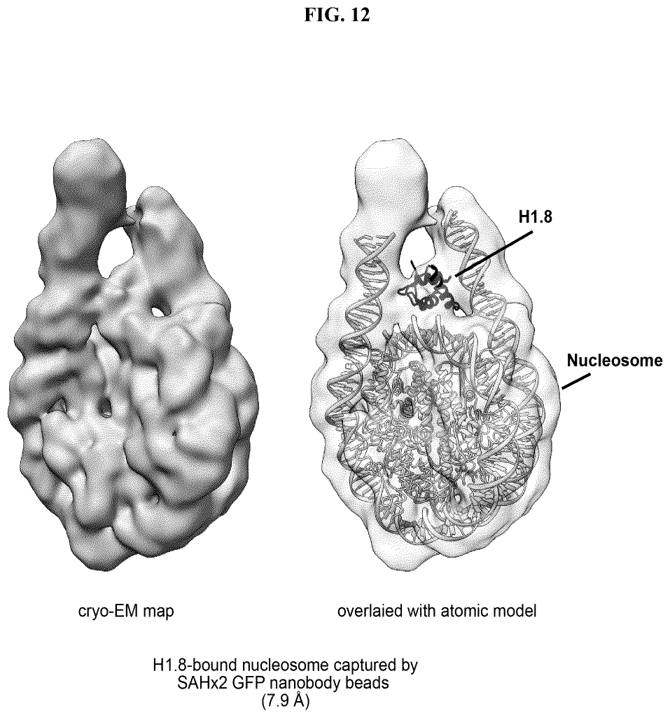

On 3HB-30 nm-SAH GFP nanobody beads, captured H1.8-GFP bound nucleosomes are accumulated too tightly, preventing it from picking many of the nucleosome-like particles on the beads. To solve this issue, biotin-3HB-SPYcatcher003 on the 3HB-30 nm-SAH GFP nanobody beads is replaced with biotin-30 nm-SAH-SPYcatcher003 to increase the length and flexibility of spacer proteins (FIG. 3: 30 nm-SAH×2 GFP nanobody beads). With the same method used for 3HB-30 nm-SAH GFP nanobody beads described in Example 5, 30 nm-SAH×2 GFP nanobody beads that captures in vitro reconstituted H1.8-GFP bound nucleosome are prepared. 4 μL of 20 pM 30 nm-SAHI×2 GFP nanobody beads that capture a maximum of 56 ng of H1.8-GFP bound nucleosomes are loaded onto a cryo-EM grid coated with monolayer graphene, concentrated by a magnetic force, and plunge frozen. With 30 nm-SAH×2 GFP nanobody beads, particles are more sparsely distributed around the beads, and more nucleosome-like particles can be picked by using Topaz from a single bead than that with 3HB-30 nm-SAH nanobody beads (FIG. 11). From 510 micrographs, the 3D structure of nucleosome that has an extra EM density around nucleosome dyad is reconstituted (FIG. 12). The resolution and quality of the structure is similar with that of 3HB-30 nm-SAH GFP nanobody beads reconstituted from 1,131 micrographs (FIG. 10). This shows that in the MagIC-cryo-EM using 30 nm-SAH×2 beads, the target particles are sparsely distributed and efficiently used for 3D structure reconstruction.

### Example 7: MagIC-Cryo-EM with Minimum Amount of In Vitro Reconstituted H1.8-GFP Bound Nucleosome Using 3HB-60 nm-SAH Nano Magnetic Beads

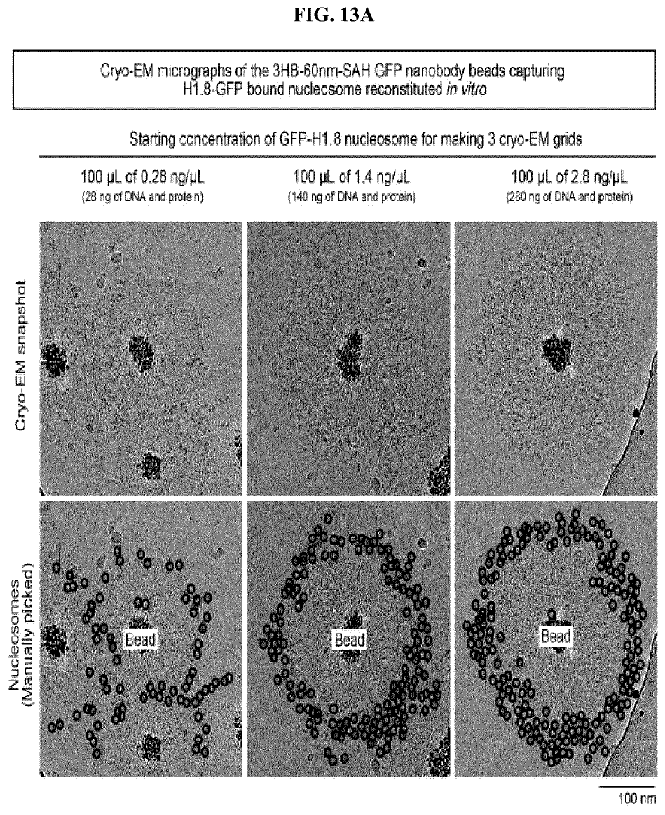

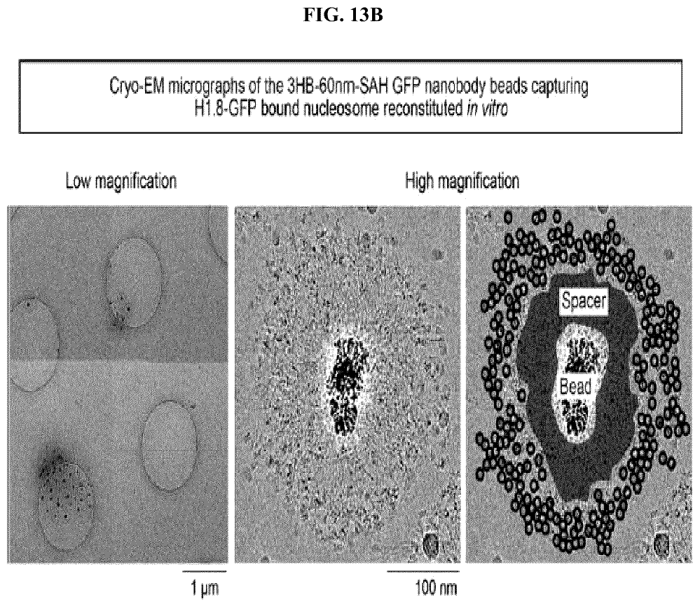

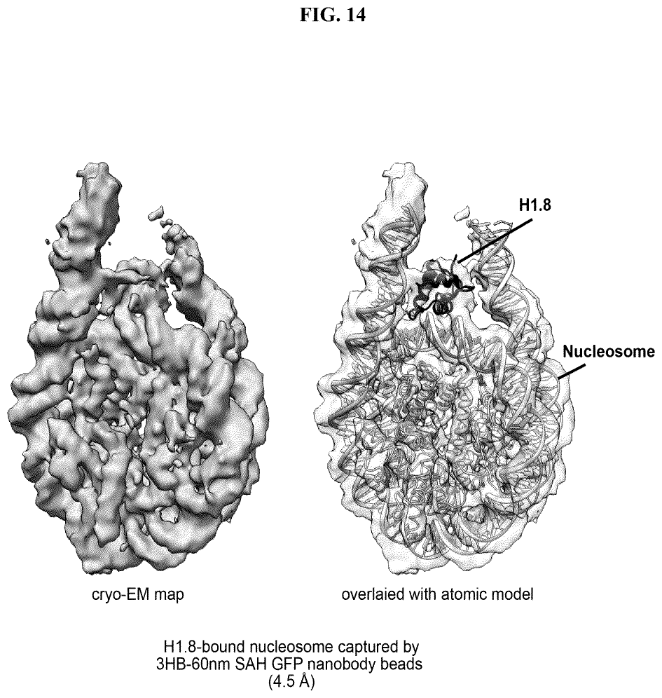

While 30 nm-SAH×2 beads provide longer space than 3HB-30 nm-SAH beads on average, there are many nucleosomes near the bead (FIG. 11). Also, the target capturing efficiency is reduced especially when the target concentration is low (data not shown). While not wanting to be bound by theory, the potential reason for these issues is that the spacer modules are kinked at the mono-SPY-tag avidin tetramer moiety between two 30 nm-SAH and the GFP nanobodies are not efficiently faced with the solvent. To solve this issue, 3HB-60 nm-SAH GFP nanobody beads are prepared (FIG. 3: 3HB-60 nm-SAH GFP nanobody beads). Also, to mimic the situation isolating low-abundant proteins, 100 μL of 0.28, 1.4, 2.8 ng DNA and protein/μL (1, 5, and 10 nM) H1.8-GFP bound nucleosome in 15% glycerol containing solution is used for MagIC-cryo-EM. In a conventional method, structural analysis starting from such a low concentration sample is nearly impossible. The removal of the glycerol and the concentration of protein are necessary, and the sample loss during dialysis by membrane bag and concentration by centrifugal filter units are significant at such a low concentration. With 0.5 fmol 3HB-60 nm-SAH GFP nanobody beads, H1.8-GFP bound nucleosomes are captured, washed, and resuspended with 15 μL buffer (theoretical beads concentration: 33 PM). 4 μL of 33 PM 3HB-60 nm-SAH GFP nanobody beads that capture a maximum of 7.4, 37.3, and 74.7 ng of H1.8-GFP bound nucleosomes are loaded onto a cryo-EM grid coated with a monolayer graphene, concentrated by a magnetic force, and plunge frozen. With using 100 μL of 0.28 ng/μL H1.8-GFP bound nucleosomes, more than 20 nucleosomes are observed around a single bead on a cryo-EM micrograph (FIG. 13A left). The numbers of the nucleosomes that can be used for structural analysis are significantly increased with using 100 μL of 1.4 ng/u L H1.8-GFP bound nucleosomes (FIG. 13A middle) while the further increase of the concentration to 100 μL of 2.8 ng/μL does not improve the numbers of the usable nucleosome on micrograph (FIG. 13A right). Therefore, the condition started from 1.4 ng/μL H1.8-GFP bound nucleosomes are used for single particle analysis (FIG. 13B). Using 493 micrographs. H1.8-GFP bound nucleosome structure is determined at 4.5 Å resolution (FIG. 14). The extra EM density around the nucleosome dyad matches with H1 atomic model. This example shows that a 100 μL solution that contains the target sample at a very low concentration (1.4 ng/μL or 5 nM) is enough for the cryo-EM structural determination using 3HB-60 nm-SAH beads.

### Example 8: MagIC-Cryo-EM of H1.8-GFP Bound Nucleosome Formed in Xenopus Egg Extract

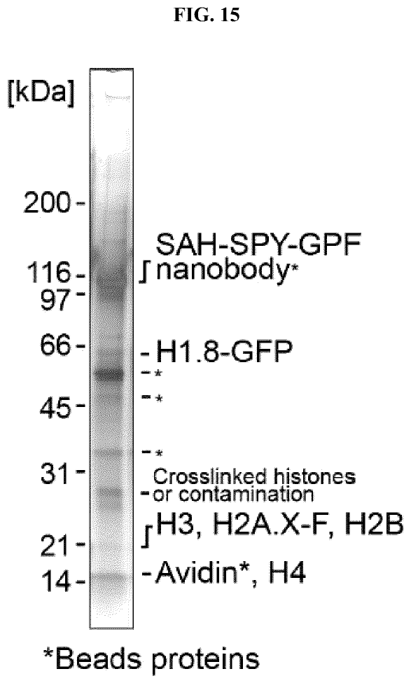

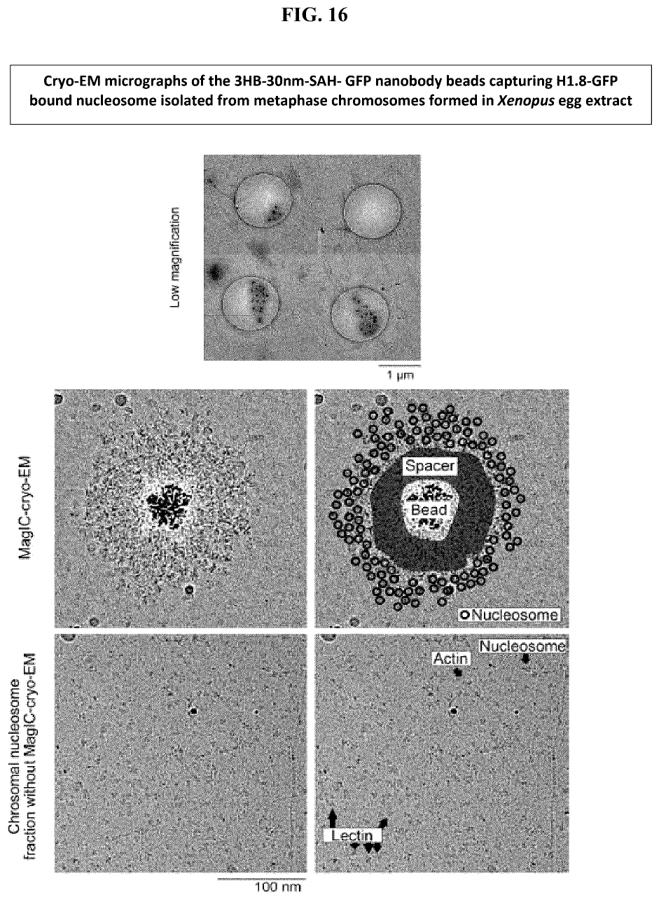

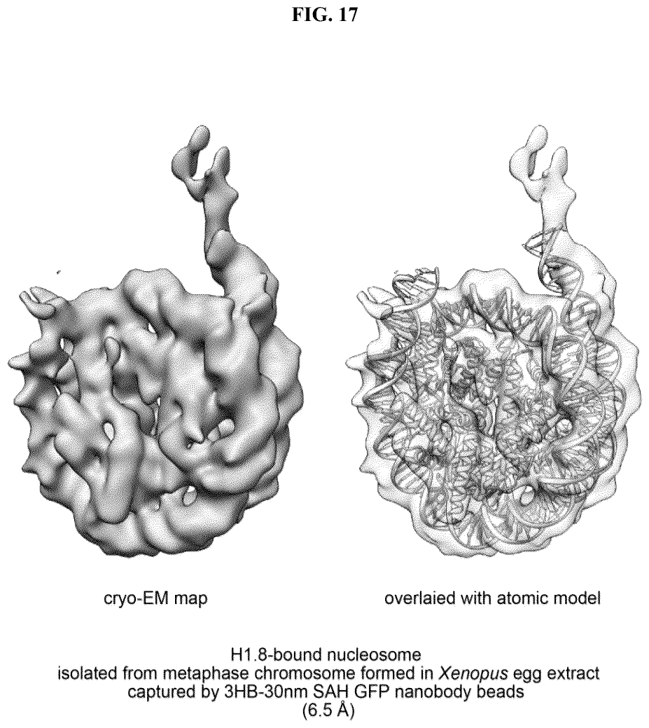

MagIC-cryo-EM of the GFP-H1.8 bound nucleosome formed in metaphase chromosomes is tested. To prepare GFP-H1.8 contained metaphase chromosomes, Xenopus laevis sperms are incubated to the GFP-H1.8 added Xenopus egg extract. Ca²⁺ ion is added to release the arrested cell cycle to interphase. After the 90 min incubation of the sperm DNA interphase egg extract, cell cycle is further progressed to metaphase by adding M phase egg extract and cyclin B truncation mutant (490). The metaphase chromosomes are crosslinked and isolated by the sucrose cushion method. The linker DNA between nucleosomes is cleaved with micrococcal nuclease (MNase), and nucleosomes are isolated by the sucrose gradient method. In this lot of preparation, MNase activity was too high, and nucleosomes lost the linker DNA (data not shown). A sucrose gradient fraction is diluted to 250 μL with PBS containing 5% glycerol and 0.01% tween-20 to reduce sucrose concentration. In the diluted sucrose gradient fraction, concentration of GFP-H1.8 is roughly 1.6 nM. Prior to mixing with nanomagnetic beads, the sample is mixed with decoy magnetic beads that are 2.8 μm large magnetic beads that bind biotin-3HB-SPYcatcher003, mono-SPYtag-avidin tetramer, and biotin-30 nm-SAH-SPYcatcher003 to remove biomolecules that are non-specifically bind to spacer module proteins. After removing the decoy beads, the GFP-H1.8 bound nucleosomes are captured by 0.5 fmol of 3HB-30 nm-SAH GFP nanobody beads, loaded onto a cryo-EM grid coated with a monolayer graphene, concentrated by a magnetic force, and plunge frozen (FIG. 15). Although the sucrose gradient fraction contains many non-nucleosome proteins (Arimura and Funabiki, 2022; Arimura et al., 2021) (e.g., Actin: long fiber, Lectin: 5 nm triangle or 20 nm three-petaled flower structures, alpha-2 macroglobulin: 30 nm four-petaled flower structure), nucleosome-like particles are specifically enriched around the beads, and no contaminated protein are found outside the beads (FIG. 16). This suggests that the 3HB-30 nm-SAH GFP nanobody beads specifically capture GFP-H1.8 bound nucleosomes. Using 905 micrographs, nucleosome structure is determined at 6.5 Å resolution (FIG. 17). Although H1.8 is not visible as cryo-EM density likely due to the too much MNase treatment that cleaves the entire linker DNA regions which are known to be necessary to stabilize the structures of H1 (Dombrowski et al., 2022), successful 3D reconstruction of the nucleosome structure indicates that MagIC-cryo-EM enables the isolation and structural determination of the low-abundant biomolecules formed in cellular environments.

### Example 9: Methods and Materials

This example describes the general methods and materials used in Examples 10 and 11.

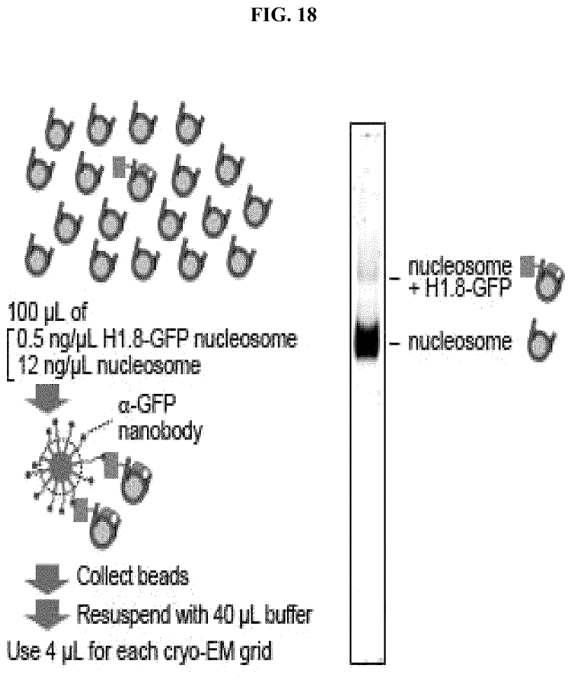

### Protein Purification (Related to Example 10 and 11 and FIGS. 18-23)

To express SPYtag-singular GFP nanobody, a plasmid pSPY-GFP nanobody was built. The plasmid has a pQE80 backbone, and the DNA sequence that encodes His₁₄-bdSUMO-His-SPY-GFP nanobody was inserted into the multiple cloning sites of the backbone. His₁₄-bdSUMO-His-SPY-GFP nanobody is expressed at 16° C. in E. coli Rosetta (DE3) by IPTG induction. The cells expressing His₁₄-bdSUMO-SPYtag-GFP nanobody are resuspended with 100 ml buffer A and are disrupted by sonication. The soluble fraction was collected with centrifugation at 20,000 rpm (46,502 rcf) at 4° C. for 30 min using 45Ti rotor in Optima L80 (Beckman Coulter) and applied to the HisTrap HP column (Cytiva). The protein was eluted from column with the step gradient of imidazole (50, 200, 400 mM) in buffer F (50 mM Tris-HCl (pH 8), 100 mM NaCl, 800 mM Imidazole, 5% Glycerol). The eluted His 14-bdSUMO-SPYtag-GFP nanobody is mixed with His₁₄-SENP1 and dialyzed against PBS containing 5% glycerol at 4° C. overnight. The dialyzed protein is applied to the HisTrap HP column (Cytiva) to remove the cleaved His₁₄-bdSUMO-tag and His₁₄-SENP1. The cleaved SPYtag-GFP-nanobody was concentrated with Amicon 10K (Millipore). The concentrated SPYtag-singular GFP nanobody was further purified through Hiload superdex75 (Cytiva) and stored at −20° C. in PBS containing 47.5% glycerol. Listed below are the DNA sequence of pQE80-His₁₄-bdSUMO-His-SPY-GFP nanobody and the amino acid sequence of SPYtag-GFP nanobody.

>pSPY-GFP nanobody
(SEQ ID NO: 22)
TTAAGGGATTTTGGTCATGACATTAACCTATAAAAATAGGCGTATCACGAGGCCCTT
TCGTCTTCACCTCGAGAAATCATAAAAAATTTATTTGCTTTGTGAGCGGATAACAAT
TATAATAGATTCAATTGTGAGCGGATAACAATTTCACACAGAATTCATTAAAGAGGA
GAAATTAACCATGAATCACAAAGTGAGCAAGCATCACCATCATTCAGGCCATCACC
ATACCGGACACCACCATCATTCAGGCAGTCATCACCATACCGGCGAGAACCTGTATT
TTCAGGGTTCAGCTGCAGGCGGTGAAGAGGATAAAAAGCCAGCCGGAGGGGAAGG
TGGTGGTGCTCATATTAACTTAAAGGTTAAAGGACAGGATGGCAACGAGGTGTTCTT
CCGCATTAAGCGCAGTACGCAACTGAAAAAACTGATGAACGCCTACTGTGACCGTC
AATCAGTTGACATGACTGCCATTGCGTTCTTATTTGACGGTCGCCGCCTGCGTGCAG
AACAGACACCAGACGAGTTAGAAATGGAAGATGGAGATGAGATCGATGCGATGTTG
CATCAGACAGGCGGTGCGCGTGGTGTCCCGCACATCGTTATGGTTGACGCGTACAAA
CGTTACAAAcatatggcccaagttcagctggttgagagtggtggtgcgctggttcagccgggtggtagtctgcgtctcagttgcgcc
gcgagcggtttcccggtgaatcgctacagtatgcgctggtaccgtcaagccccgggcaaagaacgcgaatgggttgccggtatgagtagt
gccggtgatcgcagcagctatgaagacagcgtgaaaggccgctttaccatcagccgcgatgatgcgcgcaataccgtgtacctccagatg
aacagtctgaagccagaggataccgccgtgtactactgcaacgtgaacgtgggcttcgaatattggggtcaaggcacgcaagttaccgtta
gtTAGTAAGCTTAATTAGCTGAGCTTGGACTCCTGTTGATAGATCCAGTAATGACCTC
AGAACTCCATCTGGATTTGTTCAGAACGCTCGGTTGCCGCCGGGCGTTTTTTATTGGT
GAGAATCCAAGCTAGCCATGAAAATAAACTGTCTGCTTACATAAACAGTAATACAA
GGGGTGTTATGAGCCATATTCAACGGGAAACGTCTTGCTCTAGGCCGCGATTAAATT
CCAACATGGATGCTGATTTATATGGGTATAAATGGGCTCGCGATAATGTCGGGCAAT
CAGGTGCGACAATCTATCGATTGTATGGGAAGCCCGATGCGCCAGAGTTGTTTCTGA
AACATGGCAAAGGTAGCGTTGCCAATGATGTTACAGATGAGATGGTCAGACTAAAC
TGGCTGACGGAATTTATGCCTCTTCCGACCATCAAGCATTTTATCCGTACTCCTGATG
ATGCTTGGTTACTCACGACTGCGATCCCCGGCAAAACAGCATTCCAGGTATTAGAAG
AATATCCTGATTCAGGTGAAAATATTGTTGATGCGCTGGCAGTGTTCCTGCGCCGGT
TGCATTCGATTCCTGTTTGTAATTGTCCTTTTAACAGCGATCGCGTATTTCGTCTCGC
TCAGGCGCAATCACGAATGAATAACGGTTTGGTTGATGCGAGTGATTTTGATGACGA
GCGTAATGGCTGGCCTGTTGAACAAGTCTGGAAAGAAATGCACAAACTTTTGCCATT
CTCACCGGATTCAGTCGTCACTCATGGTGATTTCTCACTTGATAACCTTATTTTTGAC
GAGGGGAAATTAATAGGTTGTATTGATGTTGGACGAGTCGGAATCGCAGACCGATA
CCAGGATCTTGCCATCCTATGGAACTGCCTCGGTGAGTTTTCTCCTTCATTACAGAAA
CGGCTTTTTCAAAAATATGGTATTGATAATCCTGATATGAATAAATTGCAGTTTCATT
TGATGCTCGATGAGTTTTTCTAAGAATTAATTCATGGGCAAATATTATACGCAAGGC
GACAAGGTGCTGATGCCGCTGGCGATTCAGGTTCATCATGCCGTTTGTGATGGCTTC
CATGTCGGCAGAATGCTTAATGAATTACAACAGTACTGCGATGAGTGGCAGGGCGG
GGCGTAATTTTTTTAAGGCAGTTATTGGTGCCCTTAAACGCCTGGGGTAATGACTCT
CTAGCTTGAGGCATCAAATAAAACGAAAGGCTCAGTCGAAAGACTGGGCCTTTCGT
TTTATCTGTTGTTTGTCGGTGAACGCTCTCCTGAGTAGGACAAATCCGCCCTCTAGAT
TACGTGCAGTCGATGATAAGCTGTCAAACATGAGAATTGTGCCTAATGAGTGAGCTA
ACTTACATTAATTGCGTTGCGCTCACTGCCCGCTTTCCAGTCGGGAAACCTGTCGTGC
CAGCTGCATTAATGAATCGGCCAACGCGCGGGGAGAGGCGGTTTGCGTATTGGGCG
CCAGGGTGGTTTTTCTTTTCACCAGTGAGACGGGCAACAGCTGATTGCCCTTCACCG
CCTGGCCCTGAGAGAGTTGCAGCAAGCGGTCCACGCTGGTTTGCCCCAGCAGGCGA
AAATCCTGTTTGATGGTGGTTAACGGCGGGATATAACATGAGCTGTCTTCGGTATCG
TCGTATCCCACTACCGAGATATCCGCACCAACGCGCAGCCCGGACTCGGTAATGGC
GCGCATTGCGCCCAGCGCCATCTGATCGTTGGCAACCAGCATCGCAGTGGGAACGA
TGCCCTCATTCAGCATTTGCATGGTTTGTTGAAAACCGGACATGGCACTCCAGTCGC
CTTCCCGTTCCGCTATCGGCTGAATTTGATTGCGAGTGAGATATTTATGCCAGCCAG
CCAGACGCAGACGCGCCGAGACAGAACTTAATGGGCCCGCTAACAGCGCGATTTGC
TGGTGACCCAATGCGACCAGATGCTCCACGCCCAGTCGCGTACCGTCTTCATGGGAG
AAAATAATACTGTTGATGGGTGTCTGGTCAGAGACATCAAGAAATAACGCCGGAAC
ATTAGTGCAGGCAGCTTCCACAGCAATGGCATCCTGGTCATCCAGCGGATAGTTAAT
GATCAGCCCACTGACGCGTTGCGCGAGAAGATTGTGCACCGCCGCTTTACAGGCTTC
GACGCCGCTTCGTTCTACCATCGACACCACCACGCTGGCACCCAGTTGATCGGCGCG
AGATTTAATCGCCGCGACAATTTGCGACGGCGCGTGCAGGGCCAGACTGGAGGTGG
CAACGCCAATCAGCAACGACTGTTTGCCCGCCAGTTGTTGTGCCACGCGGTTGGGAA
TGTAATTCAGCTCCGCCATCGCCGCTTCCACTTTTTCCCGCGTTTTCGCAGAAACGTG
GCTGGCCTGGTTCACCACGCGGGAAACGGTCTGATAAGAGACACCGGCATACTCTG
CGACATCGTATAACGTTACTGGTTTCACATTCACCACCCTGAATTGACTCTCTTCCGG
GCGCTATCATGCCATACCGCGAAAGGTTTTGCACCATTCGATGGTGTCGGAATTTCG
GGCAGCGTTGGGTCCTGGCCACGGGTGCGCATGATCTAGAGCTGCCTCGCGCGTTTC
GGTGATGACGGTGAAAACCTCTGACACATGCAGCTCCCGGAGACGGTCACAGCTTG
TCTGTAAGCGGATGCCGGGAGCAGACAAGCCCGTCAGGGCGCGTCAGCGGGTGTTG
GCGGGTGTCGGGGCGCAGCCATGACCCAGTCACGTAGCGATAGCGGAGTGTATACT
GGCTTAACTATGCGGCATCAGAGCAGATTGTACTGAGAGTGCACCATATGCGGTGTG
AAATACCGCACAGATGCGTAAGGAGAAAATACCGCATCAGGCGCTCTTCCGCTTCC
TCGCTCACTGACTCGCTGCGCTCGGTCGTTCGGCTGCGGCGAGCGGTATCAGCTCAC
TCAAAGGCGGTAATACGGTTATCCACAGAATCAGGGGATAACGCAGGAAAGAACAT
GTGAGCAAAAGGCCAGCAAAAGGCCAGGAACCGTAAAAAGGCCGCGTTGCTGGCG
TTTTTCCATAGGCTCCGCCCCCCTGACGAGCATCACAAAAATCGACGCTCAAGTCAG
AGGTGGCGAAACCCGACAGGACTATAAAGATACCAGGCGTTTCCCCCTGGAAGCTC
CCTCGTGCGCTCTCCTGTTCCGACCCTGCCGCTTACCGGATACCTGTCCGCCTTTCTC
CCTTCGGGAAGCGTGGCGCTTTCTCATAGCTCACGCTGTAGGTATCTCAGTTCGGTG
TAGGTCGTTCGCTCCAAGCTGGGCTGTGTGCACGAACCCCCCGTTCAGCCCGACCGC
TGCGCCTTATCCGGTAACTATCGTCTTGAGTCCAACCCGGTAAGACACGACTTATCG
CCACTGGCAGCAGCCACTGGTAACAGGATTAGCAGAGCGAGGTATGTAGGCGGTGC
TACAGAGTTCTTGAAGTGGTGGCCTAACTACGGCTACACTAGAAGGACAGTATTTGG
TATCTGCGCTCTGCTGAAGCCAGTTACCTTCGGAAAAAGAGTTGGTAGCTCTTGATC
CGGCAAACAAACCACCGCTGGTAGCGGTGGTTTTTTTGTTTGCAAGCAGCAGATTAC
GCGCAGAAAAAAAGGATCTCAAGAAGATCCTTTGATCTTTTCTACGGGGTCTGACGC
TCAGTGGAACGAAAACTCACG
>His₁₄-bdSUMO-His-SPY-GFP nanobody
(SEQ ID NO: 23)
ARGVPHIVMVDAYKRYKHMAQVQLVESGGALVQPGGSLRLSCAASGFPVNRYSMRW
YRQAPGKEREWVAGMSSAGDRSSYEDSVKGRFTISRDDARNTVYLQMNSLKPEDTAV
YYCNVNVGFEYWGQGTQVTVS

### Magnetic Concentration of Nano-Magnetic Beads on Cryo-EM Grid (Related to Example 10-11 and FIGS. 18-23)

A glow discharged Quantifoil gold R1.2/1.3 grid (Quantifoil) coated with monolayer graphene (Han et al., 2020) was anchored with a pair of sharp non-magnetic tweezers (SubAngstrom) that can be attached to the Vitrobot Mark IV (FEI). After applying 4 μL of nano-magnetic beads solution to the grid, the grid was incubated on the 40×20 mm N52 neodymium disc magnets for 5 min in a high humidity chamber. After completion of the magnetic bead capture, the tweezers anchoring the grid are attached to the Vitrobot Mark IV (FEI), and the grid vitrified by 2 scc blotting time at room temperature under 100% humidity.

MagIC-Cryo-EM of H1.8-GFP Bound Nucleosome Isolated from a Low-Purity Sample (Related to Example 10 and FIGS. 18-20)

0.5 fmol of GFP-singular nanobodies conjugated 3HB-60 nm-SAH magnetic beads are mixed with 1.7 nM (0.5 ng/μL) H1.8-GFP bound nucleosome and 53 nM (12 ng/μL) nucleosome in 100 μL of buffer SG (15 mM HEPES-KOH (pH 7.4). 50 mM KCl 12% sucrose, 1×LPC, 10 mM Sodium Butyrate, 10 mM beta-glycerophosphate, 1 mM EGTA) containing about 17% sucrose and incubated at 4° C. for 10 hours. To wash the beads, the beads are collected with centrifugation at 13,894 rpm (16.000 rcf) at 4° C. for 20 min using SX241.5 rotor in Allegron X-30R (Beckman Coulter) and resuspended with 200 μL of EM buffer C (10 mM HEPES-KOH (pH 7.4), 30 mM KCl, 1 mM EGTA, 10 ng/μL leupeptin, 10 ng/μL pepstatin, 10 ng/μL chymostatin, 1 mM sodium butyrate, 1 mM beta-glycerophosphate. 1.2% trehalose, and 0.12% 1,6-hexanediol). This washing step is repeated twice, and the beads are resuspended with about 40 μL of EM buffer C (theoretical beads concentration: 12.5 pM).

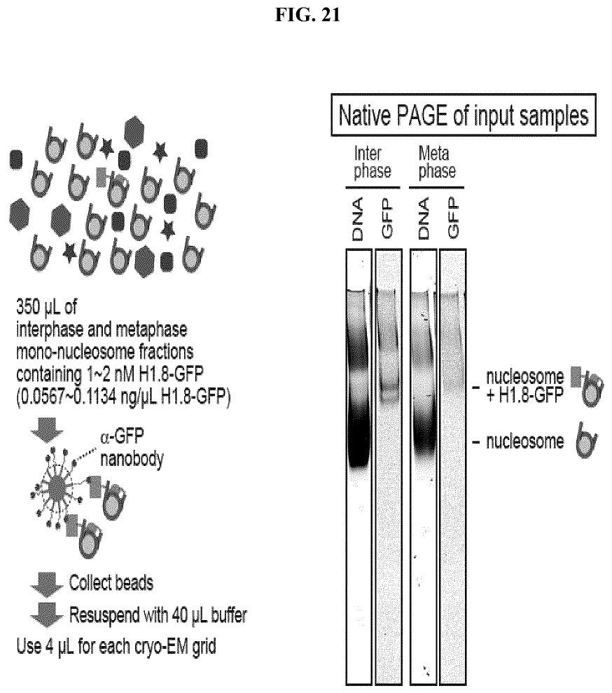

Nucleosome Isolation from Xenopus Egg Extracts Chromosomes (Related to Example 11 and FIGS. 21-23)

Nucleosomes were isolated from Xenopus egg extracts chromosomes with the previously described method (Arimura et al . . . 2021). The cytostatic factor (CSF) metaphase-arrested Xenopus laevis egg extracts are prepared with the method described previously (Murray, 1991). To prevent spontaneous cycling of egg extracts, 0.1 mg/ml cycloheximide is added to the CSF extract. H1.8-GFP are added to the CSF extract at a final concentration of 650 nM, which is an equal to the concentration with endogenous H1.8 (Wühr et al., 2014). For interphase chromosome preparation, Xenopus laevis sperm nuclei (final concentration 2000/μl) are added to 5 ml of CSF extracts, which are incubated for 90 min at 20° C. after adding 0.3 mM CaCl₂), which releases CSF extracts into interphase. For the metaphase sperm chromosome preparation, cyclin B Δ90 (final concentration 24 μg/ml) and 1 ml fresh CSF extract are added to 2 ml of the extract containing interphase sperm nuclei prepared with the method described above. The extracts are incubated for 60 min at 20° C. during which each tube is gently mixed every 10 min. To crosslink the Xenopus egg extracts chromosomes, nine times the volume of ice-cold buffer XL (80 mM PIPES-KOH [pH 6.8], 15 mM NaCl. 60 mM KCl, 30% glycerol. 1 mM EGTA, 1 mM MgCl₂, 10 mM beta-glycerophosphate, 10 mM sodium butyrate, 2.67% formaldehyde) is added to the interphase or metaphase extract with chromosomes, which is further incubated for 60 min on ice. These fixed chromosomes are layered on 3 ml of fresh buffer SC (80 mM HEPES-KOH [pH 7.4], 15 mM NaCl. 60 mM KCl, 1.17 M sucrose, 50 mM glycine, 0.15 mM spermidine, 0.5 mM spermine, 1.25× complete EDTA-free Protease Inhibitor Cocktail (Roche). 10 mM beta-glycerophosphate, 10 mM sodium butyrate, 1 mM EGTA. 1 mM MgCl₂) in 50 ml centrifuge tubes (Falcon. #352070) and spun at 3.300 (2.647 rcf) rpm at 4° C. for 40 min using JS 5.3 rotor in Avanti J-26S (Beckman Coulter). Pellets containing fixed chromosomes are resuspended with 10 ml of buffer SC, layered on 3 ml of fresh buffer SC in 14 ml centrifuge tubes (Falcon, #352059), and spun at 3.300 (2.647 rcf) rpm at 4° C. for 40 min using JS 5.3 rotor in Avanti J-26S (Beckman Coulter). The chromosomes are collected from the bottom of the centrifuge tube and resuspended with buffer SC. Chromosomes are pelleted by centrifugation at 5,492 rpm (2.500 rcf) using SX241.5 rotor in Allegron X-30R (Beckman Coulter). The chromosome pellets are resuspended with 200 μL of buffer SC. To digest chromatin, 0.6 and 0.3 U/μL of MNase (Worthington Biochemical Corporation) is added to interphase and metaphase chromosome, respectively. Then, CaCl₂) are added to 7.4 mM, and the mixture is incubated at 4° C. for 4 h. MNase reaction is stopped by adding 100 μL MNase stop buffer B (80 mM PIPES-KOH (pH 6.8). 15 mM NaCl, 60 mM KCl, 30% Glycerol, 20 mM EGTA, 1 mM MgCl₂, 10 mM beta-glycerophosphate. 10 mM Sodium Butyrate. 3.00% formaldehyde). The mixtures are incubated on ice for 1 h and then diluted with 700 μL of quench buffer (30 mM HEPES-KOH (pH 7.4), 150 mM KCl, 1 mM EGTA 1×LPC. 10 mM Sodium Butyrate. 10 mM beta-glycerophosphate, 400 mM glycine, 1 mM MgCl₂, 5 mM DTT). The soluble fractions released by MNase are isolated by taking supernatants after centrifugation at 13,894 rpm (16,000 rcf) at 4° C. for 30 min using SX241.5 rotor in Allegron X-30R (Beckman Coulter). The supernatants are collected and layered onto the 10-22% linear sucrose gradient solution with buffer SG B (15 mM HEPES-KOH [pH 7.4], 50 mM KCl, 10-22% sucrose, 10 μg/ml leupeptin, 10 μg/ml pepstatin, 10 μg/ml chymostatin, 10 mM sodium butyrate. 10 mM beta-glycerophosphate. 1 mM EGTA. 20 mM glycine) and spun at 32,000 rpm (max 124,436 rcf) and 4° C. for 13 h using SW55Ti rotor in Optima L80 (Beckman Coulter). The samples are fractionated from the top of the sucrose gradient. The concentration of the H1.8 in each fraction is determined by western blot. 15 μL each of sucrose gradient fraction is incubated at 95° C. under the existence of 1% Sodium dodecyl sulfate (SDS) applied for SDS-PAGE with 4-20% gradient SDS-PAGE gel (Bio-rad). The proteins are transferred to the nitrocellulose membrane (Cytiva) from the SDS-PAGE gel using TE42 Tank Blotting Units (Hoefer) at 15 V. 4° C. for 4 h. As primally antibodies. 1 μg/ml of mouse monoclonal Anti-GFP Antibody sc-9996 (Santa Cruz Biotechnology) and as secondary antibodies, IR Dye 800CW goat anti-mouse IgG (Li-Cor 926-32210; 1:15,000) are used. The images are taken with Odyssey Infrared Imaging System (Li-Cor). The existence of the H1.8-GFP bound nucleosomes is confirmed by native PAGE. 15 μL each of sucrose gradient fraction is applied for 6%×0.5 TEB native PAGE gel. The DNA is stained with SYTO-60. The images of SYTO-60 signal and GFP signal are taken with Odyssey Infrared Imaging System (Li-Cor).

MagIC-Cryo-EM of H1.8-GFP Bound Nucleosome Isolated from Xenopus Egg Extract Using Nano-Magnetic Beads (Related to Example 11 and FIGS. 21-23)

10 fmol of GFP-singular nanobodies conjugated 3HB-60 nm-SAH magnetic beads are mixed with the 350 μL of interphase or metaphase sucrose gradient fraction that contains H1.8-GFP bound nucleosome and incubated at 4° C. for 15 h. To wash the beads, the beads are collected with centrifugation at 13,894 rpm (16,000 rcf) at 4° C. for 20 min using SX241.5 rotor in Allegron X-30R (Beckman Coulter) and resuspended with 200 μL of EM buffer C. This washing step is repeated twice, and the beads are resuspended with about 40 μL of EM buffer C (theoretical beads concentration: 25 pM).

### Example 10: MagIC-Cryo-EM of H1.8-GFP Bound Nucleosome Isolated from a Low-Purity Sample (Related to FIGS. 18-20)

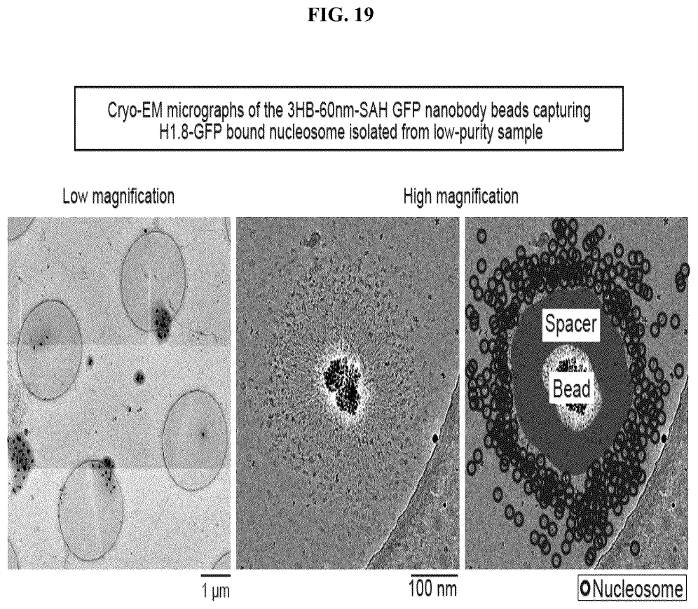

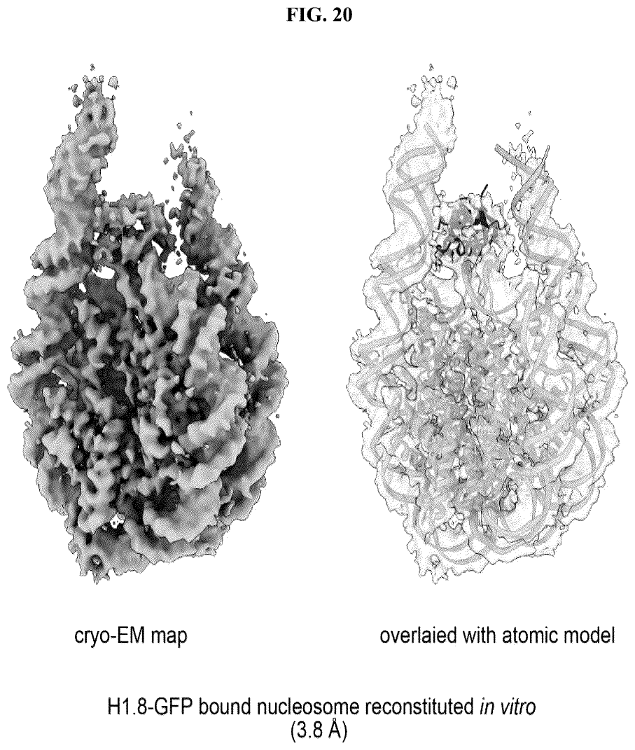

In a realistic situation, large amounts of unwanted protein complexes are contained in the sample. To simulate such a situation, 1.7 nM (0.5 ng/μL) H1.8-GFP bound nucleosome and 53 nM (12 ng/μL) nucleosome are mixed and are used for MagIC-cryo-EM. Instead of the SPYtag-tandem GFP nanobody, the SPYtag-singular GFP nanobody is used for this experiment. While tandem GFP nanobody has a high affinity to GFP, it also induces aggregation of the nano magnetic beads. H1.8-GFP bound nucleosomes are captured, washed, and resuspended with 40 μL buffer (theoretical beads concentration: 12.5 pM). 4 μL, of 12.5 pM 3HB-60 nm-SAH GFP nanobody beads that capture H1.8-GFP bound nucleosomes are loaded onto a cryo-EM grid coated with monolayer graphene, concentrated by a magnetic force, and plunge frozen (FIG. 19). Using 1890 micrographs, H1.8-GFP bound nucleosome structure is determined at 3.8 Å resolution (FIG. 20). The extra EM density around the nucleosome dyad matches with the H1 atomic model. This example shows that the MagIC-cryo-EM is capable of isolating the target complex from low-purity input that contains the target complexes at low concentrations (1.7 nM, 0.5 ng/μL).

### Example 11: MagIC-Cryo-EM of H1.8-GFP Bound Nucleosome Formed in Interphase and Metaphase Chromosomes in Xenopus Egg Extract (Related to FIGS. 21-23)

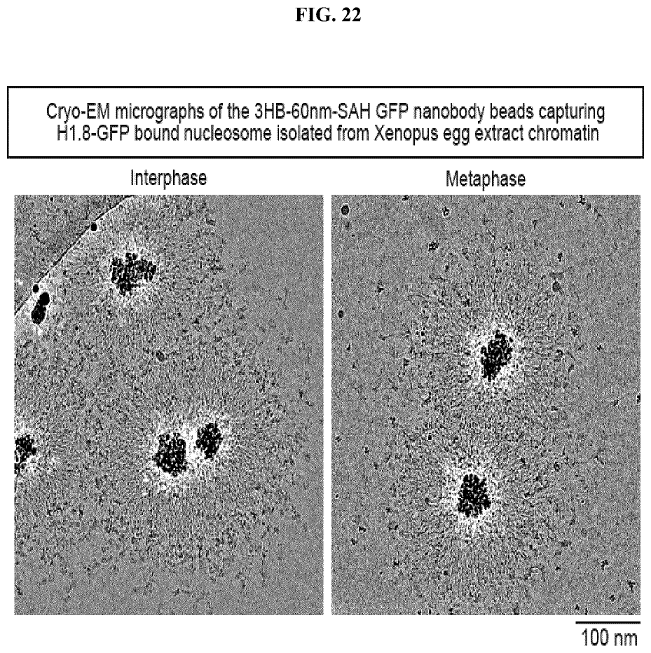

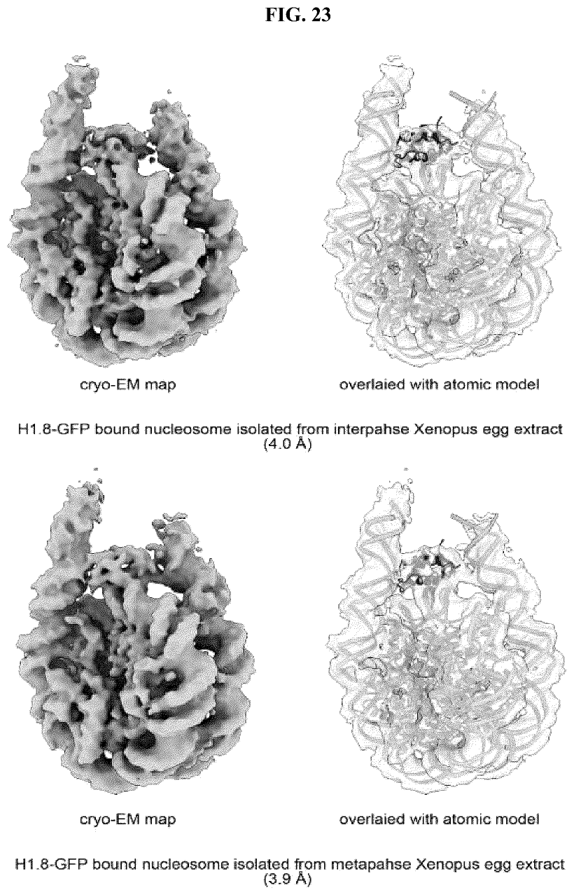

To examine if the MagIC-cryo-EM can isolate a target complex from a crude cellular fraction. H1.8-GFP bound nucleosomes formed in interphase and metaphase chromosomes in Xenopus egg extract are isolated (FIG. 21). Using 350 μL of interphase and metaphase chromosome fractions that contain 1˜2 nM H1.8-GFP and 10 fmol of GFP-singular nanobodies conjugated with 3HB-60 nm-SAH magnetic beads. H1.8-GFP bound nucleosomes are captured, washed, and resuspended in 40 μL EM buffer C (theoretical beads concentration: 25 PM). 4 μL of 25 pM 3HB-60 nm-SAH GFP nanobody beads that capture H1.8-GFP bound nucleosomes are loaded onto a cryo-EM grid coated with monolayer graphene, concentrated by a magnetic force, and plunge frozen. 677 and 965 micrographs are collected for interphase and metaphase, respectively (FIG. 22). Using these micrographs, H1.8-GFP bound nucleosome structure is determined at 4.0 and 3.9 Å resolution for interphase and metaphase, respectively. (FIG. 23). The extra EM density around the nucleosome dyad matches with the H1 atomic model. This example shows that the MagIC-cryo-EM is capable of isolating the target complex from a crude cellular fraction that contains the target complexes at low concentrations (1˜2 nM).

The publications described in the present disclosure, including those listed below, are incorporated by reference herein.

### OTHER PUBLICATIONS

Arimura, Y., and Funabiki, H. (2022). Structural Mechanics of the Alpha-2-Macroglobulin Transformation. J. Mol. Biol. 434, 167413.
Arimura. Y., Tachiwana, H., Oda, T., Sato, M., and Kurumizaka, H. (2012). Structural analysis of the hexasome, lacking one histone H2A/H2B dimer from the conventional nucleosome. Biochemistry 51.
Arimura, Y., Shih, R. M., Froom, R., and Funabiki, H. (2021). Structural features of nucleosomes in interphase and metaphase chromosomes. Mol. Cell 87, 4377-4397.
Bepler, T., Morin, A., Rapp, M., Brasch. J., Shapiro, L., Noble, A. J., and Berger. B. (2019). Positive-unlabeled convolutional neural networks for particle picking in cryo-electron micrographs. Nat. Methods 16, 1153-1160.
Bonnafous, P., Perrault, M., Le Bihan, O., Bartosch, B., Lavillette, D., Penin, F., Lambert, O., and Pécheur, E. I. (2010). Characterization of hepatitis C virus pseudoparticles by cryo-transmission electron microscopy using functionalized magnetic nanobeads. J. Gen. Virol. 91, 1919-1930.
Brizzard. B. (2008). Epitope tagging. Biotechniques 44, 693-695.
Carragher, B., Cheng. Y., Frost, A., Glaeser. R. M., Lander, G. C., Nogales, E., and Wang, H. W. (2019). Current outcomes when optimizing ‘standard’ sample preparation for single-particle cryo-EM. J. Microsc. 276, 39-45.
D'Imprima, E., Floris, D., Joppe, M., Sánchez, R., Grininger. M., and Kühlbrandt, W. (2019). Protein denaturation at the air-water interface and how to prevent it. Elife 8, 1-18.
Dombrowski. M., Engcholm, M., Dienemann, C., Dodonova, S., and Cramer, P. (2022). Histone H1 binding to nucleosome arrays depends on linker DNA length and trajectory. Nat. Struct. Mol. Biol. 29, 493-501.
Frey, S., and Görlich, D. (2014). A new set of highly efficient, tag-cleaving proteases for purifying recombinant proteins. J. Chromatogr. A 1337, 95-105.
Fujita. T., and Fujii, H. (2019). Purification of specific DNA species using the CRISPR system. Biol. Methods Protoc. 4, 1-6.
Gibson, D. G., Young, L., Chuang, R. Y., Venter, J. C., Hutchison, C. A., and Smith, H. O. (2009). Enzymatic assembly of DNA molecules up to several hundred kilobases. Nat. Methods 6, 343-345.
Guse. A., Fuller, C. J., and Straight, A. F. (2012). A cell-free system for functional centromere and kinetochore assembly. Nat. Protoc. 7, 1847-1869.
Han, Y., Fan, X., Wang. H., Zhao, F., Tully, C. G., Kong, J., Yao, N., and Yan, N. (2020). High-yield monolayer graphene grids for near-atomic resolution cryoelectron microscopy. Proc. Natl. Acad. Sci. U.S.A 117, 1009-1014.
Howarth, M., Chinnapen, D. J. F., Gerrow, K., Dorrestein. P. C., Grandy, M. R., Kelleher, N. L., El-Husseini, A., and Ting, A. Y. (2006). A monovalent streptavidin with a single femtomolar biotin binding site. Nat. Methods 3, 267-273.
Huang, P. S., Oberdorfer, G., Xu, C., Pei, X. Y., Nannenga, B. L., Rogers, J. M., DiMaio, F., Gonen. T., Luisi. B., and Baker, D. (2014). High thermodynamic stability of parametrically designed helical bundles. Science 346, 481-485.
Karkare. S., and Bhatnagar, D. (2006). Promising nucleic acid analogs and mimics: Characteristic features and applications of PNA, LNA, and morpholino. Appl. Microbiol. Biotechnol. 71, 575-586.
Keeble. A. H., Turkki, P., Stokes. S., Anuar, I. N. A. K., Rahikainen, R., Hytönen, V. P., and Howarth, M. (2019). Approaching infinite affinity through engineering of peptide-protein interaction. Proc. Natl. Acad. Sci. U.S.A 116, 26523-26533.
Lowary, P. T., and Widom, J. (1998). New DNA sequence rules for high affinity binding to histone octamer and sequence-directed nucleosome positioning. J. Mol. Biol. 276, 19-42. Murray, A. W. (1991). Cell cycle extracts. Methods Cell Biol. 36, 581-605.
Pina, A. S., Batalha, I. L., and Roque, A. C. A. (2014). Affinity Tags in Protein Purification and Peptide Enrichment: An Overview BT-Protein Downstream Processing: Design, Development and Application of High and Low-Resolution Methods. N. E. Labrou, cd. (Totowa, NJ: Humana Press). pp. 147-168.
Punjani, A., Rubinstein, J. L., Fleet. D. J., and Brubaker. M. A. (2017). CryoSPARC: Algorithms for rapid unsupervised cryo-EM structure determination. Nat. Methods 14, 290-296. Sivaramakrishnan. S., and Spudich, J. A. (2011). Systematic control of protein interaction using a modular ER/K α-helix linker. Proc. Natl. Acad. Sci. U.S.A 108, 20467 20472.
Wages. J. M., Wages, G. M., Matthews. P., Weller, D., and Summerton, J. (1997). Affinity purification of RNA: Sequence-specific capture by nonionic morpholino probes. Biotechniques 23, 1116-1121.
Wühr, M., Freeman, R. M., Presler, M., Horb, M. E., Peshkin. L., Gygi, S. P., and Kirschner, M. W. (2014). Deep proteomics of the Xenopus laevis egg using an mRNA-derived reference database. Curr. Biol. 24, 1467-1475.
Zakeri, B., Fierer, J. O., Celik. E., Chittock, E. C., Schwarz-Linek, U., Moy, V. T., and Howarth, M. (2012). Peptide tag forming a rapid covalent bond to a protein, through engineering a bacterial adhesin. Proc. Natl. Acad. Sci. U.S.A 109.
Zhang, Z., Wang. Y., Ding. Y., and Hattori, M. (2020). Structure-based engineering of anti-GFP nanobody tandems as ultra-high-affinity reagents for purification. Sci. Rep. 10, 1-10.
Zierhut. C., Jenness, C., Kimura, H., and Funabiki, H. (2014). Nucleosomal regulation of chromatin composition and nuclear assembly revealed by histone depletion. Nat. Struct. Mol. Biol. 27, 617-625.

This invention may be embodied in other forms or carried out in other ways without departing from the spirit or essential characteristics thereof. The present disclosure is therefore to be considered as in all aspects illustrated and not restrictive, the scope of the invention being indicated by the appended Claims, and all changes which come within the meaning and range of equivalency are intended to be embraced therein.

Various references are cited throughout this Specification, each of which is incorporated herein by reference in its entirety.

1. Magnetic particles that capture biological target molecules for cryo-electron microscope imaging comprising:
paramagnetic beads;
at least two spacer modules extending from a periphery of said paramagnetic beads comprising a first spacer module and a second spacer module, said first spacer module binding said paramagnetic beads and said second spacer module being located outwardly of said first spacer module and binding said first spacer module, said first spacer module comprising first spacer proteins and said second spacer module comprising second spacer proteins;
a capture module linked to an outer location of said at least two spacer modules, said capture module comprising one or more capture proteins that are adapted to capture biological target molecules by having affinity to the biological target molecules,
wherein said at least two spacer modules are arranged so that a combined length of said at least two spacer modules locates said capture module a distance that is spaced from said paramagnetic beads.

2. The magnetic particles of claim 1 wherein:
(a) said paramagnetic beads are not more than 100 nm in size, not more than 80 nm in size, not more than 60 nm in size, or not more than 50 nm in size;
(b) said first spacer proteins are type I spacer proteins and said second spacer proteins are type II spacer proteins, said first spacer proteins and said second spacer proteins being linked end to end;
(c) said first spacer proteins together with said second spacer proteins form rays extending outwardly from said paramagnetic beads;
(d) said type I spacer proteins comprise one or more monomeric triple helical bundle proteins; and/or
(e) said type II spacer proteins comprise one or more single alpha helix proteins.

3. (canceled)

4. (canceled)

5. (canceled)

6. (canceled)

7. (canceled)

8. The magnetic particles of claim 1 comprising:
(a) streptavidin that binds the paramagnetic beads, wherein said first spacer proteins comprise biotinylated monomeric 3HB, said 3HB being triple helical bundle proteins, said streptavidin binding biotin of said biotinylated 3HB proteins, said second spacer proteins comprising biotinylated SAH proteins, said SAH being single alpha helix proteins, and a mono-SPY-tagged avidin tetramer linked to an outer end portion of said biotinylated 3HB proteins and binding biotin of multiple of said biotinylated SAH proteins; or
(b) streptavidin that binds the paramagnetic beads, wherein said first spacer proteins and said second spacer proteins comprise biotinylated SAH proteins, said streptavidin binding biotin of said biotinylated SAH proteins of said first spacer module, and a mono-SPY-tagged avidin tetramer linked to an outer end portion of said biotinylated SAH proteins of said first spacer module and binding biotin of multiple of said biotinylated SAH proteins of said second spacer module.

9. The magnetic particles of claim 8 wherein:
(a) said mono-SPY-tagged avidin tetramer is linked to an outer end portion of said biotinylated 3HB proteins by bonding between SPYcatcher003 and SPYtag003moieties, said SPYcatcher003 moiety being located on an end portion of said biotinylated 3HB proteins and said SPYtag003 moiety being located on said mono-SPY-tagged avidin tetramer; or
(b) said mono-SPY-tagged avidin tetramer is linked to an outer end portion of said biotinylated SAH proteins of said first spacer module by bonding between SPYcatcher003 and SPYtag003 moieties, said SPYcatcher003 moiety being located on an end portion of said biotinylated single alpha helix proteins of said first spacer module and said SPYtag003 moiety being located on said mono-SPY-tagged avidin tetramer.

10. (canceled)

11. (canceled)

12. (canceled)

13. The magnetic particles of claim 1, wherein said capture module has affinity to at least one of the biological target molecules selected from at least one of a protein or a tag of the protein, RNA or DNA, and portions or fragments thereof, and combinations thereof.

14. The magnetic particles of claim 1 wherein said paramagnetic beads are superparamagnetic.

15. The magnetic particles of claim 1 wherein:
(a) said capture module comprises an antibody, antibody fragment, or antigen-binding portion thereof;
(b) said capture module comprises at least one of Protein A, Strep-Tactin, calmodulin binding protein, single stranded DNA, double stranded DNA, single stranded RNA, double stranded RNA, SPYcatcher, streptavidin, streptavidin that binds NTA-biotin, streptavidin that binds biotinylated glutathione, streptavidin that binds Tris-NTA-biotin, streptavidin that binds Halotag biotin ligand, streptavidin that binds chitosan-biotin, or streptavidin that binds SNAP-biotin; or
(c) said capture module comprises at least one of nuclease-dead CRISPR-associated protein 9 (dCas9) attached with single guide RNA (sgRNA), zinc-finger proteins (ZFPs), transcription activator-like (TAL) proteins, PNA, LNA, single stranded DNA, single stranded RNA, or morpholino.

16. (canceled)

17. (canceled)

18. The magnetic particles of claim 15 wherein said capture module comprises at least one of a nanobody, scFv or Fab.

19. (canceled)

20. The magnetic particles of claim 18 wherein:
(a) the at least one nanobody, scFv or Fab has affinity for a histone or a tag of said histone; or
(b) the at least one nanobody, scFv or Fab has affinity for a protein or a tag of said protein, and said protein binds to DNA or is associated with at least one other protein that bind to DNA.

21. (canceled)

22. The magnetic particles of claim 1 wherein the distance that is spaced from said paramagnetic bead is at least about 30 nm, at least about 40 nm, at least about 50 nm, at least about 60 nm, at least about 70 nm or at least about 80 nm.

23. A method of using cryo-electron microcopy to image a biological target molecule, comprising:
providing said magnetic particles of claim 1;
mixing said magnetic particles in a liquid with biological target molecules to capture the biological target molecules, said capture proteins having affinity to the biological target molecules;
applying a liquid comprising said magnetic particles with the captured biological target molecules onto an electron microscope grid;
concentrating said biological target molecules in said liquid on said electron microscope grid by applying a magnetic field to said magnetic particles with the captured biological target molecules;
removing a portion of the liquid from said electron microscope grid;
applying cryogenic conditions so as to vitrify the magnetic particles and the captured biological target molecules on the electron microscope grid; and
conducting electron microscope imaging of the biological target molecules.

24. The method of claim 23 comprising applying a sheet of material onto said electron microscope grid, and applying the liquid including said magnetic particles with the captured target molecules onto said sheet of material on said electron microscope grid.

25. The method of claim 24 wherein said sheet of material comprises graphene.

26. The method of claim 23 wherein said electron microscope imaging enables single particle analysis of the biological target molecules.

27. (canceled)

28. (canceled)

29. (canceled)

30. (canceled)

31. (canceled)

32. The method of claim 23 wherein said capture mode comprises at least one of a nanobody, scFv or Fab has affinity for a linker histone including a tag, and said linker histone binds a nucleosome, capturing said linker histone and conducting electron microscope imaging of at least one of said nucleosome and said captured linker histone as the biological target molecules.

33. The method of claim 23 comprising: capturing in vitro reconstituted recombinant histone-bound nucleosomes as the biological target molecules; capturing recombinant histone-bound nucleosomes as the biological target molecules, which have been isolated from chromosomes in a cellular environment; capturing the biological target molecules selected from at least one of a protein, RNA or DNA, and portions or fragments thereof, and combinations thereof, and conducting electron microscope imaging thereof; or capturing H1.8-GFP bound nucleosome as the biological target molecules.

34. (canceled)

35. The method of claim 23 wherein said capture module has affinity to a protein or a tag of the protein that forms a part of the biological target molecules, and wherein said tag is at least one of a GFP tag, Myc tag, HA tag. V5-tag, CD tag or FLAG tag, and combinations thereof, or said tag is a peptide and protein affinity tag including at least one of SPYtag, CBP-tag, GST-tag, poly His-tag or SNAP-tag, CDB tag, Halo tag, Avitag. S-tag, or Strep-tag and combinations thereof.

36. (canceled)

37. (canceled)

38. The method of claim 23 wherein the at least one nanobody, scFv or Fab bas affinity for a protein or a tag thereof, and said protein binds DNA or is associated with at least one other protein that binds to DNA, capturing said protein and conducting electron microscope imaging of said protein as one of the biological target molecules.

39. (canceled)

40. (canceled)

41. The method of claim 23 comprising capturing said biological target molecules present in a solution in a concentration of not more than I nM, not more than 1.6 nM, not more than 5 nM, not more than 10 nM, not more than 34 nM, or not more than 100 nM.

42. A method of the making magnetic particles for capturing biological target molecules for cryo-electron microscope imaging of claim 1 comprising:
a) providing paramagnetic beads conjugated with streptavidin;
b) adding biotin-3HB-SPYcatcher003 proteins or biotin-SAH-SPYcatcher003 proteins to said paramagnetic beads conjugated with streptavidin, wherein said 3HB is a three helix bundle and said SAH is a single alpha helix, wherein said biotin of said biotin-3HB-SPYcatcher003 proteins binds said streptavidin or said biotin of said biotin-SAH-SPYcatcher003 proteins binds said streptavidin, attaching a first spacer module;
c) adding mono-SPY-tagged avidin tetramer proteins to said first spacer module, wherein a SPYtag003moiety of said mono-SPY-tagged avidin tetramer proteins bonds the SPYcatcher003 moiety of said biotin-3HB-SPYcatcher003 proteins, or wherein a SPYtag003moiety of said mono-SPY-tagged avidin tetramer proteins bonds the SPYcatcher003 moiety of said biotin-SAH-SPYcatcher003 proteins; and
d) adding biotinylated second spacer proteins comprising SAH and SPYcatcher003 moieties to said first spacer module including said bonded mono-SPY-tagged avidin tetramer proteins, to attach a second spacer module, wherein said biotin of said biotinylated second spacer proteins binds to said avidin of said mono-SPY-tagged avidin tetramer proteins;
wherein at least one buffer is present in any one or more of steps a)-d).

43. (canceled)

44. The method of claim 42 comprising e) providing a capture module comprising a SPYtag003 moiety and at least one capturing polypeptide that is adapted to have affinity to biological target molecules, adding said capture module to said second module, wherein said SPYtag003 moiety of said capture module bonds to said SPYcatcher003 moiety of said biotinylated second spacer proteins.

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. A combination product including the magnetic particles that capture biological target molecules for cryo-electron microscope imaging of claim 1 comprising:
a) paramagnetic beads conjugated with streptavidin;
b) optional biotin-3HB-SPYcatcher003 proteins;
c) biotinylated proteins including SAH and SPYcatcher003 moieties,
d) mono-SPY-tagged avidin tetramer proteins; and
e) a capture module comprising a SPYtag003moiety and at least one capturing polypeptide that is adapted to have affinity to biological target molecules;
wherein said 3HB is a three helix bundle and said SAH is a single alpha helix; and
wherein at least two of components a), b), c), d) and e) are provided in separate packaging; and
comprising at least one buffer containing one or more of components a)-e).

59. (canceled)

60. (canceled)

61. (canceled)

62. (canceled)

63. (canceled)

64. (canceled)

65. (canceled)

66. (canceled)

67. (canceled)

68. (canceled)

69. (canceled)

70. (canceled)

71. (canceled)

72. (canceled)

73. (canceled)


SEQUENCE LISTING (23 sequences)

SEQ ID NO: 1 - DNA, 5362 bp, synthetic construct
  misc_feature 1..5362: note = Description of sequence: Plasmid pQE80-His14-bdSUMO-Cys-3HB-SPYcatcher003
  source 1..5362: mol_type = other DNA
ttaagggattttggtcatgacattaacctataaaaataggcgtatcacgaggccctttcgtcttcacctcgagaaatcataaaaaatttatttgctttgtgagcggataacaattataatagattcaattgtgagcggataacaatttcacacagaattcattaaagaggagaaattaaccatgagcaagcatcaccatcattcaggccatcaccataccggacaccaccatcattcaggcagtcatcaccataccggcgagaacctgtattttcagggttcagctgcaggcggtgaagaggataaaaagccagccggaggggaaggtggtggtgctcatattaacttaaaggttaaaggacaggatggcaacgaggtgttcttccgcattaagcgcagtacgcaactgaaaaaactgatgaacgcctactgtgaccgtcaatcagttgacatgactgccattgcgttcttatttgacggtcgccgcctgcgtgcagaacagacaccagacgagttagaaatggaagatggagatgagatcgatgcgatgttgcatcagacaggcggtgcgggtacctgcggtaccctgtataaacaaatggtgcaggagctggaaaaagcccgtgaccgcatggagaagttatacaaagaaatggtggagttaatccagaaagcgatcgaactgatgcgtaaaattttccaggaagtaaaacaggaggttgagaaggccattgaggagatgaaaaaattgtacgatgaagctaaaaaaaagattgagcaaatgattcaacaaattaagcagggaggcgataaacagaaaatggaagagttacttaagcgtgccaaagaagagatgaaaaaagtcaaagacaagatggaaaaattattagaaaagttaaagcaaatcatgcaggaagccaaacaaaagatggagaagctgttaaaacaattaaaggaagagatgaaaaaaatgaaagaaaaaatggaaaagttattgaaggaaatgaagcagcgcatggaggaagtcaaaaagaagatggacggggatgatgaattgttagaaaagattaaaaaaaatatcgatgatttaaagaaaattgccgaggatttaatcaaaaaagccgaggaaaacatcaaggaggccaaaaagatcgcagagcagctggttaagcgcgcaaaacaattaatcgaaaaagcaaagcaagtagcagaggagcttattaagaagatcttgcaattgattgaaaaagcaaaggaaattgctgaaaaggttctgaaaggcggcagtgccatggtaaccaccttatcaggtttatcaggtgagcaaggtccgtccggtgatatgacaactgaagaagatagtgctacccatattaaattctcaaaacgtgatgaggacggccgtgagttagctggtgcaactatggagttgcgtgattcatctggtaaaactattagtacatggatttcagatggacatgtgaaggatttctacctgtatccaggaaaatatacatttgtcgaaaccgcagcaccagacggttatgaggtagcaactccaattgaatttacagttaatgaggacggtcaggttactgtagatggtgaagcaactgaaggtgacgctcatactggatccagtggtagctagtaagcttaattagctgagcttggactcctgttgatagatccagtaatgacctcagaactccatctggatttgttcagaacgctcggttgccgccgggcgttttttattggtgagaatccaagctagccatgaaaataaactgtctgcttacataaacagtaatacaaggggtgttatgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcttggttactcacgactgcgatccccggcaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcacaaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagaattaattcatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatttttttaaggcagttattggtgcccttaaacgcctggggtaatgactctctagcttgaggcatcaaataaaacgaaaggctcagtcgaaagactgggcctttcgttttatctgttgtttgtcggtgaacgctctcctgagtaggacaaatccgccctctagattacgtgcagtcgatgataagctgtcaaacatgagaattgtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcaccattcgatggtgtcggaatttcgggcagcgttgggtcctggccacgggtgcgcatgatctagagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacg

SEQ ID NO: 2 - protein, 360 aa, synthetic construct
  REGION 1..360: note = Description of sequence: Biotin-3HB-SPYcatcher003
  MOD_RES 4: note = biotinylated cysteine
AGTCGTLYKQMVQELEKARDRMEKLYKEMVELIQKAIELMRKIFQEVKQEVEKAIEEMKKLYDEAKKKIEQMIQQIKQGGDKQKMEELLKRAKEEMKKVKDKMEKLLEKLKQIMQEAKQKMEKLLKQLKEEMKKMKEKMEKLLKEMKQRMEEVKKKMDGDDELLEKIKKNIDDLKKIAEDLIKKAEENIKEAKKIAEQLVKRAKQLIEKAKQVAEELIKKILQLIEKAKEIAEKVLKGGSAMVTTLSGLSGEQGPSGDMTTEEDSATHIKFSKRDEDGRELAGATMELRDSSGKTISTWISDGHVKDFYLYPGKYTFVETAAPDGYEVATPIEFTVNEDGQVTVDGEATEGDAHTGSSGS

SEQ ID NO: 3 - DNA, 5314 bp, synthetic construct
  misc_feature 1..5314: note = Description of sequence: Plasmid: pQE80-His14-bdSUMO-Cys-30nm-SAH-SPYcatcher003
  source 1..5314: mol_type = other DNA
ttaagggattttggtcatgacattaacctataaaaataggcgtatcacgaggccctttcgtcttcacctcgagaaatcataaaaaatttatttgctttgtgagcggataacaattataatagattcaattgtgagcggataacaatttcacacagaattcattaaagaggagaaattaaccatgagcaagcatcaccatcattcaggccatcaccataccggacaccaccatcattcaggcagtcatcaccataccggcgagaacctgtattttcagggttcagctgcaggcggtgaagaggataaaaagccagccggaggggaaggtggtggtgctcatattaacttaaaggttaaaggacaggatggcaacgaggtgttcttccgcattaagcgcagtacgcaactgaaaaaactgatgaacgcctactgtgaccgtcaatcagttgacatgactgccattgcgttcttatttgacggtcgccgcctgcgtgcagaacagacaccagacgagttagaaatggaagatggagatgagatcgatgcgatgttgcatcagacaggcggtgcgggtacctgcggtacccagggcggaagcggagaagaggaagagaagaagaaagaagaggaagaaaagaaacaaaaagaagaacaagaaagacttgcaaaagaagaggcagagagaaaacaaaaagaagaacaagaaagacttgcaaaagaagaggcagagagaaaacaaaaggaggaagaagagagaaaacaaaaggaagaagaagagagaaaacaaaaggaggaagaagaaagaaaattaaaggaggaacaagaaagaaaagctgcagaagaaaagaaagctaaagaagaagctgagagaaaggctaaagaagaacaagaaaggaaagctgaagaagagagaaagaagaaagaagaggaagaaagacttgaaagagaaagaaaagagagagaagaacaagaaaagaaagccaaagaagaggcagagagaattgcaaagttagaggctgaaaagaaggcagaagaagaaagaaaagccaaagaagaagaagagagaaaagccaaagaagaagaggaaagaaagaagaaagaggagcaagaaagacttgcaaaagaaaaggaagaagcagaaagaaaagctgcagaggaaaagaaagctaaagaagaacaagaaagaaaagaaaaggaagaagcagaaagaaaacaaagaggctctggcggctctggcggcgccatggtaaccaccttatcaggtttatcaggtgagcaaggtccgtccggtgatatgacaactgaagaagatagtgctacccatattaaattctcaaaacgtgatgaggacggccgtgagttagctggtgcaactatggagttgcgtgattcatctggtaaaactattagtacatggatttcagatggacatgtgaaggatttctacctgtatccaggaaaatatacatttgtcgaaaccgcagcaccagacggttatgaggtagcaactccaattgaatttacagttaatgaggacggtcaggttactgtagatggtgaagcaactgaaggtgacgctcatactggatccagtggtagctagtaagcttaattagctgagcttggactcctgttgatagatccagtaatgacctcagaactccatctggatttgttcagaacgctcggttgccgccgggcgttttttattggtgagaatccaagctagccatgaaaataaactgtctgcttacataaacagtaatacaaggggtgttatgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcttggttactcacgactgcgatccccggcaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcacaaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagaattaattcatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatttttttaaggcagttattggtgcccttaaacgcctggggtaatgactctctagcttgaggcatcaaataaaacgaaaggctcagtcgaaagactgggcctttcgttttatctgttgtttgtcggtgaacgctctcctgagtaggacaaatccgccctctagattacgtgcagtcgatgataagctgtcaaacatgagaattgtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcaccattcgatggtgtcggaatttcgggcagcgttgggtcctggccacgggtgcgcatgatctagagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacg

SEQ ID NO: 4 - protein, 344 aa, synthetic construct
  REGION 1..344: note = Description of sequence: Biotin-30nm-SAH-SPYcatcher003
  MOD_RES 4: note = biotinylated cysteine
AGTCGTQGGSGEEEEKKKEEEEKKQKEEQERLAKEEAERKQKEEQERLAKEEAERKQKEEEERKQKEEEERKQKEEEERKLKEEQERKAAEEKKAKEEAERKAKEEQERKAEEERKKKEEEERLERERKEREEQEKKAKEEAERIAKLEAEKKAEEERKAKEEEERKAKEEEERKKKEEQERLAKEKEEAERKAAEEKKAKEEQERKEKEEAERKQRGSGGSGGAMVTTLSGLSGEQGPSGDMTTEEDSATHIKFSKRDEDGRELAGATMELRDSSGKTISTWISDGHVKDFYLYPGKYTFVETAAPDGYEVATPIEFTVNEDGQVTVDGEATEGDAHTGSSGS

SEQ ID NO: 5 - DNA, 5932 bp, synthetic construct
  misc_feature 1..5932: note = Description of sequence: Plasmid: pQE80-His14-bdSUMO-Cys-60nm-SAH-SPYcatcher003
  source 1..5932: mol_type = other DNA
ttaagggattttggtcatgacattaacctataaaaataggcgtatcacgaggccctttcgtcttcacctcgagaaatcataaaaaatttatttgctttgtgagcggataacaattataatagattcaattgtgagcggataacaatttcacacagaattcattaaagaggagaaattaaccatgagcaagcatcaccatcattcaggccatcaccataccggacaccaccatcattcaggcagtcatcaccataccggcgagaacctgtattttcagggttcagctgcaggcggtgaagaggataaaaagccagccggagggggaggtggtggtgctcatattaacttaaaggttaaaggacaggatggcaacgaggtgttcttccgcattaagcgcagtacgcaactgaaaaaactgatgaacgcctactgtgaccgtcaatcagttgacatgactgccattgcgttcttatttgacggtcgccgcctgcgtgcagaacagacaccagacgagttagaaatggaagatggagatgagatcgatgcgatgttgcatcagacaggcggtgcgggtacctgcggtacccagggcggaagcggagaagaggaagagaagaagaaagaagaggaagaaaagaaacaaaaagaagaacaagaaagacttgcaaaagaagaggcagagagaaaacaaaaagaagaacaagaaagacttgcaaaagaagaggcagagagaaaacaaaaggaggaagaagagagaaaacaaaaggaagaagaagagagaaaacaaaaggaggaagaagaaagaaaattaaaggaggaacaagaaagaaaagctgcagaagaaaagaaagctaaagaagaagctgagagaaaggctaaagaagaacaagaaaggaaagctgaagaagagagaaagaagaaagaagaggaagaaagacttgaaagagaaagaaaagagagagaagaacaagaaaagaaagccaaagaagaggcagagagaattgcaaagttagaggctgaaaagaaggcagaagaagaaagaaaagccaaagaagaagaagagagaaaagccaaagaagaagaggaaagaaagaagaaagaggagcaagaaagacttgcaaaagaaaaggaagaagcagaaagaaaagctgcagaggaaaagaaagctaaagaagaacaagaaagaaaagaaaaggaagaagcagaaagaaaacttaaggaagaggaagagaagaagaaagaagaggaagaaaagaaacaaaaagaagaacaagaaagacttgcaaaagaagaggcagagagaaaacaaaaagaagaacaagaaagacttgcaaaagaagaggcagagagaaaacaaaaggaggaagaagagagaaaacaaaaggaagaagaagagagaaaacaaaaggaggaagaagaaagaaaattaaaggaggaacaagaaagaaaagctgcagaagaaaagaaagctaaagaagaagctgagagaaaggctaaagaagaacaagaaaggaaagctgaagaagagagaaagaagaaagaagaggaagaaagacttgaaagagaaagaaaagagagagaagaacaagaaaagaaagccaaagaagaggcagagagaattgcaaagttagaggctgaaaagaaggcagaagaagaaagaaaagccaaagaagaagaagagagaaaagccaaagaagaagaggaaagaaagaagaaagaggagcaagaaagacttgcaaaagaaaaggaagaagcagaaagaaaagctgcagaggaaaagaaagctaaagaagaacaagaaagaaaagaaaaggaagaagcagaaagaaaacaaagaggctctggcggctctggcggcgccatggtaaccaccttatcaggtttatcaggtgagcaaggtccgtccggtgatatgacaactgaagaagatagtgctacccatattaaattctcaaaacgtgatgaggacggccgtgagttagctggtgcaactatggagttgcgtgattcatctggtaaaactattagtacatggatttcagatggacatgtgaaggatttctacctgtatccaggaaaatatacatttgtcgaaaccgcagcaccagacggttatgaggtagcaactccaattgaatttacagttaatgaggacggtcaggttactgtagatggtgaagcaactgaaggtgacgctcatactggatccagtggtagctagtaagcttaattagctgagcttggactcctgttgatagatccagtaatgacctcagaactccatctggatttgttcagaacgctcggttgccgccgggcgttttttattggtgagaatccaagctagccatgaaaataaactgtctgcttacataaacagtaatacaaggggtgttatgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcttggttactcacgactgcgatccccggcaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcacaaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagaattaattcatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatttttttaaggcagttattggtgcccttaaacgcctggggtaatgactctctagcttgaggcatcaaataaaacgaaaggctcagtcgaaagactgggcctttcgttttatctgttgtttgtcggtgaacgctctcctgagtaggacaaatccgccctctagattacgtgcagtcgatgataagctgtcaaacatgagaattgtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcaccattcgatggtgtcggaatttcgggcagcgttgggtcctggccacgggtgcgcatgatctagagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacg

SEQ ID NO: 6 - protein, 550 aa, synthetic construct
  REGION 1..550: note = Description of sequence: Biotin-60nm-SAH-SPYcatcher003
  MOD_RES 4: note = biotinylated cysteine
AGTCGTQGGSGEEEEKKKEEEEKKQKEEQERLAKEEAERKQKEEQERLAKEEAERKQKEEEERKQKEEEERKQKEEEERKLKEEQERKAAEEKKAKEEAERKAKEEQERKAEEERKKKEEEERLERERKEREEQEKKAKEEAERIAKLEAEKKAEEERKAKEEEERKAKEEEERKKKEEQERLAKEKEEAERKAAEEKKAKEEQERKEKEEAERKLKEEEEKKKEEEEKKQKEEQERLAKEEAERKQKEEQERLAKEEAERKQKEEEERKQKEEEERKQKEEEERKLKEEQERKAAEEKKAKEEAERKAKEEQERKAEEERKKKEEEERLERERKEREEQEKKAKEEAERIAKLEAEKKAEEERKAKEEEERKAKEEEERKKKEEQERLAKEKEEAERKAAEEKKAKEEQERKEKEEAERKQRGSGGSGGAMVTTLSGLSGEQGPSGDMTTEEDSATHIKFSKRDEDGRELAGATMELRDSSGKTISTWISDGHVKDFYLYPGKYTFVETAAPDGYEVATPIEFTVNEDGQVTVDGEATEGDAHTGSSGS

SEQ ID NO: 7 - DNA, 5849 bp, synthetic construct
  misc_feature 1..5849: note = Description of sequence: Plasmid: pET21-SPY-His6-tag streptavidin
  source 1..5849: mol_type = other DNA
ttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatatacatatgcgtggtgttccgcacatcgtaatggttgacgcgtacaaacgttacaaacatcaccatcaccaccatggtggtggcggttctgctgaagctggtatcaccggcacctggtacaaccagctgggatccaccttcatcgttaccgctggtgctgacggtgctctgaccggtacctacgaatccgctgttggtaacgctgaatctagatacgttctgaccggtcgttacgactccgctccggctaccgacggttccggaaccgctctgggttggaccgttgcttggaaaaacaactaccgtaacgctcactccgctaccacctggtctggccagtacgttggtggtgctgaagctcgtatcaacacccagtggttgttgacctccggcaccaccgaagccaacgcgtggaaatccaccctggttggtcacgacaccttcaccaaagttaaaccgtccgctgcttcctaataaaagcttgcggccgcactcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcaatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgcc

SEQ ID NO: 8 - DNA, 5768 bp, synthetic construct
  misc_feature 1..5768: note = Description of sequence: Plasmid: pET21-streptavidin
  source 1..5768: mol_type = other DNA
ttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatatacatatggctgaagctggtatcaccggcacctggtacaaccagctgggatccaccttcatcgttaccgctggtgctgacggtgctctgaccggtacctacgaatccgctgttggtaacgctgaatctagatacgttctgaccggtcgttacgactccgctccggctaccgacggttccggaaccgctctgggttggaccgttgcttggaaaaacaactaccgtaacgctcactccgctaccacctggtctggccagtacgttggtggtgctgaagctcgtatcaacacccagtggttgttgacctccggcaccaccgaagccaacgcgtggaaatccaccctggttggtcacgacaccttcaccaaagttaaaccgtccgctgcttcctaataaaagcttgcggccgcactcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcaatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgcc

SEQ ID NO: 9 - protein, 155 aa, synthetic construct
  REGION 1..155: note = Description of sequence: SPY-His6-tag streptavidin
MRGVPHIVMVDAYKRYKHHHHHHGGGGSAEAGITGTWYNQLGSTFIVTAGADGALTGTYESAVGNAESRYVLTGRYDSAPATDGSGTALGWTVAWKNNYRNAHSATTWSGQYVGGAEARINTQWLLTSGTTEANAWKSTLVGHDTFTKVKPSAAS

SEQ ID NO: 10 - protein, 128 aa, Streptomyces avidinii
  REGION 1..128: note = Description of sequence: Streptavidin
MAEAGITGTWYNQLGSTFIVTAGADGALTGTYESAVGNAESRYVLTGRYDSAPATDGSGTALGWTVAWKNNYRNAHSATTWSGQYVGGAEARINTQWLLTSGTTEANAWKSTLVGHDTFTKVKPSAAS

SEQ ID NO: 11 - DNA, 6157 bp, synthetic construct
  misc_feature 1..6157: note = Description of sequence: Plasmid pET28b-His-SPY-Tandem GFP nanobody
  source 1..6157: mol_type = other DNA
taatacgactcactataggggaattgtgagcggataacaattcccctctagaaataattttgtttaactttaagaaggagatataccatgggcagcagccatcatcatcatcatcatcatcacagcagcggccgtggtgtcccgcacatcgttatggttgacgcgtacaaacgttacaaacatatggcccaagttcagctggttgagagtggtggtgcgctggttcagccgggtggtagtctgcgtctcagttgcgccgcgagcggtttcccggtgaatcgctacagtatgcgctggtaccgtcaagccccgggcaaagaacgcgaatgggttgccggtatgagtagtgccggtgatcgcagcagctatgaagacagcgtgaaaggccgctttaccatcagccgcgatgatgcgcgcaataccgtgtacctccagatgaacagtctgaagccagaggataccgccgtgtactactgcaacgtgaacgtgggcttcgaatattggggtcaaggcacgcaagttaccgttagtggcggcggcggtagcggcggtggcggtagtggtggcggcggcagcggtggcggtggtagcgcccaagttcagctggttgaaagcggcggtcgtctggttcaagccggtgatagtctgcgcctcagttgtgcggccagtggtcgcaccttcagcaccagcgccatggcgtggtttcgtcaagccccgggccgcgaacgcgaatttgttgccgccatcacgtggacggttggcaacaccatcctcggcgatagcgttaaaggccgcttcaccattagccgcgatcgcgcgaaaaacaccgtggatctccagatggacaacctcgaaccggaagataccgcggtgtactattgcagcgcccgtagccgcggctatgtgctgagcgtgctgcgcagcgttgatagctacgactactggggccaaggcacccaagttaccgtgagctaaggatccgaattcgagctccgtcgacaagcttgcggccgcactcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcaatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaat

SEQ ID NO: 12 - protein, 295 aa, synthetic construct
  REGION 1..295: note = Description of sequence: His6-SPYtag-tandem GFP nanobody
MGSSHHHHHHHHSSGRGVPHIVMVDAYKRYKHMAQVQLVESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVSGGGGSGGGGSGGGGSGGGGSAQVQLVESGGRLVQAGDSLRLSCAASGRTFSTSAMAWFRQAPGREREFVAAITWTVGNTILGDSVKGRFTISRDRAKNTVDLQMDNLEPEDTAVYYCSARSRGYVLSVLRSVDSYDYWGQGTQVTVS

SEQ ID NO: 13 - DNA, 5857 bp, synthetic construct
  misc_feature 1..5857: note = Description of sequence: Plasmid: pQE80-His14-bdSUMO-GFP-H1.8
  source 1..5857: mol_type = other DNA
ttaagggattttggtcatgacattaacctataaaaataggcgtatcacgaggccctttcgtcttcacctcgagaaatcataaaaaatttatttgctttgtgagcggataacaattataatagattcaattgtgagcggataacaatttcacacagaattcattaaagaggagaaattaaccatgaatcacaaagtgagcaagcatcaccatcattcaggccatcaccataccggacaccaccatcattcaggcagtcatcaccataccggcgagaacctgtattttcagggttcagctgcaggcggtgaagaggataaaaagccagccggaggggaaggtggtggtgctcatattaacttaaaggttaaaggacaggatggcaacgaggtgttcttccgcattaagcgcagtacgcaactgaaaaaactgatgaacgcctactgtgaccgtcaatcagttgacatgactgccattgcgttcttatttgacggtcgccgcctgcgtgcagaacagacaccagacgagttagaaatggaagatggagatgagatcgatgcgatgttgcatcagacaggcggtatggctcctaagaaggcagttgctgcacctgagggaggcaacaaggaaaatgcagcagtaaaaggatccagtaaagttaaggttaaaagaaaatctatcaaactagtcaagacccaatcacatcccccaaccctgtcgatggtggtggaggtcctgaaaaagaacacggagcggaaagggacctctgtgcaggccattcggacccggattctgtctgcacatcccacagtggatccactgaggctgaagtttttgctacggacggccctgaacaaagggctagagaaggggattctgatcagacctctaaactctagtgcaacaggagctacaggaagattcaaacttgccaaaccagtaaaaactacaaaggctgggaaagaaaatgtagcgtctgaaaacgtagacccaaatgcagagcaggaaacccaaaagaaggccccaaagaaagaaaagaaagcgaagactgagaaagaacccaaaggtgagaaaaccaaagctgtagctaaaaaggccaaggaagattctgatgaaaaacccaaagttgccaaatctaagaaagataaagaggcaaaagaagttgacaaggctaataaagaggcaaaagaagttgacaaggctaataaagaggcaaaagaagttgacaaggctccggcaaagaaaccaaaagccaaaacagaggctgcgaaagctgaggggggtggcaaggcaaagaaggagcccccaaaggccaaagccaaggacgtgaaagcacagaaggactctacagatgaaggtgctccagttaaggctggcaagaaaggaaagaaagtgacaaacggtggtggcggttctggcggcggtggcagcatgagcaaaggggaagaactgtttaccggcgtggtgccgattctggtggagctggatggtgatgtgaatgggcataagtttagcgtgcgtggcgaaggcgagggcgacgcgaccaatggcaagctgaccctgaagttcatttgcaccaccggcaaactgcccgtgccgtggccgaccctggtgaccaccctgacctatggcgtgcagtgctttagccgttatccagatcacatgaaacgtcatgatttctttaagagcgcgatgccggaaggctatgtgcaggaacgtaccattagctttaaggatgatggcacctataaaacccgtgcggaagtgaaatttgagggggacacccttgtaaatcgtattgaactgaaaggcattgacttcaaggaagacggcaatattctgggccataaactggaatacaactttaatagccataacgtgtatattaccgcggataaacagaaaaatggcattaaagcgaatttcaaaatccgtcataatgtggaagatggcagcgttcagctggcggatcactatcagcagaataccccgattggcgatggcccggtgttgctgccggataatcattacctgagcacccagagcgtgctgagcaaggatccgaatgaaaaacgtgatcatatggtgctgttggaatttgtgaccgcagcggggattacccatggcatggatgaactgtataaatagtaagcttaattagctgagcttggactcctgttgatagatccagtaatgacctcagaactccatctggatttgttcagaacgctcggttgccgccgggcgttttttattggtgagaatccaagctagccatgaaaataaactgtctgcttacataaacagtaatacaaggggtgttatgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcttggttactcacgactgcgatccccggcaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcacaaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagaattaattcatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatttttttaaggcagttattggtgcccttaaacgcctggggtaatgactctctagcttgaggcatcaaataaaacgaaaggctcagtcgaaagactgggcctttcgttttatctgttgtttgtcggtgaacgctctcctgagtaggacaaatccgccctctagattacgtgcagtcgatgataagctgtcaaacatgagaattgtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcaccattcgatggtgtcggaatttcgggcagcgttgggtcctggccacgggtgcgcatgatctagagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacg

SEQ ID NO: 14 - protein, 521 aa, synthetic construct
  REGION 1..521: note = Description of sequence: GFP-H1.8
MAPKKAVAAPEGGNKENAAVKGSSKVKVKRKSIKLVKTQSHPPTLSMVVEVLKKNTERKGTSVQAIRTRILSAHPTVDPLRLKFLLRTALNKGLEKGILIRPLNSSATGATGRFKLAKPVKTTKAGKENVASENVDPNAEQETQKKAPKKEKKAKTEKEPKGEKTKAVAKKAKEDSDEKPKVAKSKKDKEAKEVDKANKEAKEVDKANKEAKEVDKAPAKKPKAKTEAAKAEGGGKAKKEPPKAKAKDVKAQKDSTDEGAPVKAGKKGKKVTNGGGGSGGGGSMSKGEELFTGVVPILVELDGDVNGHKFSVRGEGEGDATNGKLTLKFICTTGKLPVPWPTLVTTLTYGVQCFSRYPDHMKRHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSVLSKDPNEKRDHMVLLEFVTAAGITHGMDELYK

SEQ ID NO: 15 - protein, 129 aa, Xenopus laevis
  REGION 1..129: note = Description of sequence: X. laevis H2A peptide
SGRGKQGGKTRAKAKTRSSRAGLQFPVGRVHRLLRKGNYAERVGAGAPVYLAAVLEYLTAEILELAGNAARDNKKTRIIPRHLQLAVRNDEELNKLLGRVTIAQGGVLPNIQSVLLPKKTESSKSAKSK

SEQ ID NO: 16 - protein, 126 aa, Xenopus laevis
  REGION 1..126: note = Description of sequence: X. laevis H2B peptide
MPEPAKSAPAPKKGSKKAVTKTQKKDGKKRRKTRKESYAIYVYKVLKQVHPDTGISSKAMSIMNSFVNDVFERIAGEASRLAHYNKRSTITSREIQTAVRLLLPGELAKHAVSEGTKAVTKYTSAK

SEQ ID NO: 17 - protein, 135 aa, Xenopus laevis
  REGION 1..135: note = Description of sequence: X. laevis H3.2 peptide
ARTKQTARKSTGGKAPRKQLATKAARKSAPATGGVKKPHRYRPGTVALREIRRYQKSTELLIRKLPFQRLVREIAQDFKTDLRFQSSAVMALQEASEAYLVALFEDTNLCAIHAKRVTIMPKDIQLARRIRGERA

SEQ ID NO: 18 - protein, 102 aa, Xenopus laevis
  REGION 1..102: note = Description of sequence: X. laevis H4 peptide
SGRGKGGKGLGKGGAKRHRKVLRDNIQGITKPAIRRLARRGGVKRISGLIYEETRGVLKVFLENVIRDAVTYTEHAKRKTVTAMDVVYALKRQGRTLYGFGG

SEQ ID NO: 19 - DNA, 193 bp, synthetic construct
  misc_feature 1..193: note = Description of sequence: 193 bp 601 DNA
  source 1..193: mol_type = other DNA
atcggaccctatcgcgagccaggcctgagaatccggtgccgaggccgctcaattggtcgtagacagctctagcaccgcttaaacgcacgtacgcgctgtcccccgcgttttaaccgccaaggggattactccctagtctccaggcacgtgtcagatatatacatccaggccttgtgtcgcgaaattcatagat

SEQ ID NO: 20 - DNA, 37 bp, synthetic construct
  misc_feature 1..37: note = Description of sequence: Primer: PCR primer (top) for 193 bp 601 DNA
  source 1..37: mol_type = other DNA
atcggaccctatcgcgagccaggcctgagaatccggt

SEQ ID NO: 21 - DNA, 46 bp, synthetic construct
  misc_feature 1..46: note = Description of sequence: Primer: PCR primer (bottom) for 193 bp 601 DNA
  source 1..46: mol_type = other DNA
atctatgaatttcgcgacacaaggcctggatgtatatatctgacac

SEQ ID NO: 22 - DNA, 4696 bp, synthetic construct
  misc_feature 1..4696: note = Description of sequence: Plasmid: pSPY-GFP nanobody
  source 1..4696: mol_type = other DNA
ttaagggattttggtcatgacattaacctataaaaataggcgtatcacgaggccctttcgtcttcacctcgagaaatcataaaaaatttatttgctttgtgagcggataacaattataatagattcaattgtgagcggataacaatttcacacagaattcattaaagaggagaaattaaccatgaatcacaaagtgagcaagcatcaccatcattcaggccatcaccataccggacaccaccatcattcaggcagtcatcaccataccggcgagaacctgtattttcagggttcagctgcaggcggtgaagaggataaaaagccagccggaggggaaggtggtggtgctcatattaacttaaaggttaaaggacaggatggcaacgaggtgttcttccgcattaagcgcagtacgcaactgaaaaaactgatgaacgcctactgtgaccgtcaatcagttgacatgactgccattgcgttcttatttgacggtcgccgcctgcgtgcagaacagacaccagacgagttagaaatggaagatggagatgagatcgatgcgatgttgcatcagacaggcggtgcgcgtggtgtcccgcacatcgttatggttgacgcgtacaaacgttacaaacatatggcccaagttcagctggttgagagtggtggtgcgctggttcagccgggtggtagtctgcgtctcagttgcgccgcgagcggtttcccggtgaatcgctacagtatgcgctggtaccgtcaagccccgggcaaagaacgcgaatgggttgccggtatgagtagtgccggtgatcgcagcagctatgaagacagcgtgaaaggccgctttaccatcagccgcgatgatgcgcgcaataccgtgtacctccagatgaacagtctgaagccagaggataccgccgtgtactactgcaacgtgaacgtgggcttcgaatattggggtcaaggcacgcaagttaccgttagttagtaagcttaattagctgagcttggactcctgttgatagatccagtaatgacctcagaactccatctggatttgttcagaacgctcggttgccgccgggcgttttttattggtgagaatccaagctagccatgaaaataaactgtctgcttacataaacagtaatacaaggggtgttatgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcttggttactcacgactgcgatccccggcaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcacaaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagaattaattcatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatttttttaaggcagttattggtgcccttaaacgcctggggtaatgactctctagcttgaggcatcaaataaaacgaaaggctcagtcgaaagactgggcctttcgttttatctgttgtttgtcggtgaacgctctcctgagtaggacaaatccgccctctagattacgtgcagtcgatgataagctgtcaaacatgagaattgtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcaccattcgatggtgtcggaatttcgggcagcgttgggtcctggccacgggtgcgcatgatctagagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacg

SEQ ID NO: 23 - protein, 134 aa, synthetic construct
  REGION 1..134: note = Description of sequence: His14-bdSUMO-His-SPY-GFP nanobody
ARGVPHIVMVDAYKRYKHMAQVQLVESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVS